(19) United States
(12) Patent Application Publication
(10) Pub. No.: US 2015/0118229 A1
(43) Pub. Date: Apr. 30, 2015

(54) JAK1 SELECTIVE INHIBITOR AND USES THEREOF

(71) Applicant: ABBVIE INC., North Chicago, IL (US)

(72) Inventors: Jeffrey W. Voss, Holden, MA (US); Heidi S. Camp, Winnetka, IL (US); Robert J. Padley, Lake Bluff, IL (US)

(73) Assignee: ABBVIE INC., North Chicago, IL (US)

(21) Appl. No.: 14/523,052

(22) Filed: Oct. 24, 2014

Related U.S. Application Data
(60) Provisional application No. 62/009,398, filed on Jun. 9, 2014.
(60) Provisional application No. 61/895,292, filed on Oct. 24, 2013.

(51) Int. Cl. A61K 31/4985; A61K 45/06

(52) U.S. Cl. CPC A61K 31/4985; A61K 45/06
USPC 424/134.1; 514/250; 514/161; 514/171; 514/20.5; 424/93.4; 514/226.5; 514/150; 424/133.1

(57) ABSTRACT

The invention relates to the use of a JAK1 kinase-selective inhibitor that has minimal inhibitory activity towards Jak2 kinase for treating a disease, such as an inflammatory disease (e.g., moderate to severe Rheumatoid Arthritis) and/or bone loss, either alone or in combination with a DMARD (disease modifying anti-rheumatic drug), such as methotrexate. The invention also provides pharmaceutical composition, dosage formulation, administration route, and dosage schedule thereof.

### REFERENCE TO RELATED APPLICATIONS

This application claims the benefit of the filing date under 35 U.S.C. 119(e) to U.S. provisional application No. 61/895,292, filed on Oct. 24, 2013, and 62/009,398, filed on Jun. 9, 2014. The entire content of both applications has been incorporated herein by reference.

### BACKGROUND OF THE INVENTION

The protein kinases represent a large family of proteins that play a central role in the regulation of a wide variety of cellular processes and maintenance of cellular function. A partial, non-limiting, list of these kinases include: non-receptor tyrosine kinases such as the Janus kinase family (Jak1, Jak2, Jak3 and Tyk2); the fusion kinases, such as BCR-Abl, focal adhesion kinase (FAK), Fes, Lck and Syk; receptor tyrosine kinases such as platelet-derived growth factor receptor kinase (PDGF-R), the receptor kinase for stem cell factor, c-kit, the hepatocyte growth factor receptor, c-Met, and the fibroblast growth factor receptor, FGFR3; and serine/threonine kinases such as b-RAF, mitogen-activated protein kinases (e.g., MKK6) and SAPK2β. Aberrant kinase activity has been observed in many disease states including benign and malignant proliferative disorders as well as diseases resulting from inappropriate activation of the immune and nervous systems. The compound of this invention selectively inhibits the activity of one or more protein kinases over other related kinases, and are thus expected to be useful in the treatment of diseases mediated by the selectively inhibited kinase(s) while avoiding the undesirable side effects associated with the inhibition of the related kinase(s).

In particular, the Jaks comprise 4 known family members: Jak1, 2, 3, and tyrosine kinase 2 (Tyk2). These cytoplasmic tyrosine kinases are associated with membrane cytokine receptors such as common gamma-chain receptors and the glycoprotein 130 (gp130) transmembrane proteins (Murray, J. Immunol. 178(5):2623-2629, 2007). Almost 40 cytokine receptors signal through combinations of these 4 Jaks and their 7 downstream substrates: the signal transduction activators of transcription (STAT) family members (Ghoreschi et al., Immunol Rev. 228(1):273-287, 2009). Cytokine binding to its receptor initiates Jak activation via trans- and auto-phosphorylation. The Jak in turn phosphorylate cytokine receptor residues, creating binding sites for sarcoma homology 2 (SH2) containing proteins, such as the STAT factors and other regulators, which are subsequently activated by Jak phosphorylation. Activated STATs enter the nucleus initiating expression of survival factors, cytokines, chemokines, and molecules that facilitate leukocyte cellular trafficking (Schindler et al., J. Biol Chem. 282(28):20059-20063, 2007). Jak activation also results in cell proliferation via phosphoinositide 3-kinase (PI3K) and protein kinase B-mediated pathways.

Jak3 and Jak1 are components of the common gamma-chain cytokine receptor complexes, and blockade of either inhibits signaling by inflammatory cytokines: interleukin (IL) −2, 4, 7, 9, 15, and 21 (Ghoreschi et al., Immunol. Rev. 228(1):273-287, 2009). By contrast, other pathologically relevant cytokines, such as IL-6, depend uniquely on Jak1. Hence, Jak1 blockade inhibits signaling of many proinflammatory cytokines (Guschin et al., EMBO J. 14(7):1421-1429, 1995). Clinical efficacy in RA has been observed with the IL-6 receptor neutralizing antibody, tocilizumab (Maini et al., Arthritis Rheum. 54(9):2817-2829, 2006).

Humans deficient in Jak1 and Jak2 have not been described. Mice lacking Jak1 die perinatally (Schindler et al., J. Biol Chem. 282(28):20059-20063, 2007). Jak2 deficiency in mice also is lethal, with Jak2^{-/-} embryos dying between Day 12 and Day 13 after conception because of deficits in erythropoiesis (Neubauer et al., Cell 93(3):397-409, 1998). Jak3 deficiency has been described in humans and presents as severe combined immunodeficiency in the first few months of life, with symptoms such as failure to thrive, severe and recurrent infections, thrush, and diarrhea. Infants with Jak3 deficiency have an absence of circulating T cells and NK cells and abnormal B cell function. Tyk2-deficiency additionally has been described in humans, manifesting with impaired antimicrobial responses, elevated serum IgE, and atopic dermatitis (Minegishi et al., Immunity 25(5):745-755, 2006).

Given the high degree of structural similarity between Jak1 and Jak2 (Williams et al., J. Mol. Biol. 387(1):219-232, 2009), the literature suggests that the majority of Jak1 inhibitors also inhibit Jak2 (Incyte Corp. press release, 10 Nov. 2010; Changelian et al., Science 302(5646):875-878, 2003).

Anti-cytokine therapies have become standard in the treatment of rheumatoid arthritis (RA). In humans, a growing body of evidence suggests that Jak1 inhibition is an effective therapy for the treatment of signs and symptoms of RA. Multiple clinical trials administering Pfizer's Jak1/3 inhibitor tofacitinib (Phase 3 trial) (Kremer et al., Arthritis Rheum. 60(7):1895-1905, 2009; Riese et al., Best Pract. Res. Clin. Rheumatol. 24(4):513-526, 2010), Incyte/Lilly's Jak1/2 inhibitor INCB-28050/LY3009104 (Phase 2b) (Incyte Corp. press release, 10 Nov. 2010), or Galapagos' Jak1 inhibitor GLP0634 (Phase 2a) (Galapagos NV press release, 22 Nov. 2011) have demonstrated statistically significant efficacy in this disease.

Tofacitinib, a nonselective inhibitor of Jak1, Jak2, and Jak3, has been approved in the United States and additional countries around the world for the indication of adult patients with moderately to severely active RA who have had an inadequate response or intolerance to methotrexate (MTX), used as monotherapy or in combination with MTX or other nonbiologic DMARDs. Safety data from Phase 2 and Phase 3 studies in patients (Fleischmann, Curr. Opin. Rheumatol. 24(3):335-341, 2012; Kremer et al., Arthritis Rheum. 64(4):970-981, 2012; Fleischmann et al., Arthritis Rheum. 64(3):617-629, 2012) with RA for tofacitinib compared with placebo have indicated that the most common serious adverse reactions are infections, including pneumonia, cellulitis, herpes zoster, and urinary tract infection. In addition, tuberculosis (including cases of disseminated tuberculosis) and opportunistic infections such as other mycobacterial infections, cryptococcus, esophageal candidiasis, pneumocystosis, multidermatomal herpes zoster, cytomegalovirus, and BK virus were reported. Lymphoma and other malignancies have been observed in patients treated with tofacitinib. Epstein-Ban virus-associated post-transplant lymphoproliferative disorder has been observed at an increased rate in renal transplant patients treated with tofacitinib and concomitant immunosuppressive medications. Gastrointestinal perforations in patients receiving tofacitinib also were reported.

In addition, laboratory abnormalities have been described, including dose-related decreases in absolute neutrophil counts as well as hemoglobin. Furthermore, small increases in liver transaminases (alanine aminotransferase [ALT], aspartate aminotransferase [AST]) and serum creatinine, and elevated LDL, HDL, and total cholesterol levels have been reported.

A Phase 2 study of VX-509 (inhibitor of Jak3) in patients with RA also has shown an increased risk of infections and increases in lipid levels (Fleischmann et al., Arthritis Rheum. 63:LB3, 2011).

A 52-week, open-label, long-term extension Phase 2b study of baricitinib—an orally administered selective Jak1 and Jak2 inhibitor—in 201 patients with active RA found no opportunistic infections, cases of tuberculosis, or lymphomas. Clinically significant laboratory abnormalities were infrequently observed (increased ALT, anemia, increased creatine kinase [CK], pancytopenia, reported in 1 subject each); 1 subject discontinued due to a laboratory abnormality (increased ALT). One death occurred and was attributed to presumed myocardial infarction (Keystone et al., Ann. Rheum. Dis. 71(Suppl 3):152, 2012; Genovese et al., Arthritis Rheum. 64(Suppl 10):2487, 2012; Taylor et al., abstract OP0047, EULAR 2013, the Annual Congress of the European League Against Rheumatism. 2013 Jun. 12-15; Madrid, Spain).

Despite the seemingly numerous treatment options, however, many RA patients fail to experience substantial decreases in disease activity. Although earlier studies have shown that Jak blockade may be effective in managing disease and achieving remission, the first generation Jak inhibitors (such as tofacitinib and baricitinib) have failed to reach their full potential, at least partly due to their tolerability and safety issues that limit dose.

Specifically, the first generation Jak inhibitors tofacitinib and baricitinib have been characterized as Jak1/Jak3 and Jak1/Jak2 inhibitors, respectively (Fridman et al., J. Immunol., 184:5298-5307, 2010; Meyer et al., J. Inflamm. (Lond.) 7:41, 2010; and Taylor et al., Rheumatology 52:i44-i55, 2013). Despite the initial encouraging results, these first generation Jak inhibitors have failed to reach their full potential due to tolerability issues that limited dose (Fleischmann et al., Curr. Opin. Rheumatol. 24:335-341, 2012; Riese et al., Best Pract. Res. Clin. Rheumatol. 24:513-526, 2010). JAKs are known to play roles in the regulation of over forty pathways (Murray, J. Immunol 0.178:2623-2629, 2007). However, despite the high selectivity of these two compounds for JAKs over other kinase families, these inhibitors may not be optimally selective for kinases within the JAK family. For instance, incidence of severe anemia was reported to be a dose limiting factor during Tofacitinib Phase II development in RA (Pfizer, Investigators Brochure. In FDA Advisory Board (Bethesda Md.), 2012; Riese et al., Best Pract. Res. Clin. Rheumatol. 24:513-526, 2010). Moreover, increases in herpes virus infections, potentially secondary to decreases in NK cell counts, were reported in Phase III tofacitinib trials (O'Shea et al., Ann. Rheum. Dis. 72(Suppl 2):ii111-115, 2013; Pfizer, Investigators Brochure. In FDA Advisory Board (Bethesda Md.), 2012). It is reasonable that these effects could arise due to inhibition of EPO and IL-15 signaling via Jak2 and Jak3 respectively (Jost and Altfeld, Annu. Rev. Immunol. 31:163-194, 2013; Kennedy et al., J. Exp. Med. 191:771-780, 2000; and Richmond et al., Trends Cell Biol. 15:146-155, 2005). Indeed, failure of interventions to treat anemia associated with RA may limit chances for a fully successful response to treatment.

Thus there is a medical need unmet by the current treatment options using Jak inhibitors.

### SUMMARY OF THE INVENTION

Compound 1 is a second generation Jak inhibitor engineered for increased selectivity for Jak1, using structural predictions that indicated the potential for differential binding interactions outside the ATP-binding active site of Jak1 but not Jak2 and the other related Jaks. The engineering of Compound 1 was based on subtle differences between Jak1 and Jak2 and Jak3 revealed through structural analysis of the enzymes which in turn provided hypotheses to drive structure activity relationships using medicinal chemistry. Compared to tofacitinib, Compound 1 has low potency against Jak3, as well as a modest but significantly improved Jak1/Jak2 window based on in vitro enzymology. Although the biochemical selectivity of Compound 1 translated into modest improvements in cellular selectivity, a surprisingly substantial relative improvements in in vivo pharmacological assessments of Jak-dependent physiology was observed. When dosed in healthy human subjects, Compound 1 has pharmacodynamic (PD) effects similar to those observed in rats, indicating translation of its biochemical profile between species, including reduced effects on NK cells compared to tofacitinib at efficacious exposures. These properties portend that Compound 1 possesses an improved side-effect profile in RA patients compared to existing agents, thus enabling dose escalation and higher levels of efficacy, without triggering significant undesirable side effects that limited the usefulness of the first second generation Jak inhibitors.

Specifically, partly based on several Jak1- and Jak2-dependent cellular assays and in vivo experiments comparing efficacy in an arthritis rat disease model (Rat AIA, [Jak1 inhibition]) with impact on erythropoiesis (EPO-stimulated generation of reticulocytes, [Jak2 inhibition]), Applicants have demonstrated that Compound 1 has minimal impact on Jak2 inhibition at efficacious drug levels that inhibit Jak1, and is therefore a Jak1-selective inhibitor. On the basis of this differentiated selectivity profile for the inhibition of Jaks, especially its preferential/selective inhibition of Jak1 over Jak2 and the other related JAKs (e.g., Jak3 and Tyk2), Compound 1 has the potential for an improved benefit: risk profile compared to that of the other Jak inhibitors currently in clinical trials, as well as other therapeutic strategies for patients with RA and other inflammatory diseases or autoimmune diseases in which Jak1 activity is detrimental.

Thus in one aspect, the invention provides a method of selectively inhibiting a Janus Kinase 1 (Jak1) in a human, comprising administering to the human an effective amount of the free base form of a compound, wherein Jak1 activity is preferentially inhibited over activity of Jak2, activity of Jak3, and activity of Tyk2, and less than 50%, 40%, 30%, 20%, 10%, or 5% of Jak2 and/or Jak3 activity is inhibited in the human, and wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

In a related aspect, the invention provides a method of treating in a human an autoimmune disease or disorder, or an inflammatory disease or disorder, the method comprising administering to the human an effective amount of the free base form of a compound, wherein the effective amount reduces reticulocyte, NK cell, NKT cell, iNKT cell, or CD8⁺ cell count by no more than 50%, 40%, 30%, 25%, 20%, 15%, 10%, 5% relative to a pre-treatment level, and, wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

In yet another related aspect, the invention provides a method of treating in a human an autoimmune disease or disorder, or an inflammatory disease or disorder, the method comprising administering to the human an effective amount of the free base form of a compound, wherein the effective amount produces an AUC₀₋₂₄ of between 0.10-1.1 μg·hr/mL (or between 0.128-1.058 μg·hr/mL) of free base equivalent of the compound, and, wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

In certain embodiments, more than 50%, 60%, 70%, 80%, 90%, 95%, 99% of Jak1 activity is inhibited in the human.

In certain embodiments, the human is in need of treatment for a condition treatable by inhibition of Jak1 activity. For example, the condition may be an inflammatory disease/disorder, or an autoimmune disease/disorder, such as Rheumatoid Arthritis (RA), Crohn's disease, ankylosing spondylitis (AS), psoriatic arthritis, psoriasis, ulcerative colitis, systemic lupus erythematosus (SLE), diabetic nephropathy, dry eye syndrome, Sjogren's Syndrome, alopecia areata, vitiligo, or atopic dermatitis.

Crohn's disease (CD) encompasses a spectrum of clinical and pathological processes manifested by focal asymmetric, transmural, and occasionally granulomatous inflammation that can affect any segment of the gastrointestinal tract. The disease can affect persons of any age, and its onset is most common in the second and third decades. Females are affected slightly more than males, and the risk for disease is higher in some ethnic groups. In North America, the incidence of CD is estimated to be 3.1 to 14.6 cases per 100,000 persons. Prevalence rates range from 26 to 99 cases per 100,000 persons. In Europe, CD has an incidence of 0.7 to 9.8 cases per 100,000 persons and a prevalence of 8.3 to 214 cases per 100,000 persons.

CD has been characterized as a progressive disease that leads to complications. In a population based study from southeastern Norway, a substantial number of patients demonstrated a stricturing or penetrating phenotype at 10 years after diagnosis. Moreover, approximately 80% of patients diagnosed with CD will require at least one surgery related to the disease at some point in time.

Given that no known medical or surgical cure currently exists for CD, the therapeutic strategy is to reduce symptoms, improve quality of life, reduce endoscopic evidence of inflammation, and minimize short- and long-term toxicity and complications. Currently, patients with moderate to severe disease are usually treated with conventional pharmacologic interventions, which include corticosteroids and immunomodulatory agents such as azathioprine, 6-mercaptopurine (6-MP), or methotrexate (MTX). The potential risks from long-term use of corticosteroids are well-known. Adverse events (AEs) associated with short-term use of corticosteroids include acne, moon face, edema, skin striae, glucose intolerance, and sleep/mood disturbances; potential AEs observed with longer term use (usually 12 weeks or longer but sometimes shorter durations) include posterior subcapsular cataracts, osteoporosis, osteonecrosis of the femoral head, myopathy, and susceptibility to infection. The safety risks for AZA and 6-MP include pancreatitis, bone marrow depression, infectious complications, and malignant neoplasms. MTX may be associated with bone marrow depression and liver and pulmonary toxicity. Patients who do not respond to conventional therapies may be treated with biologics, such as anti-TNF α therapies. Potential risks with anti-TNF α include infusion or injection site reactions, serious infections, lymphoma, heart failure, lupus-like syndromes, and demyelinating conditions.

Despite the beneficial results achieved with the available anti-TNF α agents, approximately 40% of patients who receive them for the first time do not have a clinically meaningful response (primary non responders). Among patients who initially respond and continue to receive maintenance treatment for longer durations, approximately 38% become non-responders after 6 months and approximately 50% become non-responders at 1 year (secondary non-responders). Patients who initially respond to a first anti-TNFα agent but then lose response tend to have lower response and remission rates to the second anti-TNFα agent. For those patients who are unable to tolerate anti-TNFα therapies and/or have had an insufficient response to treatment with an anti-TNFα therapy, current options for treatment are limited, and the patients may be subjected to repeated courses of corticosteroids, which are associated with a wide ranging spectrum of toxic effects affecting multiple organ systems.

A new class of biologics, anti-integrin antibodies, has been studied in patients with prior anti-TNFα use. Natalizumab, a humanized monoclonal antibody to α4β1 and α4β7 integrins, showed promise for patients with prior exposure to anti-TNF-α therapy; more than half of the patients had a response to the induction regimen. However, natalizumab's use after approval in 2008 has been severely limited due to the serious risk for progressive multifocal leukoencephalopathy (PML) attributed to activation of the latent JC virus. Another anti-integrin antibody, vedolizumab, a monoclonal antibody that binds to the α4β7 integrin and inhibits the migration of memory T-lymphocytes across the endothelium into inflamed gastrointestinal parenchymal tissue, was recently approved for adult patients with moderately to severely active CD who have had an inadequate response with, lost response to, or were intolerant to a TNF blocker or immunomodulator.

Tofacitinib is a non-selective JAK inhibitor targeting JAK1, JAK2 and JAK3, but most potently inhibits JAK3. Although tofacitinib improves the clinical signs and symptoms of RA, questions remain surrounding the safety profile regarding apparent increases in the incidences of serious infection, malignancies, herpes zoster, and hematologic adverse events. Tofacitinib has also been associated with reduced levels of hemoglobin, absolute lymphocytes counts, and total white blood cell counts in some subjects and also increased serum creatinine; total cholesterol, LDL cholesterol (LDL-C), and HDL cholesterol (HDL-C); and liver transaminases (ALT and AST). The increases in serum creatinine, lipids, and liver transaminase values typically have been asymptomatic, reversible, and were not associated with any overt declines in renal or hepatic function.

Clearly, the medical need for additional therapeutic options in CD for patients with inadequate response to or intolerance to conventional therapies and anti-TNFα agents remains.

Compound 1 is a novel JAK1 selective inhibitor with minimal inhibitory effects on JAK2 and JAK3, which could potentially minimize some of the reported safety concerns with non-selective JAK inhibition which are thought to be mediated by inhibition of JAK2 and JAK3 signaling pathways. The following supportive findings suggest that Compound 1 is effective for treating CD patients: 1) demonstrated improved potency of Compound 1 versus tofacitinib in preclinical models of inflammation; 2) confirmed JAK1 selectivity of Compound 1 in both preclinical and clinical settings; 3) acceptable preclinical toxicological findings in chronic toxicity studies in two species; 4) acceptable safety and tolerability profile of Compound 1 in single ascending dose (SAD) and multiple ascending dose (MAD) studies in healthy volunteers; and, 5) evidence that JAK inhibition in preclinical models of inflammatory bowel disease (IBD) results in clinical and endoscopic improvement.

The Crohn's disease may be moderately to severely active Crohn's disease (CD) in an adult. In certain embodiments, the adult is newly diagnosed of CD (e.g., having colonic or ileocolonic Crohn's disease for ≧3 months), or is inadequately responding to or has discontinued therapy due to loss of response to or intolerance to a first line therapy or an anti-TNFα therapy (e.g., azathioprine, 6-mercaptopurine (6-MP), aminosalicylate (e.g., sulfasalazine, mesalamine), corticosteroid (e.g., prednisone or prednisone equivalent, budesonide), probiotic, methotrexate, cyclosporine, tacrolimus, metronidazole, ciprofloxacin, leflunomide, chloroquine, hydroxychloroquine, penicillamine, tocilzumab, anakinra, abatacept, rituximab, efalizumab, belimumab, tofacitinib, baricitinib, golimumab, vedolizumab, natalizumab, ustekinumab, etanercept, infliximab, adalimumab, certolizumab pegol, or a JAK inhibitor). Representative Jak inhibitors include: ruxolitinib, tofacitinib or CP-690550, baricitinib (LY3009104, INCB28050), CYT387, GLPG0634, GSK2586184, lestaurtinib, pacritinib (SB1518), and TG101348.

In certain embodiments, the adult having the Crohn's disease may have an average daily liquid/very soft stool frequency score of ≧2.5 or average daily abdominal pain score of ≧2.0; and CDAI≧220 and ≦450. In certain embodiments, the adult having the Crohn's disease may have a simplified endoscopic score for Crohn's disease (SES-CD) of ≧6, or ≧4 for subjects with disease limited to the ileum.

The RA may be moderately to severely active RA in an adult. In certain embodiments, RA-associated bone loss or bone erosion in the adult is inhibited. In certain embodiments, the adult is newly diagnosed of RA, is inadequately responding to (oral or biologic) DMARDs, biologics or an anti-TNFα therapy, or has discontinued therapy due to loss of response to or unacceptable toxicity from methotrexate, chloroquine, azathioprine, hydroxychloroquine, penicillamine, sulfasalazine, leflunomide, tocilzumab, anakinra, abatacept, certolizumab pegol, tofacitinib, golimumab, baricitinib, etanercept, infliximab, or adalimumab.

In certain embodiments, the method does not substantially reduce or inhibit common gamma chain signaling, e.g., common gamma chain signaling through Jak1 and Jak3. In certain embodiments, the common gamma chain signaling is stimulated by one or more of: IL-2, IL-4, IL-7, IL-9, IL-15, and IL-21. In certain embodiments, the method reduces or inhibits common gamma chain signaling by no more than 50%, 40%, 30%, 25%, 20%, 15%, 10%, 5% or less, as compared to mock/sham treatment control in a matching sample.

In certain embodiments, the method does not substantially reduce NK cell count, NKT cell count, iNKT cell count, and/or CD8⁺ cell count. In certain embodiments, NK cell count, NKT cell count, iNKT cell count, and/or CD8⁺ cell count are determined using the methods described herein below.

In certain embodiments, the method does not substantially inhibit erythropoiesis, granulocyte/monocyte-colony stimulating factor (GM-CSF) signaling, or emergency myelopoiesis in response to microbial infection in the human.

In certain embodiments, the effective amount reduces reticulocyte, NK cell, NKT cell, iNKT cell, or CD8⁺ cell count by no more than 50%, 40%, 30%, 25%, 20%, 15%, 10%, 5% relative to a pre-treatment level, over a treatment period of at least 14 days (e.g., at least about 29 days or about a month, 3 months, 6 months, 9 months, 1 year, 2 years, 5 years, 10 years, 20 years, 50 years etc.).

In certain embodiments, erythropoiesis is measured by circulating reticulocyte count, or by hemoglobin (Hb) level (g/dL).

In certain embodiments, the human has anemia, or has a whole blood hemoglobin level of less than 12, 11, 10, 9, 8, 7, 6, or 5 g/dL. For example, the whole blood hemoglobin level may be measured by standard clinical laboratory methods, such as CBC (complete blood count) test from a whole blood sample.

In certain embodiments, the compound is administered to the human until a substantially steady level of AUC₀₋₂₄ of between 0.10-1.1 μg·hr/mL of free base equivalent of the compound is reached. For example, the compound may be administered to the human twice daily (BID) in equal amounts. In a specific embodiment, the compound is administered to the human twice daily, each time at a dose of about 3-24 mg (e.g., 3, 6, 9, 12, 18, or 24 mg) of free base equivalent of the compound. In other embodiments, the compound may be administered to the human once daily (QD). In a specific embodiment, the compound is administered to the human once daily, at a dose of about 18 mg or 24 mg of free base equivalent of the compound.

In certain embodiments, the method further comprises maintaining the AUC₀₋₂₄ at substantially the same level over a treatment period, such as a period of at least 14 days, e.g., at least one month, 3 months, 6 months, 9 months, 1 year, 2 years, 5 years, 10 years, 20 years, 50 years etc.

In certain embodiments, inhibition of Jak1 and/or Jak3 activity is determined by measuring ex-vivo stimulated IL-6 dependent STAT3 phosphorylation, ex vivo stimulated IL-7-dependent STAT5 phosphorylation, and/or by determining peripheral NK cell counts.

In certain embodiments, inhibition of Jak2 activity is determined by measuring a common beta chain cytokine (e.g., GM-CSF, IL-3, or IL-5) dependent STAT5 phosphorylation. For example, in certain embodiments, inhibition of Jak2 activity is determined by measuring GM-CSF dependent STAT5 phosphorylation, such as by ex vivo stimulated GM-CSF dependent STAT5 phosphorylation.

In certain embodiments, any of the ex vivo cytokine-stimulated STAT phosphorylation assays is performed using a sample derived from a whole blood sample from an individual/patient.

In other embodiments, inhibition of Jak2 activity is determined by measuring EPO dependent STAT5 phosphorylation.

In certain embodiments, the method further comprises administering to the human one or more additional agents which modulate a mammalian immune system or which are anti-inflammatory agents. The one or more additional agents may be selected from the group consisting of: aspirin, acetaminophen, aminosalicylate, ciprofloxacin, corticosteroid, cyclosporine, metronidazole, probiotic, tacrolimus, ibuprofen, naproxen, piroxicam, prednisolone, dexamethasone, anti-inflammatory steroid, methotrexate, chloroquine, azathioprine, hydroxychloroquine, penicillamine, sulfasalazine, leflunomide, tocilzumab, anakinra, abatacept, certolizumab pegol, golimumab, vedolizumab, natalizumab, ustekinumab, rituximab, efalizumab, belimumab, etanercept, infliximab, adalimumab, or an immune modulator (e.g., activator) for CD4⁺CD25⁺ T_{reg} cells.

In certain embodiments, the method further comprises: (1) identifying a human subject administered with the compound but having inadequate or suboptimal response or therapeutic efficacy; (2) determining reticulocyte, NK cell, NKT cell, iNKT cell, and/or CD8⁺ cell count of the human subject, wherein a decrease in reticulocyte, NK cell, NKT cell, iNKT cell, or CD8⁺ cell count of no more than 30%, 25%, 20%, 15%, or 10% compared to a pre-treatment baseline level of reticulocyte, NK cell, NKT cell, iNKT cell, or CD8⁺ cell count, respectively, is indicative that the human subject is a candidate for dose escalation; (3) administering to the candidate an escalated dose of the compound. In certain embodiments, the method further comprises repeating steps (1)-(3) until a desired outcome is achieved.

In another related aspect, the invention provides a pharmaceutical formulation for treating an autoimmune disease or disorder, or an inflammatory disease or disorder, the pharmaceutical composition comprising: (1) a unit dose of the free base form of the compound (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, and (2) a pharmaceutically acceptable excipient, wherein the unit dose, upon administration to an adult human twice daily (BID), produces an AUC₀₋₂₄ of between 0.10-1.1 μg·hr/mL of free base equivalent of the compound.

In a related aspect, the invention provides a pharmaceutical formulation for treating an autoimmune disease or disorder, or an inflammatory disease or disorder, the pharmaceutical composition comprising: (1) a unit dose of the free base form of the compound (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, and (2) a pharmaceutically acceptable excipient, wherein the unit dose, upon administration to an adult human twice daily (BID), preferentially inhibits activity of Jak1 over activity of Jak2, activity of Jak3, and activity of Tyk2, and inhibits less than 50%, 40%, 30%, 25%, 20%, 15%, 10%, or 5% of Jak2 and/or Jak3 activity in the human.

In another related aspect, the invention provides a pharmaceutical formulation for treating an autoimmune disease or disorder, or an inflammatory disease or disorder, the pharmaceutical composition comprising: (1) a unit dose of the free base form of the compound (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, and (2) a pharmaceutically acceptable excipient, wherein the unit dose, upon administration to an adult human twice daily (BID), reduces reticulocyte, NK cell, NKT cell, iNKT cell, and/or CD8⁺ cell count by no more than 50%, 40%, 30%, 25%, 20%, 15%, 10%, 5% relative to a pre-treatment level.

In certain embodiments, the unit dose is a capsule. In certain embodiments, the unit dose is 0.5, 1, 3, 6, 9, 12, 18, or 24 mg of free base equivalent of the compound.

In certain embodiments, the autoimmune disease or disorder, or inflammatory disease or disorder is Crohn's disease (e.g., moderately to severely active Crohn's disease (CD)) in an adult.

In certain embodiments, the autoimmune disease or disorder, or inflammatory disease or disorder, is Rheumatoid Arthritis (RA), such as moderately to severely active RA in adult.

In certain embodiments, the pharmaceutically acceptable excipient comprises microcrystalline cellulose, dibasic calcium phosphate, magnesium stearate, croscarmellose sodium, hydroxypropyl cellulose, or a mixture thereof.

In certain embodiments, the pharmaceutical formulation is formulated for oral (e.g., selective release in certain parts of the small intestine), topical, dermal, intra-luminal (e.g., via enema for GI or colon indications), or ophthalmic administration.

It should be understood that any embodiment described herein, including embodiment described only under one aspect of the invention, is contemplated to be able to combine with any one or more other embodiments, unless inappropriate or specifically disclaimed.

### BRIEF DESCRIPTION OF THE DRAWINGS

FIG. 1 shows Compound 1 (free base form) (left panel) and Tofacitinib (right panel) IC₅₀ for Jak isoforms Jak1, Jak2, Jak3, and Tyk2, measured in the presence of 0.1 mM ATP. The IC₅₀ values taken from these curves are summarized in the table in the figure. Compound 1 is an ATP competitive inhibitor of Jak1 (data not shown), and displays good selectivity across a panel of 60+ protein kinases (data not shown). It is about 3-fold less potent against Jak2 and about 54-fold less potent against Jak3 recombinant kinase domain proteins. In contrast, tofacitinib is equally potent against Jak1, Jak2, and Jak3, as has been reported elsewhere (Meyer et al., J. Inflamm. (Lond.) 7:41, 2010).

FIG. 2 shows effect of Compound 1 on ConA-induced IFN-γ in Lewis rats.

FIG. 3 shows effect of Compound 1 on adjuvant-induced Arthritis (AIA) in Lewis Rats, as represented by dose and exposure response curves.

FIGS. 4A-4C show effect of Compound 1 on AIA in Lewis Rats—the effect of treatment on bone erosion as represented by MicroCT scanning, showing measured change in tarsal bone volume (FIG. 4A); representative tarsal bone from rats treated with vehicle (FIG. 4B) or Compound 1 (FIG. 4C) at 3 mg/kg bid, respectively.

FIG. 5A shows the exposure response relationship in rat between orally dosed Compound 1 and ex vivo stimulated pSTAT5.

FIG. 5B shows inhibition of cytokine-induced STAT (i.e., STAT3 or STAT5) phosphorylation is dose responsive. Healthy human subjects were dosed orally with Compound 1 at the various indicated doses. IL6 or IL7 was added to blood samples taken from the subjects at the time points indicated (hr), and effects of Compound 1 exposure on STAT3 or STAT5 phosphorylation, respectively, were assessed by flow cytometry.

FIG. 5C shows the result of inhibiting IL-6 or IL-7 stimulated STAT3 and STAT5 phosphorylation, respectively, by Compound 1 and tofacitinib as indicated at 1, 6, and 12 hours post oral dosing of the compounds. It shows that 5 mg of tofacitinib and 3 mg of Compound 1 cause similar level of inhibition of IL6-induced STAT3 phosphorylation (as measured by % inhibition of pSTAT3) when dosed orally in human. The figure also shows that it requires 12 mg of Compound 1 to cause the same level of inhibition of IL-7-induced pSTAT5 as 5 mg of tofacitinib in human.

FIG. 5D shows the result of inhibiting GM-CSF induced STAT5 phosphorylation at 1 hour post the indicated dosages. Inhibition was not observed at lower doses or later time points. The figure shows that 24 mg but not 12 mg of Compound 1 dosed orally in human leads to partial inhibition of Jak2 signaling as assessed by inhibition of GMCSF-induced pSTAT5.

FIGS. 6A and 6B show Compound 1 exposure response relationships in rats for peripheral NK cell counts. FIG. 6A shows % decrease in NK cell counts vs. Compound 1 dose as measured by log AUC₀₋₂₄. FIG. 6B shows a substantially linear relationship between % pSTAT5 decrease at Cₘᵢₙ and % NK cell count decrease at 2 weeks.

FIG. 7 shows decreases in NK cells observed in healthy rats compared to the corresponding effect of exposure on disease activity. NK=natural killer; PS=paw swelling. The grey box highlights the exposure range expected for the doses defined for multiple ascending dose studies in humans.

FIG. 8 shows Compound 1 plasma concentrations (Left) and Area Under the Curve (AUC, Right) following 50, 100 or 200 mg/kg/day oral doses in rats. Results are represented as Mean (±) SD; n=5/gender.

FIG. 9 shows Compound 1 plasma concentrations (Left), Area Under the Curve (AUC) and dose-normalized AUC (AUC/D) following 0.5, 1.5, 3, or 5 mg/kg/day multiple oral dosing in dog, shown as Mean (±) SD.

FIG. 10 shows plasma exposures for predicted efficacious exposure, range of human exposures, and nonclinical NOAEL (No-Observed-Adverse-Effect Level) for Compound 1.

FIG. 11A shows that erythropoietin (EPO) injected i.v. resulted in modest but precise reticulocytosis in rats. In contrast, PBS control had negligible (if any) effect.

FIG. 11B shows that the effects of Compound 1 on reticulocyte deployment are less than that of Tofacitinib over the exposure range efficacious in the rat AIA disease model.

FIG. 11C shows that reticulocyte deployment at efficacious exposures is closely related to Jak1/Jak2 selectivity. Compound 7, a highly selective Jak1 inhibitor related to Compound 1, and Baricitinib (Gras, Drugs of the Future 38:611-617, 2013), an inhibitor of Jak1 and Jak2, were also included. There was good alignment between the level of Jak1/Jak2 cellular selectivity and compound effects on reticulocyte deployment. The trend established by these compounds supports the notion that increased Jak1/Jak2 selectivity correlates with smaller effects on EPO signaling per unit efficacy.

FIG. 12 is composite exposure/response curves of disease model efficacy and peripheral NK cell counts. It shows that the effects of Compound 1 on peripheral NK cell counts are less than that of Tofacitinib over the efficacious exposure range. The log concentrations for Tofacitinib and Compound 1 are expressed as AUC exposure (ng·hr/mL). Each NK cell data point represents the average of four rats in individual dose groups. Each paw swelling data point represents the average of nine rats.

FIG. 13 is a plot of NK cell effects per unit efficacy. It suggests that high doses of Compound 1 should have relatively minimum effects on NK cells counts compared to Tofacitinib in view of experimental data from rat.

FIG. 14A shows change in peripheral NK cells in response to 3 mg, 6 mg, 12 mg and 24 mg Compound 1 dosed twice a day (bid) in healthy human subjects for 14 days.

FIG. 14B shows change in peripheral NKT cells in response to 3 mg, 6 mg, 12 mg and 24 mg Compound 1 dosed twice a day in healthy human subjects for 14 days.

FIG. 14C shows change in circulating reticulocytes in response to 3 mg, 6 mg, 12 mg and 24 mg Compound 1 dosed twice a day in healthy human subjects for 14 days.

FIG. 15A shows change in circulating reticulocytes in response to 6 mg, 12 mg, and 24 mg Compound 1 dosed twice a day for 26 days and a single dose on the 27^{th} day in RA patients/subjects.

FIG. 15B shows change in hemoglobin (Hb) levels in response to 6 mg, 12 mg, and 24 mg Compound 1 dosed twice a day for 26 days and a single dose on the 27^{th} day in RA patients/subjects.

FIGS. 16A and 16B show preliminary Compound 1 mean PK profile (linear scale and log-linear scale, respectively) in healthy Japanese and Chinese subjects at Day 1 (AM), after 18 mg BID administration.

FIGS. 17A and 17B show preliminary Compound 1 mean PK profile (linear scale and log-linear scale, respectively) in healthy Japanese and Chinese subjects at Day 14 (AM), after 18 mg BID administration.

FIGS. 18A and 18B show preliminary Compound 1 PK profile (mean) (linear scale and log-linear scale, respectively) in healthy Japanese subjects compared to Western Subjects.

FIGS. 19A and 19B show preliminary Compound 1 dose normalized AUC and Cₘₐₓ, respectively, in healthy Japanese subjects compared to Western Subjects.

### DETAILED DESCRIPTION OF THE INVENTION

### 1. Overview

The Jak family kinases (Jak1, Jak2, Jak3 and Tyk2) are cytoplasmic tyrosine kinases that associate with membrane bound cytokine receptors. Cytokine binding to their receptor initiates Jak kinase activation via trans and autophosphorylation processes. The activated Jak kinases phosphorylate residues on the cytokine receptors creating phosphotyrosine binding sites for SH2 domain containing proteins such as Signal Transduction Activators of Transcript (STAT) factors and other signal regulators transduction such as suppressor of cytokine signaling (SOCS) proteins and SH2 domain-containing inositol 5′-phosphatases (SHIP). Activation of STAT factors via this process leads to their dimerization, nuclear translocation and new mRNA transcription resulting in expression of immunocyte proliferation and survival factors as well as additional cytokines, chemokines and molecules that facilitate cellular trafficking (see Journal of Immunology, 2007, 178, p. 2623).

Jak kinases transduce signals for many different cytokine families and hence potentially play roles in diseases with widely different pathologies including but not limited to the following examples. Both Jak1 and Jak3 control signaling of the so-called common gamma chain cytokines (IL2, IL4, IL7, IL9, IL15 and IL21), hence simultaneous inhibition of either Jak1 or Jak3 could be predicted to impact Th1 mediated diseases such as rheumatoid arthritis via blockade of IL2, IL7 and IL15 signaling. On the other hand, IL2 signaling has recently been shown to be essential for development and homeostasis of T-regulatory cells (Malek T R et al., Immunity, 2002, 17(2), :167-178). Thus, based on genetic data, blockade of IL2 signaling alone is predicted to result in autoimmunity (Yamanouchi J et al., Nat. Genet., 2007, 39(3):329-337, and Willerford D M et al., Immunity, 1995, 3(4):521-530). Th2 mediated diseases such as asthma or atopic dermatitis via IL4 and IL9 signaling blockade. Jak1 and Tyk2 mediate signaling of IL13 (see Int. Immunity, 2000, 12:1499). Hence, blockade of these may also be predicted to have a therapeutic effect in asthma. These two kinases are also thought to mediate Type I interferon signaling; their blockade could therefore be predicted to reduce the severity of systemic lupus erythematosus (SLE). Tyk2 and Jak2 mediate signaling of IL12 and IL23. In fact, blockade of these cytokines using monoclonal antibodies has been effective in treating psoriasis. Therefore blockade of this pathway using inhibitors of these kinases could be predicted to be effective in psoriasis as well.

In summary, this invention describes small-molecule compounds that inhibit, regulate and/or modulate Jak family kinase activity that is pivotal to several mechanisms thought critical to the progression of autoimmune diseases including, but not limited to, rheumatoid arthritis (RA) such as moderate to severe RA, systemic lupus erythematosus (SLE), multiple sclerosis (MS), Crohn's disease such as moderate to severe Crohn's disease, psoriasis such as moderate to severe chronic plaque psoriasis, ulcerative colitis such as moderate to severe ulcerative colitis, ankylosing spondylitis (AS), psoriatic arthritis, Juvenile Idiopathic Arthritis (JIA) such as moderate to severe polyarticular JIA, and asthma, etc.

In particular, the compounds of the invention selectively inhibit Jak1 over the other JAK family kinases, including Jak2, Jak3, and Tyk2, and are thus expected to be efficacious against the treatable diseases while having reduced (if not eliminated) side effects resulting from inhibition of the other kinases, such as the Jak2 and/or Jak3 kinases, and signaling pathways mediated by such kinases, such as erythropoiesis and NK cell function. The invention also provides dosing regimens that, among others, provide desirable pharmacokinetic (PK) profiles of the compounds of the invention, such that Jak1 is selectively or preferentially inhibited to achieve the desired therapeutic efficacy, while at the same time, the other JAK kinases, such as Jak2 and/or Jak3, are largely or not substantially inhibited so as to avoid undesirable side effects.

Specifically, several pathologically significant cytokines signal via Jak1 alone (Guschin D, et al., EMBO J., 1995 Apr. 3, 14(7):1421-1429; Parganas E, et al., Cell, 1998 May 1, 93(3):385-395; and Rodig S. J., et al., Cell. 1998 May 1, 93(3):373-383). Blockade of one of these, IL6, using an IL6R neutralizing antibody, has been shown to significantly improve disease scores in human rheumatoid arthritis patients (Nishimoto N. et al., Ann Rheum Dis., 2007, 66(9):1162-1167). Similarly, blockaded of GCSF signaling, which is also mediated by Jak1 alone, using neutralizing monoclonal antibodies or target gene deletion protects mice from experimental arthritis (Lawlor K. E. et al., Proc. Natl. Acad. Sci. U.S.A., 2004, 101(31):11398-11403). Accordingly, the subject small-molecule compounds that selectively or preferentially inhibit, regulate and/or modulate the signal transduction of kinases, such as Jak1, is a desirable means to prevent or treat autoimmune diseases or other diseases related to aberrant Jak1 function.

Jak2 is activated in a wide variety of human cancers such as prostate, colon, ovarian and breast cancers, melanoma, leukemia and other hematopoietic malignancies. In addition, somatic point mutation of the Jak2 gene has been identified to be highly associated with classic myeloproliferative disorders (MPD) and infrequently in other myeloid disorders. Constitutive activation of Jak2 activity is also caused by chromosomal translocation in hematopoietic malignancies. It has also been shown that inhibition of the Jak/STAT pathway, and in particular inhibition of Jak2 activity, results in anti-proliferative and pro-apoptotic effects largely due to inhibition of phosphorylation of STAT. Such Jak2 blockade may lead to deficits in Erythropoietin (EPO) and Granulocyte/Monocyte-Colony Stimulating Factor (GM-CSF) signaling, and defects in erythropoiesis (see Neubauer et al., Cell 93(3):397-409, 1998). Individuals with genetic, congenital or acquired defects in these signaling pathways can develop potentially life-threatening complications such as anemia and neutrophil dysfunction.

Thus in one aspect, the invention provides a method of selectively inhibiting a Janus Kinase 1 (Jak1) in a mammal, e.g., a human, the method comprising administering to the mammal (e.g., human) an effective amount of a compound (such as the free base form of the compound), an isomer, a stereoisomer, or a pharmaceutically acceptable salt thereof, wherein less than 50%, 40%, 30%, 25%, 20%, 15%, 10%, or 5% of Jak2 and/or Jak3 activity is inhibited in the mammal (e.g., human), wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide. In certain embodiments, Jak1 activity is preferentially inhibited over activity of Jak2, activity of Jak3, and activity of Tyk2.

In a related aspect, the invention provides a method of treating in a mammal (e.g., a human) an autoimmune disease or disorder, or an inflammatory disease or disorder, the method comprising administering to the mammal (e.g., human) an effective amount of a compound (such as the free base form of the compound), an isomer, a stereoisomer, or a pharmaceutically acceptable salt thereof, wherein the effective amount is sufficient to either alleviate or inhibit the progression of a symptom of the autoimmune disease or disorder, or the inflammatory disease or disorder, wherein the effective amount reduces reticulocyte or NK cell or NKT cell or iNKT cell or CD8⁺ cell count and/or NK cell activity by no more than 50%, 40%, 30%, 25%, 20%, 15%, 10%, or 5% relative to a pre-treatment level, and, wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

In yet another related aspect, the invention provides a method of treating in a mammal (e.g., human) an autoimmune disease or disorder, or an inflammatory disease or disorder, the method comprising administering to the mammal (e.g., human) an effective amount of a compound (such as the free base form of the compound), an isomer, a stereoisomer, or a pharmaceutically acceptable salt thereof, wherein the effective amount is sufficient to either alleviate or inhibit the progression of a symptom of the autoimmune disease or disorder, or the inflammatory disease or disorder, wherein the effective amount produces an AUC₀₋₂₄ of between 0.10-1.1 μg·hr/mL (or between 0.128-1.058 μg·hr/mL) of free base equivalent of the compound, and, wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

In another related aspect, the invention provides a use of a compound (such as the free base form of the compound), an isomer, a stereoisomer, or a pharmaceutically acceptable salt thereof, in the manufacture of a medicament for selectively inhibiting a Janus Kinase 1 (Jak1) in a mammal, e.g., a human, wherein upon administration of an effective amount of the medicament to the mammal (e.g., human), less than 50%, 40%, 30%, 25%, 20%, 15%, 10%, or 5% of Jak2 and/or Jak3 activity is inhibited in the mammal (e.g., human); and wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide. In certain embodiments, Jak1 activity is preferentially inhibited over activity of Jak2, activity of Jak3, and activity of Tyk2.

In another related aspect, the invention provides a use of a compound (such as the free base form of the compound), an isomer, a stereoisomer, or a pharmaceutically acceptable salt thereof, in the manufacture of a medicament for treating in a mammal (e.g., a human) an autoimmune disease or disorder, or an inflammatory disease or disorder, wherein upon administration of an effective amount of the medicament to the mammal (e.g., human), progression of a symptom of the autoimmune disease or disorder, or the inflammatory disease or disorder, is either alleviated or inhibited; and reticulocyte or NK cell or NKT cell or iNKT cell or CD8⁺ cell count and/or NK/NKT cell activity is reduced by no more than 50%, 40%, 30%, 25%, 20%, 15%, 10%, or 5% relative to a pre-treatment level; and wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

In yet another related aspect, the invention provides a use of a compound (such as the free base form of the compound), an isomer, a stereoisomer, or a pharmaceutically acceptable salt thereof, in the manufacture of a medicament for treating in a mammal (e.g., human) an autoimmune disease or disorder, or an inflammatory disease or disorder, wherein upon administration of an effective amount of the medicament to the mammal (e.g., human), progression of a symptom of the autoimmune disease or disorder, or the inflammatory disease or disorder, is either alleviated or inhibited; and an AUC₀₋₂₄ of between 0.10-1.1 μg·hr/mL (or between 0.128-1.058 μg·hr/mL) of free base equivalent of the compound is produced and/or maintained; and wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

In another aspect, the invention provides a pharmaceutical composition for use in selectively inhibiting a Janus Kinase 1 (Jak1) in a mammal, e.g., a human, wherein upon administration of an effective amount of the pharmaceutical composition to the mammal (e.g., human), less than 50%, 40%, 30%, 25%, 20%, 15%, 10%, or 5% of Jak2 and/or Jak3 activity is inhibited in the mammal (e.g., human), and wherein the pharmaceutical composition comprises a compound (such as the free base form of the compound) of (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, an isomer, a stereoisomer, or a pharmaceutically acceptable salt thereof. In certain embodiments, Jak1 activity is preferentially inhibited over activity of Jak2, activity of Jak3, and activity of Tyk2.

In a related aspect, the invention provides a pharmaceutical composition for use in treating in a mammal (e.g., a human) an autoimmune disease or disorder, or an inflammatory disease or disorder, wherein upon administration of an effective amount of the pharmaceutical composition to the mammal (e.g., human), progression of a symptom of the autoimmune disease or disorder, or the inflammatory disease or disorder, is either alleviated or inhibited; and reticulocyte or NK cell or NKT cell or iNKT cell or CD8⁺ cell count and/or NK/NKT cell activity is reduced by no more than 50%, 40%, 30%, 25%, 20%, 15%, 10%, or 5% relative to a pre-treatment level; and wherein the pharmaceutical composition comprises a compound (such as the free base form of the compound) of (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, an isomer, a stereoisomer, or a pharmaceutically acceptable salt thereof.

In yet another related aspect, the invention provides a pharmaceutical composition for use in treating in a mammal (e.g., human) an autoimmune disease or disorder, or an inflammatory disease or disorder, wherein upon administration of an effective amount of the pharmaceutical composition to the mammal (e.g., human), progression of a symptom of the autoimmune disease or disorder, or the inflammatory disease or disorder, is either alleviated or inhibited; and an AUC₀₋₂₄ of between 0.10-1.1 μg·hr/mL (or between 0.128-1.058 μg·hr/mL) of free base equivalent of the compound is produced and/or maintained; and wherein the pharmaceutical composition comprises a compound (such as the free base form of the compound) of (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, an isomer, a stereoisomer, or a pharmaceutically acceptable salt thereof.

As used herein, the term “Natural killer cells (or NK cells)” refers to a type of cytotoxic lymphocyte critical to the innate immune system. They were named “natural killers” because of the initial notion that they do not require activation in order to kill cells that are missing “self” markers of major histocompatibility complex (MHC) class 1. NK cells (belonging to the group of Innate lymphoid cells) are defined as large granular lymphocytes (LGL) and constitute the third kind of cells differentiated from the common lymphoid progenitor generating B and T lymphocytes.

As used herein, the term “Natural killer T (or NKT) cells” refers to a heterogeneous group of T cells that share properties of both T cells and natural killer (NK) cells. Many of these cells recognize the non-polymorphic CD1d molecule, an antigen-presenting molecule that binds self- and foreign lipids and glycolipids. They constitute only approximately 0.1% of all peripheral blood T cells. The term “NKT cells” was first used in mice to define a subset of T cells that expressed the natural killer (NK) cell-associated marker NK1.1 (CD161). It is now generally accepted that the term “NKT cells” refers to CD1d-restricted T cells, present in mice and humans, some of which coexpress a heavily biased, semi-invariant T cell receptor (TCR) and NK cell markers.

NKT cells are a subset of T cells that co-express an αβ T cell receptor (TCR), but also express a variety of molecular markers that are typically associated with NK cells, such as NK1.1. The best known NKT cells differ from conventional αβ T cells in that their TCRs are far more limited in diversity (“invariant” or “Type 1” NKT). They and other CD1d-restricted T cells (“Type 2” NKT) recognize lipids and glycolipids presented by CD1d molecules, a member of the CD1 family of antigen presenting molecules, rather than peptide-MHC complexes.

NKT cells include both NK1.1⁺ and NK1.1⁻, as well as CD4⁺, CD4⁻, CD8⁺ and CD8⁻ cells. Natural Killer T cells can share other features with NK cells as well, such as CD16 and CD56 expression and granzyme production. Invariant Natural Killer T (iNKT) cells express high levels of and are dependent on the transcriptional regulator promyelocytic leukemia zinc finger (PLZF) for their development.

The best known subset of CD1d-dependent NKT cells expresses an invariant T cell receptor α (TCR-α) chain. These are referred to as type I or invariant NKT cells (iNKT) cells. These cells are conserved between humans and mice and are implicated in many immunological processes. Their TCR repertoire is usually Vα14-Jα18: Vβ8.2, 7, 2 (mouse), and Vα24-Jα18: Vβ11 (human).

NK cells differ from Natural Killer T cells (NKT) phenotypically, by origin and by respective effector functions; often NKT cell activity promotes NK cell activity by secreting IFNγ. In contrast to NKT cells, NK cells do not express T-cell antigen receptors (TCR) or Pan T marker CD3 or surface immunoglobulins (Ig) B cell receptors, but they usually express the surface markers CD16 (FcγRIII) and CD56 in humans, NK1.1 or NK1.2 in C57BL/6 mice. Up to 80% of human NK cells also express CD8.

In certain embodiments, more than 40%, 50%, 55%, 60%, 65%, 70%, 75%, 80%, 85%, 90%, 95%, 99% of Jak1 activity is inhibited in the mammal (e.g., human).

In certain embodiments, activity of Jak1 is preferentially inhibited over activity of Jak2. For example, preferential inhibition can be measured by Jak1/Jak2 potency ratio, defined as the inverse ratio of IC₅₀ of Jak1 inhibition over IC₅₀ of Jak2 inhibition. In certain embodiments, the Jak1/Jak2 potency ratio is at least about 30, 35, 40, 45, 50, 55, 60, 65, 66, 67, 68, 69, 70, 71, 72, 73, 74, 75, 76, 77, 78, 79, 80, 85, or more. In certain embodiments, the IC₅₀ of Jak1 inhibition is measured by inhibition of IL6 stimulated STAT3 phosphorylation ex vivo, for example, using a sample (e.g., a blood example) from a subject administered with Compound 1. In certain embodiments, the IC₅₀ of Jak2 inhibition is measured by inhibition of EPO stimulated STAT5 phosphorylation ex vivo, for example, using a sample (e.g., a blood example) from a subject administered with Compound 1.

In certain embodiments, activity of Jak1 is preferentially inhibited over activity of Jak3. For example, preferential inhibition can be measured by Jak1/Jak3 potency ratio, defined as the inverse ratio of IC₅₀ of Jak1 inhibition over IC₅₀ of Jak3 inhibition. In certain embodiments, the Jak1/Jak3 potency ratio is at least about 3, 5, 10, 15, 20, 25, 30, 35, 40, 45, 50, 55, 56, 57, 58, 59, 60, 65, 70 or more. In certain embodiments, the IC₅₀ of Jak1 inhibition is measured by inhibition of IL6 stimulated STAT3 phosphorylation ex vivo, for example, using a sample (e.g., a blood example) from a subject administered with Compound 1.

In certain embodiments, the mammal (e.g., human) is in need of treatment for a condition treatable by inhibition of Jak1 activity. In certain embodiments, the condition is treatable by systemic inhibition of Jak1 activity in the mammal (e.g., human). Such condition may include an inflammatory disease/disorder, or an autoimmune disease/disorder.

For example, in certain embodiments, the condition is Rheumatoid Arthritis (RA), Crohn's disease, ankylosing spondylitis (AS), psoriatic arthritis, psoriasis, ulcerative colitis, systemic lupus erythematosus (SLE), diabetic nephropathy, dry eye syndrome, Sjogren's Syndrome, organ transplant rejection, asthma, alopecia areata, vitiligo, or atopic dermatitis.

In certain embodiments, the Crohn's disease may be moderately to severely active Crohn's disease (CD) in an adult patient. In certain embodiments, the adult may be newly diagnosed of CD (e.g., having colonic or ileocolonic Crohn's disease for ≧3 months), or is inadequately responding to or has discontinued therapy due to loss of response to or intolerance to a first line therapy or an anti-TNFα therapy (e.g., azathioprine, 6-mercaptopurine (6-MP), aminosalicylate (e.g., sulfasalazine, mesalamine), corticosteroid (e.g., prednisone or prednisone equivalent, budesonide), probiotic, methotrexate, cyclosporine, tacrolimus, metronidazole, ciprofloxacin, leflunomide, chloroquine, hydroxychloroquine, penicillamine, tocilzumab, anakinra, abatacept, rituximab, efalizumab, belimumab, tofacitinib, baricitinib, golimumab, vedolizumab, natalizumab, ustekinumab, etanercept, infliximab, adalimumab, certolizumab pegol, or a JAK inhibitor). Representative Jak inhibitors include: ruxolitinib, tofacitinib or CP-690550, baricitinib (LY3009104, INCB28050), CYT387, GLPG0634, GSK2586184, lestaurtinib, pacritinib (SB1518), and TG101348.

In certain embodiments, the adult having the Crohn's disease may have an average daily liquid/very soft stool frequency score of ≧2.5 or average daily abdominal pain score of ≧2.0; and CDAI≧220 and ≦450. In certain embodiments, the adult having the Crohn's disease may have a simplified endoscopic score for Crohn's disease (SES-CD) of ≧6, or ≧4 for subjects with disease limited to the ileum.

In certain embodiments, the RA is moderately to severely active RA in an adult patient. In certain embodiments, RA-associated bone loss or bone erosion in the adult is inhibited. For example, bone loss or bone erosion may be partially inhibited such that the extent, degree, or speed of bone loss/erosion is reduced or retarded. Bone loss or bone erosion may even be completely inhibited, such that there is no further bone loss or erosion upon the commencement of treatment or shortly thereafter. In certain embodiments, bone loss or erosion may even be reversed such that there is net increase of bone mass upon the commencement of treatment or shortly thereafter. Relating to this, the method of the invention can be used to treat bone loss or bone erosion in arthritis (e.g., RA or moderately to severely active RA in an adult).

In certain embodiments, the adult may be newly diagnosed of RA, is inadequately responding to DMARDs (such as oral or biologic DMARDs), or has discontinued therapy due to loss of response to or unacceptable toxicity from methotrexate, chloroquine, azathioprine, hydroxychloroquine, penicillamine, sulfasalazine, leflunomide, tocilzumab, anakinra, abatacept, certolizumab, tofacitinib, golimumab, baricitinib, etanercept, infliximab, or adalimumab.

A salient feature of the invention is that a therapeutically effective amount of the compounds of the invention can be administered to a patient in need thereof to selectively inhibit Jak1 kinase activity (preferably systemically), without significantly inhibit or compromise the activity of the other Jak kinases, such as Jak2, Jak3, and/or Tyk2.

Thus in certain embodiments, the method of the invention does not substantially reduce (e.g., reduces no more than 50%, 45%, 40%, 35%, 30%, 25%, 20%, 15%, 10%, 5% or less) NK cell count, NKT cell count, iNKT cell count, and/or CD8⁺ cell count.

In certain embodiments, the method of the invention does not substantially inhibit (e.g., inhibits no more than 50%, 40%, 30%, 25%, 20%, 15%, 10%, 5% or less) erythropoiesis, granulocyte/monocyte-colony stimulating factor (GM-CSF) signaling, or emergency myelopoiesis in response to microbial infection in the mammal (e.g., human).

In certain embodiments, the human has anemia, or has a whole blood hemoglobin level of less then 12, 11, 10, 9, 8, 7, 6, or 5 g/dL. For example, the whole blood hemoglobin level may be measured by standard clinical laboratory methods, such as CBC (complete blood count) test from a whole blood sample.

In certain embodiments, a therapeutically effective amount of the compound (such as the free base form of the compound), isomer thereof, stereoisomer thereof, or pharmaceutically acceptable salt thereof is administered to the mammal (e.g., human) until a substantially steady level of therapeutically effective AUC₀₋₂₄ level is reached and maintained.

For example, in a human patient, an AUC₀₋₂₄ of about between 0.10-1.1 μg·hr/mL (or between 0.128-1.058 μg·hr/mL) of free base equivalent of the compound is therapeutically effective for, e.g., Rheumatoid Arthritis (moderately to severely active RA in an adult patient).

In certain embodiments, the AUC₀₋₂₄ can be achieved in an adult patient by administering the compound of the invention (e.g., the free base form of Compound 1), an isomer thereof, a stereoisomer thereof, or a pharmaceutically acceptable salt thereof, to the human twice daily (BID), preferably in equal amounts of 3-24 mg (e.g., 3, 6, 9, 12, 18, or 24 mg) of free base form equivalent of Compound 1.

In certain embodiments, the compound may be administered to the human once daily (QD), e.g., at a dose of about 18 mg or 24 mg of free base equivalent of the compound.

In certain embodiments, the AUC₀₋₂₄ is maintained at substantially the same level over a treatment period. For example, the treatment period may be at least 14 days, at least one month, 3 months, 6 months, 9 months, 1 year, 2 years, 5 years, 10 years, 20 years, 50 years etc.

In certain embodiments, inhibition of Jak1 activity while not significantly inhibiting other Jak kinases (such as Jak3) may be determined by measuring ex vivo stimulated IL-7-dependent STAT5 phosphorylation, and/or by determining peripheral NK cell or NKT cell or iNKT cell or CD8⁺ cell counts and/or NK cell activity (see below). For example, peripheral NK cell counts/activity can be measured using any art-recognized methods, such as chromium release assay and flow cytometry. Kane et al. describe a flow cytometric assay for the clinical measurement of NK cell activity (Clin. Diagn. Lab Immunol. 3(3):295-300, 1996, incorporated by reference), which may be used in the methods of the invention.

In certain embodiments, the method further comprises administering to the mammal (e.g., human) one or more additional agents which modulate a mammalian immune system or which are anti-inflammatory agents.

For example, the additional agent may be selected from the group consisting of: cyclosporin A, rapamycin, tacrolimus, deoxyspergualin, mycophenolate, daclizumab, muromonab-CD3, antithymocyte globulin, aspirin, acetaminophen, aminosalicylate, ciprofloxacin, corticosteroid, cyclosporine, metronidazole, probiotic, tacrolimus, ibuprofen, naproxen, piroxicam, prednisolone, dexamethasone, anti-inflammatory steroid, methotrexate, chloroquine, azathioprine, hydroxychloroquine, penicillamine, sulfasalazine, leflunomide, tocilzumab, anakinra, abatacept, certolizumab pegol, golimumab, vedolizumab, natalizumab, ustekinumab, rituximab, efalizumab, belimumab, etanercept, infliximab, adalimumab, or an immune modulator (e.g., activator) for CD4⁺CD25⁺ T_{reg} cells.

In certain embodiments, the method further comprises: (1) identifying a human subject administered with the compound but having inadequate or suboptimal response or therapeutic efficacy; (2) determining reticulocyte, NK cell, NKT cell, iNKT cell, and/or CD8⁺ cell count of the human subject, wherein a decrease in reticulocyte, NK cell, NKT cell, iNKT cell, or CD8⁺ cell count of no more than 30%, 25%, 20%, 15%, or 10% compared to a pre-treatment baseline level of reticulocyte, NK cell, NKT cell, iNKT cell, or CD8⁺ cell count, respectively, is indicative that the human subject is a candidate for dose escalation; (3) administering to the candidate an escalated dose of the compound. In certain embodiments, the method further comprises repeating steps (1)-(3) until a desired outcome is achieved.

In a related embodiment, the method further comprises: (1) identifying a human subject administered with the compound but having inadequate or suboptimal response or therapeutic efficacy; (2) determining reticulocyte, NK cell, NKT cell, iNKT cell, and/or CD8⁺ cell count of the human subject, wherein a decrease in reticulocyte, NK cell, NKT cell, iNKT cell, or CD8⁺ cell count of no more than 30%, 25%, 20%, 15%, or 10% compared to a pre-treatment baseline level of reticulocyte, NK cell, NKT cell, iNKT cell, or CD8⁺ cell count, respectively, is indicative that the human subject is a candidate for dose escalation; (3) administering to the candidate a second therapeutic agent. In certain embodiments, the method further comprises repeating steps (1)-(3) until a desired outcome is achieved.

In another related embodiments, the method further comprises: (1) identifying a human subject administered with the compound and is intolerable to the treatment; (2) determining reticulocyte, NK cell, NKT cell, iNKT cell, and/or CD8⁺ cell count of the human subject, wherein a decrease in reticulocyte, NK cell, NKT cell, iNKT cell, or CD8⁺ cell count of more than 30%, 35%, 40%, 45%, 50%, 55%, 60%, 65%, or 70% compared to a pre-treatment baseline level of reticulocyte, NK cell, NKT cell, iNKT cell, or CD8⁺ cell count, respectively, is indicative that the human subject is a candidate for dose reduction; (3) administering to the candidate a reduced dose of the compound. In certain embodiments, the method further comprises repeating steps (1)-(3) until a desired outcome is achieved.

The several embodiments immediately above are partly based on the surprising discovery that selective Jak1 inhibition over Jak3 leads to relatively specific inhibition of IL6-mediated (Jak1 specific) signaling to achieve excellent therapeutic efficacy, while at the same time, only creating relatively minor impact on signaling via IL7 and other common gamma chain cytokines (that depends on Jak1 and Jak3 signaling), such that undesirable side effects (such as NK cell count decrease) are largely avoided.

Specifically, signaling by IL7 and other common gamma chain cytokines (e.g., IL2, IL9, IL15, and IL21) are inhibited based on some function of the inhibition of both Jak1 and Jak3. However, prior to the instant invention, other than that inhibiting either Jak1 or Jak3 will in some way impact overall signaling efficiency, it is unclear if the function is additive or multiplicative. It was expected that potent inhibition of either Jak1 or Jak3 might severely inhibit or impair common gamma chain signaling and its downstream biological output, such as NK cell count.

Data presented herein, however, indicates that the two kinases may be less cooperative (or function somewhat independently) in the signaling complex than might have been previously thought. Given the high degree of selectivity of Compound 1 against Jak1 over Jak3 (e.g., a Jak1/Jak3 potency ratio of about 58-fold), the inhibition of common gamma chain signaling may largely reflect Jak1 inhibition. Indeed, Compound 1 potency vs. IL6 and IL7 signaling is very similar, whereas other compounds with substantial inhibitory function against Jak3 (e.g., Tofacitinib) have greater potency against common gamma chain signaling than IL6 signaling.

Thus in patients having received treatment by Compound 1 but exhibits inadequate or suboptimal therapeutic efficacy (such as not achieving a desired degree of relief or improvement in a disease symptom), or simply does not respond to treatment as others under the standard treatment regimen, a dose escalation may be considered to achieve better therapeutic efficacy, so long as the patient can tolerate the undesired side effects, such as decrease in reticulocyte, NK cell, NKT cell, iNKT cell, and/or CD8⁺ cell count. Identifying patients having inadequate response yet only minor decrease in reticulocyte, NK cell, NKT cell, iNKT cell, and/or CD8⁺ cell count may facilitate the treatment of such a patient population, who are in need of better therapeutic efficacy than the standard dose can afford, and can tolerate escalated doses.

Alternatively or in addition, a second therapeutic agent may be administered to achieve better therapeutic efficacy, since the patient is likely more able to tolerate the associated side effect than other patients who already have a relatively large decrease in reticulocyte, NK cell, NKT cell, iNKT cell, and/or CD8⁺ cell count. Any therapeutic agents contemplated to be suitable for combination therapy with the compound of the invention may be the second therapeutic agent.

Conversely, in patients having received treatment by Compound 1 but exhibits intolerable side effects, such as excessive decrease in reticulocyte, NK cell, NKT cell, iNKT cell, and/or CD8⁺ cell count, a dose reduction may be considered to relieve such intolerable side effects while maintaining therapeutic efficacy. Identifying patients having intolerable decrease in reticulocyte, NK cell, NKT cell, iNKT cell, and/or CD8⁺ cell count may facilitate the treatment of such a patient population.

Alternatively or in addition, if the patient is already under combination therapy using Compound 1 and one or more second therapeutic agents, one or more of such second therapeutic agents may be removed from the combination therapy to lessen the impact on decrease in reticulocyte, NK cell, NKT cell, iNKT cell, and/or CD8⁺ cell count.

Yet another aspect of the invention provides a pharmaceutical formulation for treating an autoimmune disease or disorder, or an inflammatory disease or disorder, the pharmaceutical composition comprising: (1) a unit dose of a compound (such as the free base form of the compound), an isomer, a stereoisomer, or a pharmaceutically acceptable salt thereof, wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, and (2) a pharmaceutically acceptable excipient, wherein the unit dose, upon administration to an adult human twice daily (BID), produces an AUC₀₋₂₄ of between 0.10-1.1 μg·hr/mL (or between 0.128-1.058 μg·hr/mL) of free base equivalent of the compound.

In a related aspect, the invention provides a pharmaceutical formulation for treating an autoimmune disease or disorder, or an inflammatory disease or disorder, the pharmaceutical composition comprising: (1) a unit dose of the free base form of the compound (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, and (2) a pharmaceutically acceptable excipient, wherein the unit dose, upon administration to an adult human twice daily (BID), preferentially inhibits activity of Jak1 over activity of Jak2, activity of Jak3, and activity of Tyk2, and inhibits less than 50%, 40%, 30%, 25%, 20%, 15%, 10%, or 5% of Jak2 and/or Jak3 activity in the human.

In another related aspect, the invention provides a pharmaceutical formulation for treating an autoimmune disease or disorder, or an inflammatory disease or disorder, the pharmaceutical composition comprising: (1) a unit dose of the free base form of the compound (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, and (2) a pharmaceutically acceptable excipient, wherein the unit dose, upon administration to an adult human twice daily (BID), reduces reticulocyte or NK cell or NKT cell or iNKT cell or CD8⁺ cell count by no more than 50%, 40%, 30%, 25%, 20%, 15%, 10%, 5% relative to a pre-treatment level.

In certain embodiments, the unit dose is a capsule, a solution, a suspension, a tablet, a pill, a sachet, a capsule, multiparticulates and a powder.

In certain embodiments, the unit dose is about 0.5, 1, 3, 6, 9, 12, 18, or 24 mg of free base equivalent of the compound.

In certain embodiments, the autoimmune disease or disorder, or inflammatory disease or disorder is Crohn's disease (e.g., moderately to severely active CD) in an adult.

In certain embodiments, the autoimmune disease or disorder, or inflammatory disease or disorder is Rheumatoid Arthritis (RA), such as moderately to severely active RA in adult patient.

In certain embodiments, the pharmaceutically acceptable excipient comprises microcrystalline cellulose, dibasic calcium phosphate, magnesium stearate, croscarmellose sodium, hydroxypropyl cellulose, or a mixture thereof.

In certain embodiments, the pharmaceutically acceptable salt is selected from the group consisting of: hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate salt. In certain embodiments, the pharmaceutically acceptable salt is tartrate salt.

In certain embodiments, the pharmaceutical formulation is formulated for oral (e.g., selective release in certain parts of the small intestine), topical, dermal, intra-luminal (e.g., via enema for GI or colon indications), or ophthalmic administration.

With the invention generally described, specific aspects of the invention are described in the sections below in further detail.

### 2. Compounds of the Invention, Isomers, Stereoisomers, and Salts Thereof

As used herein, a compound of the invention or “Compound 1” may include the free base form of the compound (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide (C₁₇H₁₉F₃N₆O), an isomer thereof, a stereoisomer thereof, or a pharmaceutically acceptable salt thereof (such as the tartrate form (C₁₇H₁₉F₃N₆O.C₄H₆O₆)).

In certain embodiments, Compound 1 refers to the free base form of (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

In certain embodiments, Compound 1 refers to the tartrate form used in certain examples described herein below. The Compound 1 tartrate is a white to light yellow powder, is sparingly soluble at pH 4.5 and slightly soluble at pH 6.8 (at 37° C. per USP criteria). The Compound 1 tartrate has 2 stereogenic centers, and is manufactured as a single stereoisomer.

In certain embodiments, Compound 1 refers to the free base form of the compound (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide (C₁₇H₁₉F₃N₆O), and does not include its enantiomer.

Certain compounds of the present invention may be basic in nature and are capable of forming a wide variety of different salts with various inorganic and organic acids. Although such salts must be pharmaceutically acceptable for administration to animals, including human, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, mono-malic acid, mono oxalic acid, tartaric acid such as mono tartaric acid (e.g., (+) or (−)-tartaric acid or mixtures thereof), amino acids (e.g., (+) or (−)-amino acids or mixtures thereof), and the like. These salts can be prepared by methods known to those skilled in the art.

Certain compounds of the invention may be provided as salts with pharmaceutically compatible counter ions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Thus the present invention also relates to the pharmaceutically acceptable acid addition salts of the compounds of the invention, such as the free base form of Compound 1, isomers and steoreoisomers thereof. The acids which are used to prepare the pharmaceutically acceptable acid addition salts are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate salts, such as 1,1′-methylene-bis-(2-hydroxy-3-naphthoate) salt.

Certain compounds of the invention and their salts may exist in more than one crystal form and the present invention includes each crystal form and mixtures thereof.

Certain compounds of the invention and their salts may also exist in the form of solvates, for example hydrates, and the present invention includes each solvate and mixtures thereof.

Certain compounds of the invention may contain one or more chiral centers, and exist in different optically active forms. In general, when compounds contain one chiral center, the compounds may exist in two enantiomeric forms and includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of the invention contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of the invention (such as Compound 1), and mixtures thereof.

Certain compounds of the invention may exist in different tautomeric forms or as different geometric isomers, and the present invention includes each tautomer and/or geometric isomer of compounds of the invention and mixtures thereof.

Certain compounds of the invention may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of the invention and mixtures thereof.

Thus the compounds of this invention include all conformational isomers (e.g., cis and trans isomers). The compounds of the present invention have asymmetric centers and therefore exist in different enantiomeric and diastereomeric forms. In certain embodiments, the invention relates to the use of all optical isomers and stereoisomers of the compounds of the present invention, and mixtures thereof, and to all pharmaceutical compositions and methods of treatment that may employ or contain them. In certain embodiments, the invention relates to the use of selected optical isomers and stereoisomers of the compounds of the present invention, such as (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide (C₁₇H₁₉F₃N₆O), and to all pharmaceutical compositions and methods of treatment that may employ or contain the selected optical isomers and stereoisomers.

The compounds of invention may also exist as tautomers. In certain embodiments, the invention relates to the use of all such tautomers and mixtures thereof.

Certain compounds of the invention may exist in zwitterionic form and the present invention includes each zwitterionic form of compounds of the invention and mixtures thereof.

The synthesis of the compounds of the invention, including (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, pharmaceutically acceptable salts thereof, stereoisomers thereof, and isomers thereof, is provided in U.S. Pat. No. 8,426,411, the entire content of which is incorporated herein by reference.

For example, (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide can be synthesized according to the following scheme:

### N-Alkylation Using Alkyl Halide, α-Haloketone or α-Haloamide

A round bottom flask is charged with a base such as NaH (60% dispersion in mineral oil), K₂CO₃, or Cs₂CO₃ (preferably NaH (60% dispersion in mineral oil), 0.9-1.5 equiv., preferably 0.95 equiv.) and an organic solvent (such as N, N-dimethylformamide (DMF), dichloromethane (DCM), 1,4-dioxane, or N-methyl-2-pyrrolidone (NMP), preferably DMF). The mixture is cooled to about −10° C. to ambient temperature (preferably about 0° C.) and a solution of an appropriately substituted amine (preferably 1 equiv.) in an organic solvent (such as DMF) is added. Alternatively, the base may be added portionwise to a solution of the amine and an organic solvent at about 0° C. to ambient temperature. The reaction mixture is stirred for about 5-90 min (preferably about 15-30 min) at about −10° C. to ambient temperature (preferably about 0° C.) followed by the addition of an alkyl halide, α-haloketone, or α-haloamide (1-2 equiv., preferably 1.2 equiv.). Alternatively, a solution of an amine and a base in an organic solvent may be added to a solution of an alkyl halide, α-haloketone, or α-haloamide in an organic solvent at about 0° C. The reaction mixture is stirred at about −10° C. to ambient temperature (preferably ambient temperature) for about 0.5-24 h (preferably about 1 h). Optionally, the organic solvent may be removed under reduced pressure. Optionally, the reaction mixture or residue may be diluted with water, aqueous NH₄Cl, or aqueous NaHCO₃. If a precipitate forms the solid may be optionally collected via vacuum filtration to give the target compound. Alternatively, an organic solvent (such as ethyl acetate (EtOAc) or DCM) is added to the aqueous mixture and the layers are separated. The aqueous layer may optionally be extracted further with an organic solvent (such as EtOAc and/or DCM). The combined organic layers are optionally washed with additional aqueous solutions such as brine, dried over anhydrous Na₂SO₄ or MgSO₄, filtered, and concentrated to dryness under reduced pressure.

The procedure above is illustrated below in the preparation of tert-butyl 2-amino-2-oxoethyl(5-tosyl-5H-pyrrolo[3,2-b]pyrazin-2-yl)carbamate from tert-butyl (5-tosyl-5H-pyrrolo[2,3-b]pyrazin-2-yl)carbamate.

To a solution of tert-butyl 5-tosyl-5H-pyrrolo[3,2-b]pyrazin-2-ylcarbamate (1.00 g, 2.57 mmol, Example #3 Step E) and DMF (13 mL) under nitrogen at about 0° C. was added NaH (60% dispersion in mineral oil, 0.113 g, 2.83 mmol) in one portion. After about 30 min, 2-bromoacetamide (0.391 g, 2.83 mmol) was added in one portion. After about 30 min, the ice bath was removed and the solution was stirred at ambient temperature for about 2 h. Saturated aqueous NH₄Cl/water (1:1, 100 mL) was added. After stirring for about 10 min, the mixture was filtered using water to wash the filter cake. The aqueous phase was extracted with EtOAc (50 mL). The filter cake was dissolved in EtOAc and added to the organic layer. The organic layer was dried over Na₂SO₄, filtered, and concentrated under reduced pressure. The material was purified by silica gel chromatography eluting with a gradient of 20-100% EtOAc/heptane to give tert-butyl 2-amino-2-oxoethyl(5-tosyl-5H-pyrrolo[3,2-b]pyrazin-2-yl)carbamate (0.980 g, 82%): LC/MS (Table 1, Method n) R_{z}=0.70 min; MS m/z 446 (M+H)⁺.

Similar reaction condition can also be used to synthesize benzyl 3-ethyl-4-(2-((5-tosyl-5H-pyrrolo[2,3-b]pyrazin-2-yl)amino)acetyl)pyrrolidine-1-carboxylate from tert-butyl (5-tosyl-5H-pyrrolo[2,3-b]pyrazin-2-yl)carbamate and benzyl 3-(2-bromoacetyl)-4-ethylpyrrolidine-1-carboxylate.

### Cyclization of a ketone using a dithiaphosphetane reagent (e.g., synthesizing (3S,4R)-benzyl 3-ethyl-4-(3-tosyl-3H-imidazo[1,2-a]pyrrolo[2,3-e]pyrazin-8-yl)pyrrolidine-1-carboxylate from benzyl 3-ethyl-4-(2-((5-tosyl-5H-pyrrolo[2,3-b]pyrazin-2-yl)amino)acetyl)pyrrolidine-1-carboxylate)

To a solution of a ketone (preferably 1 equiv.) in an organic solvent such as tetrahydrofuran (THF) or 1,4-dioxane (preferably 1,4-dioxane) is added a thiolating reagent such as Lawesson's reagent or Belleau's reagent (2,4-bis(4-phenoxyphenyl)-1,3-dithia-2,4-diphosphetane-2,4-disulfide) (0.5-2.0 equiv., preferably Lawesson's reagent, 0.5-0.6 equiv.). The reaction is heated at about 30° C. to 120° C. (preferably about 60-70° C.) for about 0.5-10 h (preferably about 1-2 h). Optionally, additional thiolating reagent (0.5-2.0 equiv., preferably 0.5-0.6 equiv.) can be added to the reaction mixture and heating can be continued for about 0.5-10 h (preferably about 1-2 h). The reaction mixture is concentrated under reduced pressure.

### Preparation of 8-((cis)-4-ethylpyrrolidin-3-yl)-3-tosyl-3H-imidazo[1,2-a]pyrrolo[2,3-e]pyrazine from (3S,4R)-benzyl 3-ethyl-4-(3-tosyl-3H-imidazo[1,2-a]pyrrolo[2,3-e]pyrazin-8-yl)pyrrolidine-1-carboxylate

To a solution of (cis)-benzyl 3-ethyl-4-(3-tosyl-3H-imidazo[1,2-a]pyrrolo[2,3-e]pyrazin-8-yl)pyrrolidine-1-carboxylate (0.838 g, 1.541 mmol) is added a solution of HBr (2.50 mL, 15.19 mmol, 33% in acetic acid). The reaction mixture is stirred at ambient temperature for about 1 h. The reaction is diluted with diethyl ether or Et₂O (50 mL) and water (20 mL). The layers are stirred for about 3 min and the organic layer is decanted then the procedure is repeated 5 times. The aqueous layer is cooled to about 0° C. and is basified with saturated aqueous NaHCO₃ solution (10 mL) to about pH 7. The aqueous layer is extracted with EtOAc (3×50 mL), combined, and dried over anhydrous Na₂SO₄, filtered and concentrated to give a brown solid. The solid is dissolved in DCM (50 mL) and washed with water (3×20 mL), dried over anhydrous Na₂SO₄, filtered and concentrated to afford 8-((cis)-4-ethylpyrrolidin-3-yl)-3-tosyl-3H-imidazo[1,2-a]pyrrolo[2,3-e]pyrazine (0.453, 61%) as a brown residue: LC/MS (Table 1, Method a) Rₜ=1.73 min; MS m/z: 410 (M+H)⁺.

### Hydrolysis of a sulfonamide (e.g., 8-((3R,4S)-4-ethylpyrrolidin-3-yl)-3-tosyl-3H-imidazo[1,2-a]pyrrolo[2,3-e]pyrazine to 8-((3R,4S)-4-ethylpyrrolidin-3-yl)-3H-imidazo[1,2-a]pyrrolo[2,3-e]pyrazine)

To a flask containing a sulfonamide, for example, a sulfonyl-protected pyrrole, (preferably 1 equiv.) in an organic solvent (such as 1,4-dioxane, methanol (MeOH), or THF/MeOH, preferably 1,4-dioxane) is added an aqueous base (such as aqueous Na₂CO₃ or aqueous NaOH, 1-30 equiv., preferably 2-3 equiv. for aqueous NaOH, preferably 15-20 equiv. for aqueous Na₂CO₃). The mixture is stirred at about 25-100° C. (preferably about 60° C.) for about 1-72 h (preferably about 1-16 h). In cases where the reaction does not proceed to completion as monitored by TLC, LC/MS, or HPLC, additional aqueous base (such as aqueous Na₂CO₃, 10-20 equiv., preferably 10 equiv. or aqueous NaOH, 1-5 equiv., preferably 1-2 equiv.) and/or a cosolvent (such as ethanol (EtOH)) is added. The reaction is continued at about 25-100° C. (preferably about 60° C.) for about 0.25-3 h (preferably about 1-2 h). In any case where an additional base labile group is present (for example, an ester a trifluoromethyl, or a cyano group), this group may also be hydrolyzed. The reaction is worked up using one of the following methods. Method 1. The organic solvent is optionally removed under reduced pressure and the aqueous solution is neutralized with the addition of a suitable aqueous acid (such as aqueous HCl). A suitable organic solvent (such as EtOAc or DCM) and water are added, the layers are separated, and the organic solution is dried over anhydrous Na₂SO₄ or MgSO₄, filtered, and concentrated to dryness under reduced pressure to give the target compound. Method 2. The organic solvent is optionally removed under reduced pressure, a suitable organic solvent (such as EtOAc or DCM) and water are added, the layers are separated, and the organic solution is dried over anhydrous Na₂SO₄ or MgSO₄, filtered, and concentrated to dryness under reduced pressure to give the target compound. Method 3. The reaction mixture is concentrated under reduced pressure and directly purified by one of the subsequent methods.

### Formation of a urea using CDI or thiocarbonyldiimidazole, respectively (e.g., from 8-((3R,4S)-4-ethylpyrrolidin-3-yl)-3H-imidazo[1,2-a]pyrrolo[2,3-e]pyrazine to (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide)

To a solution or slurry of an amine or amine salt (1-3 equiv., preferably 1-2 equiv.) in an organic solvent such as DCM, THF, or DMF (preferably DMF) at about 20-80° C. (preferably about 65° C.) is optionally added an organic base, such as triethylamine (TEA), N,N-diisopropylethylamine (DIEA), pyridine (preferably TEA) (1-10 equiv., preferably 1-5 equiv.) followed by CDI or 1,1′-thiocarbonyldiimidazole (0.5-2 equiv., preferably 1 equiv.). After about 0.5-24 h (preferably about 1-3 h), a second amine or amine salt (1-10 equiv., preferably 1-3 equiv.) is added neat or as a solution or slurry in an organic solvent such as DCM, THF, or DMF (preferably DMF). The reaction is held at about 20-80° C. (preferably about 65° C.) for about 2-24 h (preferably about 3 h). If the reaction mixture is heated, it is cooled to ambient temperature. The reaction mixture is partitioned between an organic solvent (such as EtOAc, DCM or 1,4-dioxane) and an aqueous base (such as saturated aqueous NaHCO₃ or saturated aqueous Na₂CO₃, preferably saturated aqueous NaHCO₃). Optionally, the reaction mixture is concentrated under reduced pressure and the residue is partitioned as above. In either case, the aqueous layer is then optionally extracted with additional organic solvent such as EtOAc or DCM. The combined organic layers may optionally be washed with brine and concentrated in vacuo or dried over anhydrous Na₂SO₄ or MgSO₄ and then decanted or filtered prior to concentrating under reduced pressure to give the target compound. Optionally, the reaction mixture is concentrated under reduced pressure and the residue is directly purified.

### Chiral Preparative HPLC Purification

Chiral purification is performed using Varian 218 LC pumps, a Varian CVM 500 with switching valves and heaters for automatic solvent, column and temperature control and a Varian 701 Fraction collector. Detection methods include a Varian 210 variable wavelength detector, an in-line polarimeter (PDR-chiral advanced laser polarimeter, model ALP2002) used to measure qualitative optical rotation (+/−) and an evaporative light scattering detector (ELSD) (a PS-ELS 2100 (Polymer Laboratories)) using a 100:1 split flow. ELSD settings are as follows: evaporator: 46° C., nebulizer: 24° C. and gas flow: 1.1 SLM. The absolute stereochemistry of the purified compounds was assigned arbitrarily and is drawn as such. Compounds of the invention where the absolute stereochemistry has been determined by the use of a commercially available enantiomerically pure starting material, or a stereochemically defined intermediate, or X-ray diffraction are denoted by an asterisk after the example number.

(cis)-3-ethyl-4-(3H-imidazo[1,2-a]pyrrolo[2,3-e]pyrazin-8-yl)-N-(2,2,2-trifluoroethyl)pyrrolidine-1-carboxamide isolated using the above method has an Rₜ min of 1.52, and m/z ESI+ (M+H)⁺ of 381.

The starting materials and intermediates of the above synthesis scheme may be obtained using the following schemes:

### Preparation of starting material of 1-(tert-butoxycarbonyl)-4-ethylpyrrolidine-3-carboxylic acid

### Step A: ethyl pent-2-ynoate to (Z)-ethyl pent-2-enoate

To a slurry of Lindlar catalyst (0.844 g, 0.396 mmol) in THF (100 mL) and pyridine (10.00 mL) is added ethyl pent-2-ynoate (5.22 mL, 39.6 mmol). The reaction mixture is sparged with hydrogen for about 10 min and an atmosphere of hydrogen is maintained via balloon. After about 15 h the reaction mixture is filtered through a pad of Celite®, diluted with Et₂O (30 mL) and washed with saturated aqueous CuSO₄ (40 mL), followed by water (40 mL). The organic layer is separated, dried over anhydrous MgSO₄, filtered, and concentrated in vacuo to provide crude (Z)-ethyl pent-2-enoate (5 g, 98%). ¹H NMR (DMSO-d₆) δ 1.05 (t, 3H), 1.28 (t, 3H), 2.65 (m, 2H), 4.18 (q, 2H), 5.72 (m, 1H), 6.21 (m, 1H).

### Step B: (cis)-ethyl 1-benzyl-4-ethylpyrrolidine-3-carboxylate (from (Z)-ethyl pent-2-enoate and N-benzyl-1-methoxy-N-((trimethylsilyl)methyl)methanamine)

To a solution of N-benzyl-1-methoxy-N-((trimethylsilyl)methyl)methanamine (9.98 mL, 39.0 mmol) and (4-ethyl pent-2-enoate (5 g, 39.0 mmol) in DCM (50 mL) is added trifluoroacetic acid (TFA) (0.030 mL, 0.390 mmol) at RT. After about 2 days, the reaction mixture is concentrated in vacuo to provide crude (cis)-ethyl 1-benzyl-4-ethylpyrrolidine-3-carboxylate (9.8 g, 96%) as an oil. LC/MS (Table 1, Method a) Rₜ=1.62 min; MS m/z: 262 (M+H)⁺.

### Step C: ethyl 1-benzyl-4-ethylpyrrolidine-3-carboxylate to (cis)-ethyl 4-ethylpyrrolidine-3-carboxylate

A Parr shaker is charged with PdOH₂ on carbon (2.243 g, 3.19 mmol) and (cis)-ethyl 1-benzyl-4-ethylpyrrolidine-3-carboxylate (16.7 g, 63.9 mmol) followed by EtOH (100 mL). The reaction mixture is degassed and purged with hydrogen gas and shaken on the parr shaker at 60 psi for about 4 days at ambient temperature. The reaction mixture is degassed and purged with nitrogen. The suspension is filtered through a pad of Celite® washing with EtOH (˜900 mL). The solvent is removed under reduced pressure to afford (cis)-ethyl 4-ethylpyrrolidine-3-carboxylate (8.69 g, 79%) as an oil: LC/MS (Table 1, Method a) Rₜ=1.11 min; MS m/z: 172 (M+H)⁺.

### Step D: (cis)-ethyl 4-ethylpyrrolidine-3-carboxylate to (cis)-1-(tert-butoxycarbonyl)-4-ethylpyrrolidine-3-carboxylic acid

To a flask charged with (cis)-ethyl 4-ethylpyrrolidine-3-carboxylate (8.69 g, 50.7 mmol) is added aqueous HCl (6N, 130 mL, 782 mmol). The solution is heated at about 75° C. for about 12 h. aqueous HCl (6N, 100 mL, 599 mmol) is added and stirred at about 80° C. for about 20 h. Aqueous HCl (6N, 100 mL, 599 mmol) is added and continued stirring at about 80° C. for about 20 h. The reaction mixture is cooled to ambient temperature and the solvent is removed under reduced pressure. 1,4-Dioxane (275 mL) and water (50 mL) are added followed by portionwise addition of Na₂CO₃ (13.5 g, 127 mmol). Di-tert-butyl dicarbonate (13.3 g, 60.9 mmol) is added and the reaction mixture is stirred at ambient temperature for about 16 h. The solid is filtered and washed with EtOAc (250 mL). The aqueous layer is acidified with aqueous HCl (1N) to about pH 3-4. The layers are partitioned and the aqueous layer is extracted with EtOAc (3×100 mL). The combined organic layers are dried over anhydrous Na₂SO₄, filtered and removed under reduced pressure. As the organic layer is almost fully concentrated (˜10 mL remaining), a solid precipitated. Heptane (30 mL) is added and the solid is filtered washing with heptane to afford (cis)-1-(tert-butoxycarbonyl)-4-ethylpyrrolidine-3-carboxylic acid (3.9 g, 32%) as an off white solid as product: LC/MS (Table 1, Method c) Rₜ=0.57 min; MS m/z: 242 (M−H)⁻.

### Synthesis of Intermediate benzyl 3-(2-bromoacetyl)-4-ethylpyrrolidine-1-carboxylate Acidic cleavage of a Boc-protected amine (e.g., 1-(tert-butoxycarbonyl)-4-ethylpyrrolidine-3-carboxylic acid to 4-ethylpyrrolidine-3-carboxylic acid hydrochloride)

To a solution of a Boc-protected amine (preferably 1 equiv.) in an organic solvent (such as DCM, 1,4-dioxane, or MeOH) is added TFA or HCl (preferably 4 N HCl in 1,4-dioxane, 2-35 equiv., preferably 2-15 equiv.). The reaction is stirred at about 20-100° C. (preferably ambient temperature to about 60° C.) for about 1-24 h (preferably about 1-6 h). In any case where an additional acid labile group is present (for example, a t-butyl ester), this group may also be cleaved during the reaction. Optionally, additional TFA or HCl (preferably 4 N HCl in 1,4-dioxane solution, 2-35 equiv., preferably 2-15 equiv.) may be added to the reaction mixture in cases where the reaction does not proceed to completion as monitored by TLC, LC/MS, or HPLC. Once the reaction has proceeded to an acceptable level, the reaction mixture can be concentrated in vacuo to provide the amine as a salt. Alternatively, the reaction may be partitioned between an organic solvent (such as EtOAc, DCM or 1,4-dioxane) and an aqueous base (such as saturated aqueous NaHCO₃ or saturated aqueous Na₂CO₃, preferably saturated aqueous NaHCO₃). The aqueous layer can be optionally extracted with additional organic solvent such as EtOAc or DCM. The combined organic layers may optionally be washed with brine, dried over anhydrous Na₂SO₄ or MgSO₄, then decanted or filtered, prior to concentrating under reduced pressure to give the target compound.

### Cbz-protection of an amine (e.g., 4-ethylpyrrolidine-3-carboxylic acid hydrochloride to 1-((benzyloxy)carbonyl)-4-ethylpyrrolidine-3-carboxylic acid)

A solution of an amine or an amine salt (preferably 1 equiv.) and a base (for example, Na₂CO₃ or NaOH, 1-3 equiv., preferably Na₂CO₃, 1.6 equiv.) in water or aqueous organic solvent (for example, water/1,4-dioxane or water/acetonitrile (MeCN), preferably water/1,4-dioxane) is stirred at ambient temperature for about 1-10 min (preferably 5 min). A solution of benzyl 2,5-dioxopyrrolidin-1-yl carbonate (1-2 equiv., preferably 1.0 equiv.) in an organic solvent such as 1,4-dioxane or MeCN is added to the reaction. The reaction is stirred at ambient temperature for about 8-144 h (preferably about 72 h). Optionally, the reaction mixture is concentrated under reduced pressure. The resulting aqueous solution is diluted with an organic solvent (such as EtOAc or DCM). The organic extracts are optionally washed with water and/or brine, dried over anhydrous Na₂SO₄ or MgSO₄, filtered or decanted, and concentrated under reduced pressure. Alternatively, the resulting aqueous solution is acidified by adding an acid such as aqueous NH₄Cl or HCl and is then extracted with an organic solvent (such as EtOAc or DCM).

### Formation of a bromomethyl ketone from an acid (e.g., 1-((benzyloxy)carbonyl)-4-ethylpyrrolidine-3-carboxylic acid to benzyl 3-(2-bromoacetyl)-4-ethylpyrrolidine-1-carboxylate)

To a solution of a carboxylic acid (preferably 1 equiv.) in an organic solvent (DCM or 1,2-dichloroethane (DCE), preferably DCM) is slowly added oxalyl chloride (1.2-3.0 equiv., preferably 2.2 equiv.) followed by dropwise addition of DMF (0.01-0.20 equiv., preferably about 0.15 equiv.). The reaction is stirred at about 0-40° C. (preferably ambient temperature) for about 3-24 h (preferably about 14 h) before it is concentrated under reduced pressure to a constant weight to give the crude acid chloride. A solution of a crude acid chloride (preferably 1 equiv.) in an organic solvent (such as THF, MeCN, Et₂O, or THF/MeCN, preferably THF/MeCN) is added to trimethylsilyldiazomethane (2.0 M in Et₂O) or diazomethane solution in Et₂O (prepared from DIAZALD® according to Aldrich protocol or J. Chromatogr. Sci. 1991, 29:8) (2-10 equiv., preferably 3.5 equiv. of trimethylsilyldiazomethane) at about −20-20° C. (preferably about 0° C.) in a suitable organic solvent such as THF, MeCN, Et₂O, or THF/MeCN (preferably THF/MeCN). The reaction mixture is stirred for about 0.5-5 h (preferably about 3 h) at about −20-20° C. (preferably about 0° C.) before the dropwise addition of 48% aqueous HBr (5-40 equiv., preferably about 10 equiv.). After about 0-30 min, (preferably about 5 min) the reaction mixture can be concentrated to dryness to give the desired product, neutralized by a dropwise addition of saturated aqueous NaHCO₃ or is optionally washed with brine after optional addition of an organic solvent (such as EtOAc or DCM, preferably EtOAc). In cases where the reaction mixture is subjected to an aqueous work-up, the organic layer is dried over anhydrous Na₂SO₄ or MgSO₄ (preferably MgSO₄), filtered, and concentrated under reduced pressure.

### Synthesis of Intermediate tert-butyl (5-tosyl-5H-pyrrolo[2,3-b]pyrazin-2-yl)carbamate Step A: 3,5-dibromopyrazin-2-amine to 5-bromo-3-((trimethylsilyl)ethynyl)pyrazin-2-amine

To a solution of 3,5-dibromopyrazin-2-amine (125 g, 494 mmol), TEA (207.0 mL, 1483 mmol), and copper (I) iodide (0.941 g, 4.94 mmol) in THF (1255 mL) is added PdCl₂(PPh₃)₂ (3.47 g, 4.94 mmol). The reaction mixture is cooled at about −5-0° C. and a solution of (trimethylsilyl)acetylene (65.0 mL, 470 mmol) in THF (157 mL) is added dropwise over about 15 min. The reaction mixture is stirred at about −5-0° C. for about 1.5 h and then allowed to warm to room temperature (RT) overnight. The reaction mixture is then filtered through a CELITE® pad and washed with THF until no further product eluted. The filtrate is concentrated under reduced pressure to give a brown-orange solid. The solid is triturated and sonicated with warm petroleum ether (b.p. 30-60° C., 400 mL), cooled to RT, collected, washed with petroleum ether (b.p. 30-60° C.; 2×60 mL), and dried to give 5-bromo-3-((trimethylsilyl)ethynyl)pyrazin-2-amine (124 g, 93%, 93% purity) as a brown solid: LC/MS (Table 1, Method b) Rₜ=2.51 min; MS m/z: 270, 272 (M+H)⁺.

### Step B: 5-bromo-3-((trimethylsilyl)ethynyl)pyrazin-2-amine to 2-bromo-5-tosyl-5H-pyrrolo[2,3-b]pyrazine

To a solution of 5-bromo-3-((trimethylsilyl)ethynyl)pyrazin-2-amine (3.00 g, 11.1 mmol) in DMF (60 mL) at about 0° C. is added NaH (60% dispersion in mineral oil, 0.577 g, 14.4 mmol) in three portions. After about 15 min, p-toluenesulfonyl chloride (2.75 g, 14.4 mmol) is added and the reaction is allowed to warm slowly to ambient temperature. After about 16 h, the reaction mixture is poured onto ice-cold water (120 mL) and the precipitate is collected by vacuum filtration. The crude solid is dissolved in DCM (15 mL) and purified by silica gel chromatography eluting with DCM to give 2-bromo-5-tosyl-5H-pyrrolo[2,3-b]pyrazine (2.16 g, 52%): LC/MS (Table 1, Method c) Rₜ=1.58 min; MS m/z: 352, 354 (M+H)⁺.

### Step C: 2-bromo-5-tosyl-5H-pyrrolo[2,3-b]pyrazine to methyl 5-tosyl-5H-pyrrolo[2,3-b]pyrazine-2-carboxylate

CO is bubbled into an orange solution of 2-bromo-5-tosyl-5H-pyrrolo[2,3-b]pyrazine (50.0 g, 142 mmol) in DMF (2.50 L) within a 5 L round bottom flask for about 2 min. Bis(triphenylphosphine)-palladium(II) dichloride (9.96 g, 14.2 mmol), TEA (59 mL, 423 mmol) and MeOH (173.0 mL, 4259 mmol) are added and the flask is fitted with a balloon of CO. The mixture is heated at about 95° C. under an atmosphere of CO (1 atmosphere). After stirring overnight, the reaction mixture is cooled to ambient temperature overnight and poured into ice water (3.2 L). The mixture is stirred for about 10 min and the precipitate is collected by filtration, while washing with water, and dried for 1 h. The crude material is dissolved in DCM, separated from residual water, dried over anhydrous MgSO₄, filtered, added silica gel, and concentrated under reduced pressure to prepare for chromatography. The crude material is purified by silica gel column chromatography eluting with 0-5% MeOH in DCM to yield methyl 5-tosyl-5H-pyrrolo[2,3-b]pyrazine-2-carboxylate with 5 mol % DCM as an excipient (40.7 g, 86%, 93% purity): LC/MS (Table 1, Method a) Rₜ=2.35 min; MS m/z 332 (M+H)⁺.

### Step D: methyl 5-tosyl-5H-pyrrolo[2,3-b]pyrazine-2-carboxylate to 5-tosyl-5H-pyrrolo[2,3-b]pyrazine-2-carboxylic acid

HCl (6 N aqueous, 714 mL) is added to a yellow solution of methyl 5-tosyl-5H-pyrrolo[2,3-b]pyrazine-2-carboxylate (17.8 g, 53.6 mmol) in 1,4-dioxane (715 mL) within a 2 L round bottom flask, and the mixture is heated at about 60° C. for about 16 h. The reaction mixture is cooled to ambient temperature. The organic solvent is removed under reduced pressure and the precipitate is collected, washed with water, and dried to yield 5-tosyl-5H-pyrrolo[2,3-b]pyrazine-2-carboxylic acid (14.4 g, 85%) as a yellow solid: LC/MS (Table 1, Method a) Rₜ=1.63 min; MS m/z 316 (M−H)⁻.

### Step E: 5-tosyl-5H-pyrrolo[2,3-b]pyrazine-2-carboxylic acid to tert-butyl 5-tosyl-5H-pyrrolo[2,3-b]pyrazin-2-ylcarbamate

In a 500 mL round bottom flask, 5-tosyl-5H-pyrrolo[2,3-b]pyrazine-2-carboxylic acid (14.4 g, 45.3 mmol), diphenylphosphoryl azide (9.78 mL, 45.3 mmol) and TEA (13.9 mL, 100 mmol) in tert-butanol (t-BuOH) (200 mL) are added to give an orange suspension. The mixture is heated at about 70° C. for about 16 h, cooled to ambient temperature and the insoluble material is removed by filtration. The solvent is removed under reduced pressure and the crude material is purified by silica gel column chromatography eluting with 25-60% EtOAc in heptane to yield tert-butyl 5-tosyl-5H-pyrrolo[2,3-b]pyrazin-2-ylcarbamate (9.75 g, 54%) as an off-white solid: LC/MS (Table 1, Method a) Rₜ=2.79 min; MS m/z 389 (M+H)⁺.

### 3. Treatable Diseases

Jak1 is a tyrosine kinase essential for signaling for certain type I and type II cytokines. It interacts with the common gamma chain (γc) of type I cytokine receptors to elicit signals from the IL-2 receptor family (e.g., IL-2R, IL-7R, IL-9R and IL-15R), the IL-4 receptor family (e.g., IL-4R and IL-13R), and the gp130 receptor family (e.g., IL-6R, IL-11R, LIF-R, OSM-R, cardiotrophin-1 receptor (CT-1R), ciliary neurotrophic factor receptor (CNTF-R), neurotrophin-1 receptor (NNT-1R) and Leptin-R). It is also important for transducing a signal by type I (IFN-α/(β) and type II (IFN-γ) interferons, and members of the IL-10 family via type II cytokine receptors. Thus Jak1 plays a critical role in initiating responses to multiple major cytokine receptor families.

Loss of Jak1 is lethal in neonatal mice, possibly due to difficulties suckling (Rodig et al., Cell 93(3):373-383, 1998). Expression of Jak1 in cancer cells enables individual cells to contract, potentially allowing them to escape their tumor and metastasize to other parts of the body.

The compounds of the invention are selective inhibitors for Jak1 kinase involved in cellular signaling pathways implicated in numerous pathogenic conditions, including immunomodulation, inflammation, or proliferative disorders such as cancer. Thus the compounds of the invention may be used to treat such pathogenic conditions, including alleviating at least one symptom of the pathogenic conditions in which Jak1 activity is detrimental, and/or inhibiting the progression of at least one symptom or indicator of the pathogenic conditions in which Jak1 activity is detrimental.

For example, many autoimmune diseases and disease associated with chronic inflammation, as well as acute responses, have been linked to excessive or unregulated production or activity of one or more cytokines, the signaling of which depend on JAK kinases. Such diseases include rheumatoid arthritis (RA) such as moderate to severe RA, systemic lupus erythematosus (SLE), multiple sclerosis (MS), Crohn's disease such as moderate to severe Crohn's disease, psoriasis such as moderate to severe chronic plaque psoriasis, ulcerative colitis such as moderate to severe ulcerative colitis, ankylosing spondilytis (AS), psoriatic arthritis, Juvenile Idiopathic Arthritis (JIA) such as moderate to severe polyarticular JIA, systemic lupus erythematosus (SLE), diabetic nephropathy, dry eye syndrome, Sjogren's Syndrome, alopecia areata, vitiligo, or atopic dermatitis.

Treatment of these diseases or conditions, or alleviation of at least one symptoms of the diseases or conditions, may be measured by one or more art-recognized therapeutic efficacy measurements, which are described in more details in later part of this section for several exemplary treatable diseases or conditions, such as RA, JIA, Crohn's Disease, Psoriatic Arthritis, Psoriasis, Ulcerative Colitis, and Ankylosing Spondylitis.

Thus the present invention relates to a pharmaceutical composition for (a) treating or preventing a disorder or condition selected from the group consisting of: rheumatoid arthritis, multiple sclerosis, experimental allergic encephalomyelitis, lupus, Crohn's disease, vasculitis, cardiomyopathy, psoriasis, Reiter's syndrome, glomerulonephritis, ulcerative colitis, allergic asthma, insulin-dependent diabetes, peripheral neuropathy, uveitis, fibrosing alveolitis, type I diabetes, juvenile diabetes, juvenile arthritis, Castleman disease, neutropenia, endometriosis, autoimmune thyroid disease, sperm and testicular autoimmunity, scleroderma, axonal & neuronal neuropathies, allergic rhinitis, Sjogren's syndrome, hemolytic anemia, Graves' disease, Hashimoto's thyroiditis, IgA nephropathy, amyloidosis, ankylosing spondylitis, Behcet's disease, sarcoidosis, vesiculobullous dermatosis, myositis, primary biliary cirrhosis, polymyalgia rheumatica, autoimmune immunodeficiency, Chagas disease, Kawasaki syndrome, psoriatic arthritis, celiac sprue, myasthenia gravis, autoimmune myocarditis, POEMS syndrome, and chronic fatigue syndrome.

In certain embodiments, the present invention relates to a pharmaceutical composition for (a) treating or preventing a disorder or condition selected from the group consisting of: lupus (SLE), multiple sclerosis, rheumatoid arthritis, psoriasis, Type I diabetes and complications from diabetes (such as diabetic nephropathy), atopic dermatitis, autoimmune thyroid disorders, ulcerative colitis, Crohn's disease, and other autoimmune diseases or (b) the inhibition of protein kinases or Janus Kinase 1 (JAK1) in a mammal, including a human, comprising an amount of a compound of the invention (e.g., Compound 1) or a pharmaceutically acceptable salt thereof, effective in such disorders or conditions and a pharmaceutically acceptable carrier.

The present invention also relates to a method for the inhibition of protein typrosine kinases or Janus Kinase 1 (JAK1) in a mammal, including a human, comprising administering to said mammal an effective amount of a compound of the invention (e.g., Compound 1) or a pharmaceutically acceptable salt thereof.

The present invention also relates to a method for treating or preventing a disorder or condition selected from multiple sclerosis, rheumatoid arthritis, psoriasis, asthma, atopic dermatitis, autoimmune thyroid disorders, ulcerative colitis, Crohn's disease, and other autoimmune diseases in a mammal, including a human, comprising administering to said mammal an amount of a compound of the compound of the invention (e.g., Compound 1) or a pharmaceutically acceptable salt thereof, effective in treating such a condition.

The compounds of the invention are also useful in the treatment of an ocular condition, systemic inflammatory response syndrome, systemic onset juvenile rheumatoid arthritis, type III hypersensitivity reactions, type IV hypersensitivity, inflammation of the aorta, iridocyclitis/uveitis/optic neuritis, juvenile spinal muscular atrophy, diabetic retinopathy or microangiopathy, chronic inflammation, ulcerative colitis, inflammatory bowel disease, allergic diseases, dermatitis scleroderma, acute or chronic immune disease associated with organ transplantation, psoriatic arthropathy, ulcerative colitic arthropathy, autoimmune bullous disease, autoimmune haemolytic anaemia, rheumatoid arthritis associated interstitial lung disease, systemic lupus erythematosus associated lung disease, dermatomyositis/polymyositis associated lung disease, Sjogren's Syndrome/disease associated lung disease, ankylosing spondylitis (AS) and AS-associated lung disease, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, psoriasis type 1, psoriasis type 2, plaque psoriasis, moderate to severe chronic plaque psoriasis, autoimmune neutropaenia, sperm autoimmunity, multiple sclerosis (all subtypes), acute rheumatic fever, rheumatoid spondylitis, Sjogren's syndrome, autoimmune thrombocytopaenia.

In certain embodiments, the compounds of the invention are useful in the treatment of chronic kidney disease that have an inflammatory component, such as systemic lupus erythematosus (SLE) and lupus nephritis.

A. Rheumatoid Arthritis (RA)

In certain embodiments, the compounds of the invention can be used to treat Rheumatoid Arthritis (RA), including reducing signs and symptoms, inducing major clinical response, inhibiting the progression of structural damage, and improving physical function in adult patients, such as adult patients with moderately to severely active RA.

The compounds of the invention may be used alone, or in combination with methotrexate or other non-biologic disease-modifying anti-rheumatic drugs (DMARDs), and/or in combination with anti-TNFα biological agents, such as TNF antagonists like chimeric, humanized or human TNF antibodies, adalimumab (such as HUMIRA™ brand adalimumab), infliximab such as CA2 (REMICADE™ brand infliximab), golimumab such as SIMPONI™ (golimumab), certolizumab pegol such as CIMZIA™, tocilizumab such as ACTEMRA™, CDP 571, and soluble p55 or p75 TNF receptors, derivatives, thereof, etanercept such as p75TNFR1gG (ENBREL™ brand etanercept) or p55TNFR1gG (lenercept) (see above).

Patients having active rheumatoid arthritis (RA) may be diagnosed according to 1987-revised American College of Rheumatology (ACR) classification criteria or the 2010 ACR/EULAR criteria. In certain embodiments, RA may be diagnosed based on patients having at least 6 swollen and 9 tender joints. In certain embodiments, patients treatable with the subject compounds may include those who have failed therapy with at least one (e.g., at least one but no more than four) DMARDs and/or have inadequate response to methotrexate, adalimumab, infliximab, etanercept, or other anti-TNFα biological agents, or non-anti-TNF biologics.

In certain embodiments, the compound of the invention halts disease progression, and/or relieves at least a symptom of the disease, which may be detected or monitored by X-ray results, including radiographic progression of joint damage.

In certain embodiments, therapeutic efficacy can be measured by improvements in ACR20, ACR50, and/or ACR70, either in individual patients or a population of patients in need of treatment. In certain embodiments, statistically significant improvement (as compared placebo or untreated control) over a treatment period (e.g., 1 week, 2 weeks, 4 weeks, 2 months, 3 months, 6 months, 1 year, 2 years, 5 years, 10 years or more) in one or more of the ACR criteria is achieved. Statistical significance is manifested by a p value of less than 0.05, or less than 0.01.

Components of the ACR responses are well known in the art, and may include the median number of tender joints, the median number of swollen joints, physician global assessment such as one measured by visual analog scale, patient global assessment such as one measured by visual analog scale, pain such as one measured by visual analog scale, disability index of the Health Assessment Questionnaire (HAQ score), and CRP (mg/dL).

In certain embodiments, a major clinical response, defined as maintenance of an ACR70 response over a 6-month period, is achieved.

In certain embodiments, structural joint damage can be assessed radiographically and expressed as change in Total Sharp Score (TSS) and its components, the erosion score and Joint Space Narrowing (JSN) score, for example, at month 12 compared to baseline.

In certain embodiments, improvement in signs and symptoms of the disease can be measured by patient physical function response, such as disability index of Health Assessment Questionnaire (HAQ-DI), and/or the health-outcomes as assessed by The Short Form Health Survey (SF 36). Improvement can also be measured by one or both of Physical Component Summary (PCS) and the Mental Component Summary (MCS). Improvements can further be measured by Work Instability Scale for RA (RA-WIS) (see Gilworth et al., Arthritis & Rheumatism (Arthritis Care & Research) 49(3): 349-354, 2003, incorporated by reference).

B. Juvenile Idiopathic Arthritis (JIA)

In certain embodiments, the compounds of the invention can be used to treat Juvenile Idiopathic Arthritis (JIA), including reducing signs and symptoms of moderately to severely active polyarticular JIA in pediatric patients, such as those 4 years of age and older. In certain embodiments, the JIA patients show signs of active moderate or severe disease despite previous treatment with NSAIDs, analgesics, corticosteroids, or DMARDS.

In certain embodiments, signs and symptoms of the JIA and improvement thereof is measured by Pediatric ACR30, ACR50, and/or ACR70 over a treatment period.

C. Psoriatic Arthritis (PsA)

In certain embodiments, the compounds of the invention can be used to treat Psoriatic Arthritis (PsA), including reducing signs and symptoms, inhibiting the progression of structural damage, and improving physical function in adult patients with active PsA. In certain embodiments, treatable patients include those with moderately to severely active PsA (e.g., those with >3 swollen and >3 tender joints). In certain embodiments, such patients have had an inadequate response to NSAID therapy, which may be in one of the following forms: (1) distal interphalangeal (DIP) involvement; (2) polyarticular arthritis (absence of rheumatoid nodules and presence of plaque psoriasis); (3) arthritis mutilans; (4) asymmetric PsA; or (5) AS-like.

In certain embodiments, the compounds of the invention halts disease progression, and/or relieves at least a symptom of the disease, which may be measured by improvements in one or more measures of disease activity. In certain embodiments, measures of disease activity may include ACR20, ACR50, and/or ACR70, either in individual patients or a population of patients in need of treatment. In certain embodiments, statistically significant improvement (as compared placebo or untreated control) over a treatment period (e.g., 1 week, 2 weeks, 4 weeks, 2 months, 3 months, 6 months, 1 year, 2 years, 5 years, 10 years or more) in one or more of the ACR criteria is achieved. Statistical significance is manifested by a p value of less than 0.05, or less than 0.01.

Components of the ACR responses are well known in the art, and may include the median number of tender joints, the median number of swollen joints, physician global assessment such as one measured by visual analog scale, patient global assessment such as one measured by visual analog scale, pain such as one measured by visual analog scale, disability index of the Health Assessment Questionnaire (HAQ score), and CRP (mg/dL).

In certain embodiments, patients with psoriatic involvement of at least three percent body surface area (BSA) can also be evaluated for Psoriatic Area and Severity Index (PASI) responses in order to assess the extent of disease progression, and/or relieving of at least a symptom of the disease.

In certain embodiments, the compounds of the invention halts disease progression, and/or relieves at least a symptom of the disease, which may be measured by radiographic changes of hands, wrists, and feet. Changes in Total Sharp Score (TSS) and its components, the erosion score and Joint Space Narrowing (JSN) score, for example, may be measured at a predetermined time, such as 1, 2, 3, 6, or 12 months compared to baseline. A modified Total Sharp Score (mTSS), which included distal interphalangeal joints (i.e., not identical to the TSS used for rheumatoid arthritis), may be used by readers blinded to treatment group to assess the radiographs.

In certain embodiments, improvement in signs and symptoms of the disease can be measured by patient physical function response, such as disability index of Health Assessment Questionnaire (HAQ-DI), and/or the health-outcomes as assessed by The Short Form Health Survey (SF 36). Improvement can also be measured by one or both of Physical Component Summary (PCS) and the Mental Component Summary (MCS).

D. Ankylosing Spondylitis (AS)

In certain embodiments, the compounds of the invention can be used to treat Ankylosing Spondylitis (AS), including reducing signs and symptoms in adult patients with active AS. Active AS may be defined as patients who fulfills at least two of the following three criteria: (1) a Bath AS disease activity index (BASDAI) score ≧4 cm, (2) a visual analog score (VAS) for total back pain ≧40 mm, and (3) morning stiffness ≧1 hour. In certain embodiments, the patients may have inadequate response to glucocorticoids, NSAIDs, analgesics, methotrexate or sulfasalazine.

In certain embodiments, the compounds of the invention halts disease progression, and/or relieves at least a symptom of the disease, which may be measured by improvements in one or more measures of disease activity. In certain embodiments, measures of disease activity may include ASAS20/50/70. In certain embodiments, improvements in one or more measures of disease activity leads to a low level of disease activity (defined as a value <20 [on a scale of 0 to 100 mm] in each of the four ASAS response parameters) after a specific treatment period. The four ASAS response parameters include: ASAS20 Response Criteria; BASDAI (Bath Ankylosing Spondylitis Disease Activity Index) Score; BASMI (Bath Ankylosing Spondylitis Metrology Index) Score; and CRP level (mg/dL). The ASAS20 Response Criteria itself includes Patient's Global Assessment of Disease Activity (% of subjects with at least a 20% and 10-unit improvement measured on a Visual Analog Scale (VAS) with 0=“none” and 100=“severe”); Total Back Pain; Inflammation (mean of Questions 5 and 6 of BASDAI); and BASFI (Bath Ankylosing Spondylitis Functional Index).

In certain embodiments, improvement in signs and symptoms of the disease can be measured by patient physical function response, such as Ankylosing Spondylitis Quality of Life Questionnaire (ASQoL) score, and the Short Form Health Survey (SF-36) Physical Component Summary (PCS) score.

E. Crohn's Disease (CD)

In certain embodiments, the compounds of the invention can be used to treat Crohn's Disease (CD), including reducing signs and symptoms and inducing and maintaining clinical remission in adult patients with moderately to severely active Crohn's disease who have had an inadequate response to conventional therapy, reducing signs and symptoms and inducing clinical remission in these patients if they have also lost response to or are intolerant to one or more anti-TNFα biological agents, such as infliximab, adalimumab, and/or etanercept.

In certain embodiments, the treatable adult patients include those with moderately to severely active Crohn's disease, defined as having Crohn's Disease Activity Index (CDAI)≧220 and ≦450).

In certain embodiments, therapeutic efficacy of the subject compounds can be measured by induction of clinical remission (defined as CDAI<150). In certain embodiments, therapeutic efficacy of the subject compounds can be measured by induction of clinical response (defined as reduction of CDAI of at least 70 points). In certain embodiments, therapeutic efficacy of the subject compounds can be measured by maintenance of clinical remission over a pre-determined period, such as 6 months or 1 year under a treatment program or regimen.

In certain embodiments, therapeutic efficacy of the subject compounds can also be measured according to Example 11.

F. Ulcerative Colitis (UC)

In certain embodiments, the compounds of the invention can be used to treat Ulcerative Colitis (UC), including inducing and sustaining clinical remission in adult patients with moderately to severely active ulcerative colitis who have had an inadequate response to immunosuppressants such as corticosteroids, azathioprine or 6-mercaptopurine (6-MP), and reducing signs and symptoms in patients who have lost response to or were intolerant to TNF blockers, such as infliximab, adalimumab, and/or etanercept.

In certain embodiments, moderately to severely active ulcerative colitis is defined as having Mayo score 6 to 12 on a 12 point scale, with an endoscopy subscore of 2 to 3 on a scale of 0 to 3. In certain embodiments, the patient has concurrent or prior treatment with immunosuppressants such as corticosteroids, azathioprine, or 6-MP.

In certain embodiments, therapeutic efficacy of the subject compounds can be measured by induction of clinical remission (defined as Mayo score ≦2 with no individual subscores >1) at a specified treatment period, such as at week 1, 2, 4, 8, 12, or 16 after the commencement of treatment.

G. Plaque Psoriasis (Ps)

In certain embodiments, the compounds of the invention can be used to treat Plaque Psoriasis (Ps), including the treatment of adult patients with moderate to severe chronic plaque psoriasis who are candidates for systemic therapy or phototherapy, and when other systemic therapies are medically less appropriate.

In certain embodiments, moderate to severe chronic plaque psoriasis (Ps) patients are those who are candidates for systemic therapy or phototherapy; have chronic Ps with ≧10% body surface area (BSA) involvement, have Physician's Global Assessment (PGA) of at least moderate disease severity, and have Psoriasis Area and Severity Index (PASI)≧12 within three treatment periods.

In certain embodiments, therapeutic efficacy of the subject compounds can be measured by the proportion of patients who achieved “clear” or “minimal” disease on the 6-point PGA scale and the proportion of patients who achieved a reduction in PASI score of at least 75% (PASI 75) from baseline at a pre-determined treatment point (e.g., Week 4, 8, 12, 16, 20, or 24).

In certain embodiments, therapeutic efficacy of the subject compounds can be measured by the proportion of subjects who maintained a PGA of “clear” (defined as having no plaque elevation, no scale, plus or minus hyperpigmentation or diffuse pink or red coloration) or “minimal” (e.g., possible but difficult to ascertain whether there is slight elevation of plaque above normal skin, plus or minus surface dryness with some white coloration, plus or minus up to red coloration) disease or a PASI 75 response by a pre-determined treatment time point (e.g., after Week 33 and on or before Week 52).

### 4. Combination Therapy

Compounds of the invention can be used alone or in combination with one or more additional agent(s), e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition, e.g., an agent that affects the viscosity of the composition.

The compounds of the invention can be administered prior to, subsequent to or simultaneously with the additional pharmaceutical agent, whichever course of administration is appropriate.

In certain embodiments, the compounds of the invention and the additional pharmaceutical agents act either additively or synergistically.

For example, compounds of the invention may be administered in a pharmaceutically acceptable form either alone or in combination with one or more additional agents which modulate a mammalian immune system or with anti-inflammatory agents. These agents may include but are not limited to cyclosporin A (e.g., SANDIMMUNE® or NEORAL®, rapamycin, FK-506 (tacrolimus), leflunomide, deoxyspergualin, mycophenolate (e.g., CELLCEPT®), azathioprine (e.g., IMURAN®), daclizumab (e.g., ZENAPAX®), OKT3 (e.g., ORTHOCLONE®), AtGam, aspirin, acetaminophen, aminosalicylate, ciprofloxacin, corticosteroid, metronidazole, probiotic, tacrolimus, ibuprofen, naproxen, piroxicam, and anti-inflammatory steroids (e.g., prednisolone or dexamethasone). In certain embodiments, the one or more additional agents is selected from the group consisting of: aspirin, acetaminophen, aminosalicylate, ciprofloxacin, corticosteroid, cyclosporine, metronidazole, probiotic, tacrolimus, ibuprofen, naproxen, piroxicam, prednisolone, dexamethasone, anti-inflammatory steroid, methotrexate, chloroquine, azathioprine, hydroxychloroquine, penicillamine, sulfasalazine, leflunomide, tocilzumab, anakinra, abatacept, certolizumab pegol, golimumab, vedolizumab, natalizumab, ustekinumab, rituximab, efalizumab, belimumab, etanercept, infliximab, adalimumab, or an immune modulator (e.g., activator) for CD4⁺CD25⁺ T_{reg} cells.

These agents may be administered as part of the same or separate dosage forms, via the same or different routes of administration, and on the same or different administration schedules according to standard pharmaceutical practice.

For example, FK506 (Tacrolimus) is given orally at 0.10-0.15 mg/kg body weight, every 12 hours, within first 48 hours postoperative. Does is monitored by serum Tacrolimus trough levels. Cyclosporin A (SANDIMMUNE® oral or intravenous formulation, or NEORAL®, oral solution or capsules) is given orally at 5 mg/kg body weight, every 12 hours within 48 hours postoperative. Dose is monitored by blood Cyclosporin A trough levels.

It should further be understood that the combinations which are to be included within this invention are those combinations useful for their intended purpose. The agents set forth below are illustrative for purposes and not intended to be limiting. The combinations, which are part of this invention, can be the compounds of the present invention and at least one additional agent selected from the lists below. The combination can also include more than one additional agent, e.g., two or three additional agents if the combination is such that the formed composition can perform its intended function.

In certain embodiments, combinations are with non-steroidal anti-inflammatory drug(s) also referred to as NSAIDS, which include drugs like ibuprofen. Other combinations are corticosteroids including prednisolone; the well known side-effects of steroid use can be reduced or even eliminated by tapering the steroid dose required when treating patients in combination with the compounds of this invention.

Non-limiting examples of therapeutic agents for rheumatoid arthritis with which a compound of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. Compounds of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L). Combinations of therapeutic agents may interfere at different points in the autoimmune and subsequent inflammatory cascade; such examples may include TNF antagonists like chimeric, humanized or human TNF antibodies, adalimumab (such as HUMIRA™ brand adalimumab), infliximab such as CA2 (REMICADE™ brand infliximab), golimumab such as SIMPONI™ (golimumab), certolizumab pegol such as CIMZIA™, tocilizumab such as ACTEMRA™, CDP 571, and soluble p55 or p75 TNF receptors, derivatives, thereof, etanercept such as p75TNFR1gG (ENBREL™ brand etanercept) or p55TNFR1gG (lenercept), and also TNFα converting enzyme (TACE) inhibitors; similarly IL-1 inhibitors (Interleukin-1-converting enzyme inhibitors, IL-1RA etc.) may be effective for the same reason. Other combinations include Interleukin 11. Yet other combinations are the other key players of the autoimmune response which may act parallel to, dependent on or in concert with IL-18 function; especially IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another combination is non-depleting anti-CD4 inhibitors. Yet other combinations include antagonists of the co-stimulatory pathway CD80 (B7.1) or CD86 (B7.2) including antibodies, soluble receptors or antagonistic ligands.

A compound of the invention may also be combined with nonbiologic DMARDS or other agents, such as methotrexate, 6-mercaptopurine, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., NIK, IKK, p38 or MAP kinase inhibitors), IL-1β converting enzyme inhibitors, T-cell signalling inhibitors such as kinase inhibitors, metalloproteinase inhibitors, sulfasalazine, 6-mercaptopurines, angiotensin converting enzyme inhibitors, soluble cytokine receptors and derivatives thereof (e.g., soluble p55 or p75 TNF receptors and the derivatives p75TNFRIgG (Enbrel™ brand etanercept) and p55TNFRIgG (lenercept), sIL-1RI, sIL-1RII, sIL-6R), anti-inflammatory cytokines (e.g., IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, S1P1 agonists (such as FTY720), PKC family inhibitors (such as Ruboxistaurin or AEB-071) and Mesopram. In certain embodiments, combinations include methotrexate or leflunomide, and in moderate to severe RA cases, cyclosporine and anti-TNFα antibodies as noted above.

Non-limiting examples of therapeutic agents for inflammatory bowel disease (IBD) with which a compound of the invention can be combined may include (but are not limited to) the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, IL-23, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFα or IL-1 (e.g., NIK, IKK, or MAP kinase inhibitors); IL-1β converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and anti-inflammatory cytokines (e.g., IL-4, IL-10, IL-11, IL-13 and TGFβ).

Examples of therapeutic agents for Crohn's disease with which a compound of the invention can be combined include the following: TNF antagonists, for example, anti-TNF antibodies, adalimumab (such as HUMIRA™ brand adalimumab), infliximab such as CA2 (REMICADE™ brand infliximab), CDP 571, TNFR-Ig constructs, etanercept such as p75TNFRIgG (ENBREL™ brand etanercept) and lenercept such as p55TNFRIgG (LENERCEPT™) inhibitors and PDE4 inhibitors.

A compound of the invention can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors; 6-mercaptopurine; IL-11; mesalamine; prednisone; azathioprine; mercaptopurine; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis (MS) with which a compound of the invention can be combined include the following: corticosteroids; prednisolone; methylprednisolone; azathioprine; cyclophosphamide; cyclosporine; methotrexate; 4-aminopyridine; tizanidine; interferon-β1a (AVONEX®; Biogen); interferon-β1b (BETASERON®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE®; Teva Pharmaceutical Industries, Inc.); hyperbaric oxygen; intravenous immunoglobulin; cladribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of the invention can be combined with antibodies to cell surface molecules such as CD2, CD3, CD4, CD8, CD19, CD20, CD25, CD28, CD30, CD40, CD45, CD69, CD80, CD86, CD90 or their ligands. A compound of the invention may also be combined with agents such as methotrexate, cyclosporine, FK506, rapamycin, mycophenolate mofetil, leflunomide, an S1P1 agonist, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors, sIL-1RI, sIL-1RII, sIL-6R) and anti-inflammatory cytokines (e.g. IL-4, IL-10, IL-13 and TGFβ). Examples of therapeutic agents for multiple sclerosis in which a compound of the invention can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of the invention may also be combined with agents, such as alemtuzumab, dronabinol, daclizumab, mitoxantrone, xaliproden hydrochloride, fampridine, glatiramer acetate, natalizumab, sinnabidol, α-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for ankylosing spondylitis (AS) with which a compound of the invention can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, and anti-TNF antibodies, adalimumab (such as HUMIRA™ brand adalimumab), infliximab such as CA2 (REMICADE™ brand infliximab), CDP 571, TNFR-Ig constructs, etanercept such as p75TNFRIgG (ENBREL™ brand etanercept) and lenercept such as p55TNFRIgG (LENERCEPT™).

Non-limiting examples of therapeutic agents for psoriasis (Ps, such as moderate to severe plaque psoriasis) with which a compound of the invention can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine, ABT-874, ustekinamab, and adalimumab (such as HUMIRA™ brand adalimumab).

Non-limiting examples of therapeutic agents for psoriatic arthritis (PsA) with which a compound of the invention can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, adalimumab (such as HUMIRA™ brand adalimumab), and efalizumab.

Examples of therapeutic agents for SLE (Lupus) with which a compound of the invention can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of the invention may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of the invention may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of the invention can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of the invention may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, adalimumab (such as HUMIRA™ brand adalimumab), infliximab such as CA2 (REMICADE™ brand infliximab), CDP 571, TNFR-Ig constructs, etanercept such as p75TNFRIgG (ENBREL™ brand etanercept) and lenercept such as p55TNFRIgG (LENERCEPT™).

In certain embodiments, a compound of the invention is not used in combination with biologic DMARDs, such as TNFα antagonists described hereinabove, or in combination with potent immunosuppressants, such as azathioprine and cyclosporine.

A compound of the invention may also be combined with an immune modulator for CD4⁺CD25⁺ T_{reg} cells. T_{reg} cells are essential for maintaining normal immune homeostasis. In patients with autoimmune diseases, reduced numbers or functional impairment of T_{reg} cells has been observed, leading to loss of this finely-tuned mechanism. A humanized agonistic monoclonal antibody, BT-061, binds to a unique epitope of human CD4, and induces T_{reg}-specific signaling events that lead to their functional activation. Pre-clinical data using isolated T_{reg} cells and RA synovial fluid indicate that BT-061 leads to suppression of CD4⁺ and CD8⁺ T effector cell proliferation, reduction of the expression of pro-inflammatory cytokines, and increase in the production of the anti-inflammatory cytokine TGFβ. Thus similar immune modulators for CD4⁺CD25⁺ T_{reg} cells can also be co-administered with a compound of the invention for treating any of the inflammatory disease/disorder, or an autoimmune disease/disorder described herein, including but not limited to Rheumatoid Arthritis (RA), Crohn's disease, ankylosing spondylitis (AS), psoriatic arthritis, psoriasis, ulcerative colitis, systemic lupus erythematosus (SLE), lupus nephritis, diabetic nephropathy, dry eye syndrome, Sjogren's Syndrome, alopecia areata, vitiligo, or atopic dermatitis. In certain embodiments, the combination treats RA, CD, psoriasis, or psoriatic arthritis, including moderately to severely active RA, CD, psoriasis, or psoriatic arthritis. In certain embodiments, the RA, CD, psoriasis, or psoriatic arthritis patient being treated has inadequately responded to or has discontinued therapy due to loss of response to or intolerance to a first line therapy (such as a DMARD, including methotrexate) or an anti-TNFα therapy.

In certain embodiments, the immune modulator has one or more (or all) of the following properties: (1) activates a subset of CD4⁺ T cells comprising CD4⁺CD25⁺ regulatory T cells (T_{reg}), or CD4⁺CD25⁺ T_{reg} cells; (2) binds only to a special epitope of the human CD4 antigen (such as the IgG-like C2 type 1 domain of CD4), which said epitope of human CD4 may be bound by a mouse IgG1 anti-CD4 monoclonal antibody B-F5 or a humanized version thereof, such as the BT-061 hB-F5 antibody tregalizumab as described in U.S. Pat. No. 7,452,981 (incorporated herein by reference, including all sequences of the V_{H} and V_{L} chains disclosed therein); (3) provides an activation signal to naturally occurring T_{reg} cells but does not activate conventional T cells (e.g., CD4⁺ T cells that are not activated in (1), CD8⁺ cytotoxic T cells, etc.); and (4) is not a depleting anti-CD4 antibody that depletes CD4⁺ T cells, and/or does not appreciably trigger ADCC or CDC.

Representative such immune modulators are described in U.S. Pat. No. 7,452,981. In certain embodiments, the immune modulator is a humanized hB-F5 antibody derived from mouse monoclonal anti-CD4 antibody B-F5, wherein the hB-F5 antibody comprises V domains selected from the group consisting of: a) H chain V domain comprising the sequence, EEQLVESGGGLVKPGGSLRLSCAASGFSFSDCRMY WLRQAPGKGLEWIGVISVKSENYGANYAESVRGRFTJSRDDSKNTVYLQMNSLKT EDTAVYYCSA SYYRYDVGAWFAYWGQGTLVTVSS (SEQ ID NO: 1); b) L chain V domain comprising the sequence: DIVMTQSPDSLAVSLGERATFNCRASKSVSTSGYS YIYWYQQKPGQPPKLLIYLASILESGVPDRFSGSGSGTDFTLTISSLQAEDVAVYYC QHSRELPWTFGQGTKVEIK (SEQ ID NO: 2); and c) combinations thereof, wherein the hB-F5 antibody is able to activate a subset of T CD4⁺ cells comprising CD4⁺CD25⁺ cells.

In certain embodiments, the immune modulator comprises a fragment of the hB-F5 antibody above, wherein the fragment comprises the V domains of SEQ ID NO: 1 and SEQ ID NO: 2. In certain embodiments, the immune modulator comprises a polypeptide encoded by a polynucleotide selected from the group consisting of: a) a polynucleotide comprising a sequence encoding the H chain V domain of SEQ ID NO: 1; and b) a polynucleotide comprising a sequence encoding the L chain V domain of SEQ ID NO: 2. For example, the polynucleotide may be selected from the group consisting of: a) a polynucleotide comprising the sequence SEQ ID NO: 3 of U.S. Pat. No. 7,452,981 (the V domain of H chain of humanized antibody hBF-5, incorporated herein by reference); and b) a polynucleotide comprising the sequence SEQ ID NO: 4 of U.S. Pat. No. 7,452,981 (the V domain of K chain of humanized antibody hBF-5, incorporated herein by reference).

While not wishing to be bound by any particular theory, BT-061 (tregalizumab) is a humanized monoclonal antibody that activates CD4⁺CD25⁺ T-regulatory cells, which in turn suppresses effector T cells (such as CD4⁺ and/or CD8⁺ effector T cells) and strengthens a natural function of the body that prevents excessive immune reactions. Unlike other anti-CD4 antibodies that have been in development, BT-061 (tregalizumab) does not cause depletion of CD4 positive T-cells that would give rise to weakened immune responses.

In certain embodiments, the immune modulator (such as BT-061) is administered subcutaneously or via i.v. In certain embodiments, the immune modulator (such as BT-061) is administered once a week, s.c., at a dose of about 1.25 mg to 100 mg (such as 25 mg, 50 mg, or 75 mg per dose).

### 5. Administration and Dosage Forms

One or more compounds of this invention can be administered to a human patient by themselves or in pharmaceutical compositions where they are mixed with biologically suitable and pharmaceutically acceptable carriers or excipient(s), at doses to treat or ameliorate a disease or condition as described herein. Mixtures of these compounds can also be administered to the patient as a simple mixture or in suitable separately formulated pharmaceutical compositions.

In general, pharmaceutical compositions comprising the compounds of the invention may be manufactured in a manner that is itself known, e.g., by means of conventional mixing, dissolving, granulating, dragee-making, levigating, emulsifying, encapsulating, entrapping or lyophilizing processes. The techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral (e.g., selective release in certain parts of the small intestine), ophthalmic, eyedrop, rectal, transmucosal, topical, dermal, intra-luminal (e.g., via enema for GI or colon indications), or intestinal administration; parenteral delivery, including intramuscular, subcutaneous, intramedullary injections, as well as intrathecal, direct intraventricular, intravenous, intraperitoneal, intranasal, or intraocular injections.

Pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For example, a therapeutically effective amount of the active compounds of the invention may be formulated for oral, buccal, intranasal, parenteral (e.g., intravenous, intramuscular or subcutaneous) or rectal administration, or in a form suitable for administration by inhalation or insufflation. The active compounds of the invention may also be formulated for sustained delivery according to methods well known to those of ordinary skill in the art. Examples of such formulations can be found in U.S. Pat. Nos. 3,538,214, 4,060,598, 4,173,626, 3,119,742, and 3,492,397.

In certain embodiments, the compound of the invention in a pharmaceutical composition is administered to a subject orally, in a dosage form (e.g., a tablet or a capsule) formulated for oral administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration (see below).

For oral administration, the compounds can be formulated readily by combining the active compounds with pharmaceutically acceptable carriers and/or excipients well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

Examples of other carriers or excipients include but are not limited to calcium carbonate, calcium phosphate, various sugars, starches, cellulose derivatives, gelatin, and polymers such as polyethylene glycols.

In certain embodiments, a compound of the invention is formulated for oral administration as a capsule, in excipients that comprise microcrystalline cellulose, dibasic calcium phosphate, magnesium stearate, croscarmellose sodium, and hydroxypropyl cellulose.

In certain embodiments, the capsule or tablet is formulated as immediate and/or sustained release formulations.

If desired, disintegrating agents may be added, such as the cross-linked polyvinyl pyrrolidone, agar, or alginic acid or a salt thereof such as sodium alginate.

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the following Examples.

### a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

### b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

### c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In certain embodiments, the subject pharmaceutical formulation is formulated for oral administration, and for selective release of the active ingredient (e.g., Compound 1) in certain parts of the small intestine. For example, Blanchette et al. (Mat. Res. Soc. Syrup. Proc., 724:215-220, 2002, incorporated herein) reported the use of certain carrier materials for targeted delivery of a chemotherapeutic agent, bleomycin, to the upper small intestine in response to the pH shift when the formulation enters the upper small intestine from the stomach. Specifically, hydrogel nanospheres composed of methacrylic acid (MAA) and poly(ethylene glycol) (PEG) were loaded with bleomycin by in situ polymerization. Results showed that bleomycin release from the nanospheres was responsive to the pH of the environment surrounding the nanospheres.

Since the intraluminal pH is 4 to 5 in the duodenum but becomes progressively more alkaline, approaching 8 in the lower ileum, control of drug release based on specific pH values may facilitate the release of the drug at specific portions of the GI tract having the matching pH.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

For buccal administration, the compositions may take the form of tablets or lozenges formulated in conventional manner.

The compounds of the invention may be formulated for parenteral administration by injection (e.g., bolus injection or continuous infusion), including using conventional catheterization techniques or infusion. The compounds of the invention may be formulated in aqueous solutions, preferably in physiologically compatible buffers such as Hanks' solution, Ringer's solution, or physiological saline buffer. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

The compounds of the invention may also be formulated in rectal compositions such as suppositories or retention enemas, e.g., containing conventional suppository bases such as cocoa butter or other glycerides. Such compositions may be administered intraluminally, for example, for treatment of GI or colon indications, such as Crohn's disease, IBD, ulcerative colitis, etc.

Alternatively, the active ingredient may be in powder form for constitution with a suitable vehicle, e.g., sterile pyrogen-free water, before use.

For transmucosal administration, penetrants appropriate to the barrier to be permeated are used in the formulation. Such penetrants are known in the art.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5 W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few hours up to over several days. Depending on the chemical nature and the biological stability of the therapeutic reagent, additional strategies for protein stabilization may be employed.

### 6. Therapeutically Effective Amount

A “therapeutically effective amount” is an amount of a compound of the invention, or in certain embodiments a combination of two or more such compounds, which inhibits, totally or at least partially, the progression of the condition to be treated; or alleviates, at least partially, one or more symptoms of the condition to be treated. In certain embodiments, a therapeutically effective amount is efficacious in inhibiting one or more symptoms of the condition to be treated, yet does not lead to a significant undesirable side effect, such as negatively affecting erythropoiesis (e.g., as reflected by reduction of reticulocyte count) and/or NK cell function (e.g., as reflected in reduction of NK cell count).

In certain embodiments, a therapeutically effective amount can also be an amount which is prophylactically effective. In certain embodiments, however, therapeutically effective amount does not refer to an amount which is prophylactically effective. The amount which is therapeutically effective may depend upon the patient's size and gender, the condition to be treated, the severity of the condition and the result sought.

For a given patient, a therapeutically effective amount may be partly determined by methods known to those of skill in the art in view of the teaching herein.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity, such as JAK1 activity).

In some embodiments, it may be appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

Toxicity and therapeutic efficacy of the compounds of the invention can be determined by standard pharmaceutical procedures in cell cultures and/or experimental animals, e.g., for determining the maximum tolerated dose (MTD) and the ED₅₀ (effective dose for 50% maximal response). The dose ratio between toxic and therapeutic effects is the therapeutic index, and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage may be varied by the individual physician in view of the patient's condition (see, e.g., Fingl et al., 1975, in The Pharmacological Basis of Therapeutics, Ch. 1 p. 1), based on the dosage level described herein. In the treatment of crises, the administration of an acute bolus or an infusion approaching the MTD may be required to obtain a rapid response.

Dosage amount and interval may also be adjusted, e.g., individually, to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of protein kinase using the assays described herein. Dosages necessary to achieve the MEC may depend on individual characteristics and route of administration. However, HPLC assays or bioassays can be used to determine plasma concentrations.

Dosage intervals can also be determined using the MEC value. Compounds may be administered using a regimen which maintains plasma levels above the MEC for 10-90% of the time, preferably between 30-90% and most preferably between 50-90% until the desired amelioration of symptoms is achieved.

Alternatively or in addition, compounds may be administered using a regimen which achieves and maintains a desired AUC₀₋₂₄ value, such as an AUC₀₋₂₄ of about 0.05-1.50 μg·hr/mL, about 0.10-1.06 μg·hr/mL, about 0.10-0.50 μg·hr/mL, about 0.11-0.30 μg·hr/mL, about 0.12-0.15 μg·hr/mL, about 0.128 μg·hr/mL, and/or until the desired amelioration of symptoms is achieved.

As used herein, AUC₀₋₂₄ refers to “Area Under the concentration-time Curve from time zero to 24 hour post-dose.” It is calculated by first plotting the log 10 of cumulative plasma compound (e.g., Compound 1) concentration over a 24-hour period post-dose (e.g., an oral dose), and then calculating the area under the concentration-time curve so generated over the same time period.

In certain embodiments, the regimen is qd (once daily). In certain embodiments, the regimen is bid (twice daily), e.g., in equal amounts. In certain embodiments, the regimen is tid (thrice daily), e.g., in equal amounts. In certain embodiments, the regimen is qid (four times a day), e.g., in equal amounts.

In certain embodiments, a compound of the invention (e.g., the free base form of Compound 1), in the amount of about 2, 2.5, or 3 mg per unit dosage form (e.g., per capsule), is administered orally bid (twice daily) in equal amount (e.g., twice a day, about 2.5 mg each time) to a human patient.

In cases of local administration or selective uptake, however, the effective local concentration of the drug may not be related to plasma concentration. In that case, dosage may also be measured based on local AUC generated by local drug concentration.

In certain embodiments, the amount of composition administered may also be partly dependent on the subject being treated, e.g., on the subject's weight, the severity of the affliction, the manner of administration and the judgment of the prescribing physician.

### 7. Assays and Animal Models

The assays and animal models described herein are exemplary embodiments that may be used in the methods of the invention, such as assessing the extent of Jak kinase inhibition. The specific conditions set forth in the assays and animal models are intended to provide illustrative guidance, and are thus not limiting. One of ordinary skill in the art can readily alter the specific conditions or parameters illustrated without depart from the general teaching herein.

Human T-Blasts IL-2 pSTAT5 Cellular Assay

Interleukin-2 (IL-2) is believed to signal via a receptor complex that requires the kinase activity of both Jak1 and Jak3. To evaluate the effects of a compound of the invention (e.g., Compound 1) on Jak1/Jak3 inhibition in a cellular context, inhibition of IL-2-induced phosphorylation of STAT5 in human T-blasts by the compound of the invention (e.g., Compound 1) can be determined by measuring ex vivo stimulated IL-7-dependent STAT5 phosphorylation, which may be assessed by, for example, AlphaScreen SureFire readout (STAT5 p-Tyr694/699).

For example, T blast cells isolated from blood sample from a subject (e.g., a sample obtained before and/or after a dose of the subject pharmaceutical composition) can be subject to the ex vivo stimulated IL-7-dependent STAT5 phosphorylation assay to determine Jak1 kinase activity before and after treatment. Change in Jak1 activity can be used to determine the extent of Jak1 inhibition (e.g., [activity after−activity before]/(activity before)).

An illustrative ex vivo stimulated IL-7-dependent STAT5 phosphorylation assay is described below using commercially available materials. The same assay can be used for patient sample with minor modification.

### Materials:

Phytohemaglutinin T-blasts can be prepared from commercial sources, such as Leukopacks purchased from Biological Specialty Corporation, Colmar, Pa. 18915. The T-blasts can be cryopreserved in 5% DMSO/media prior to use in the assay.

For this assay the cells are thawed in assay medium typically having the following composition: RPMI 1640 medium (Gibco 11875093) with 2 mM L-glutamine (Gibco 25030-081), 10 mM HEPES (Gibco 15630-080), 100 μg/mL Pen/Strep (Gibco 15140-122), and 10% heat inactivated FBS (Gibco 10438026). Other materials used in the assay may include: DMSO (Sigma D2650), 96-well dilution plates (polypropylene) (Corning 3365), 96-well assay plates (white, ½ area, 96 well) (Corning 3642), D-PBS (Gibco 14040133), IL-2 (R&D 202-IL-10 (10 μg)), Alphascreen pSTAT5 kit (Perkin Elmer TGRS5S10K) and Alphascreen protein A kit (Perkin Elmer 6760617M).

### Methods:

T-Blasts are thawed and cultured for about 24 h without IL-2 prior to assay. Test compounds or controls are dissolved and serially diluted in 100% DMSO. DMSO stocks are subsequently diluted 1:50 in cell culture media to create the 4× compound stocks (containing 2% DMSO). Using a Corning white 96 well, ½ area plate, cells are plated at 2×10⁵/10 μl/well in 10 μL media followed by addition of 5 μL of 4× test compound in duplicate. Cells are incubated with compound for about 0.5 hr at about 37° C. Next, 5 μL of IL-2 stock is added at 20 ng/mL final concentration. IL-2 is stored as a 4 μg/mL stock solution, as specified by the manufacturer, at about −20° C. in aliquots and diluted 1:50 with assay media (to 80 ng/mL) just prior to use. The contents of the wells are mixed by carefully tapping sides of plate(s) several times followed by incubation at about 37° C. for about 15 min. The assay is terminated by adding 5 μL of 5× AlphaScreen lysis buffer and shaking on an orbital shaker for about 10 min at room temperature. Alphascreen acceptor bead mix is reconstituted following Perkin Elmer's protocol. 30 μL/well of reconstituted Alphascreen acceptor bead mix is added, covered with foil then shaken on orbital shaker for about 2 min on high then about 2 h on low. Donor bead mix is reconstituted following Perkin Elmer's AlphaScreen protocol; 12 μL/well are added, covered with foil then shaken for about 2 min on high, and about 2 h on low. Plates are read on an EnVision reader following Perkin Elmer's AlphaScreen protocol instructions.

TF-1 IL-6 pSTAT3 Cellular Assay

Interleukin-6 (IL-6) is believed to signal via a receptor complex that requires the kinase activity of two Jak1 molecules. To evaluate the effects of the subject compound (e.g., Compound 1) on Jak1 inhibition in a cellular context, inhibition of IL-6-induced phosphorylation of STAT3 in human erythroblasts, such as erythroleukemia TF-1 cells (ATCC CRL-2003), can be assessed by IL-6 pSTAT3 cellular assay using, for example, AlphaScreen SureFire readout (STAT3 p-Tyr705). Human erythroblasts may be isolated from patient sample using any art recognized methods, such as Hu et al. (“Isolation and functional characterization of human erythroblasts at distinct stages: implications for understanding of normal and disordered erythropoiesis in vivo,” Blood, 121(16):3246-3253, 2013, incorporated by reference).

### Materials:

TF-1 cells (ATCC #CRL-2003). Culture medium: DMEM medium (Gibco 11960-044) with 2 mM L-glutamine (Gibco 25030-081), 10 mM HEPES (Gibco 15630-080), 100 μg/mL Pen/Strep (Gibco 15140-122), 1.5 g/L sodium bicarbonate (Gibco 25080-094), 1 mM sodium pyruvate (Gibco 11360-070), 10% heat inactivated FBS (Gibco 10437-028), and 2 ng/mL GM-CSF (R&D 215-GM-010). Other materials used in this assay may include: DMSO (Sigma D2650), 96-well dilution plates (polypropylene) (Corning 3365), 96-well assay plates (white, ½ area, 96 well) (Corning 3642), D-PBS (Gibco 14040133), IL-6 (R&D 206-IL/CF-050 (50 μg)), Alphascreen pSTAT3 kit (Perkin Elmer TGRS3S10K) and Alphascreen protein A kit (Perkin Elmer 6760617M).

### Methods:

Prior to the assay, cells are cultured for about 18 hr in the culture medium without GM-CSF. Test compounds or controls are dissolved and serially diluted in 100% DMSO. DMSO stocks are subsequently diluted 1:50 in cell culture media to create the 4× compound stocks (containing 2% DMSO). Using a Corning white 96 well, ½ area plate, cells are plated at 2×10⁷/10 μL/well in 10 μL media followed by addition of 5 μL of the 4× test compound stock in duplicate. Cells are incubated with compound for about 0.5 hr at about 37° C. followed by addition of 5 μL of 400 ng/mL IL-6. IL-6 is stored in 10 μg/mL aliquots using endotoxin free D-PBS (0.1% BSA) at about −20° C. Prior to assay IL-6 is diluted to 400 ng/mL in culture media and applied (5 μL/well) to all wells, except to negative control wells where 5 μL/well of media is added. The contents of the wells are mixed carefully by tapping the side of the plate several times. Plates are incubated at about 37° C. for about 30 min. Cells are lysed by adding 5 μL of 5× AlphaScreen cell lysis buffer to all wells, shaken for about 10 min at room temperature then assayed. Alternatively, assay plates may be frozen at about −80° C. and thawed later at room temperature. Using the pSTAT3 SureFire Assay kit (Perkin Elmer #TGRS3S10K) acceptor bead mix is reconstituted following Perkin Elmer's AlphaScreen protocol instructions. 30 μL are added per well then the plate is covered with foil and shaken on an orbital shaker for about 2 min on high, then about 2 h on low at RT. Donor bead mix is reconstituted following Perkin Elmer's AlphaScreen protocol instructions. 12 μL are added per well, then covered with foil and shaken on orbital shaker for about 2 min on high, then about 2 h on low at about 37° C. Plates are read on an EnVision reader following Perkin Elmer's AlphaScreen protocol instructions at RT.

UT7/EPO pSTAT5 Cellular Assay

UT-7 is a cell line established from the bone marrow of a patient with acute megakaryoblastic leukemia. The growth of UT-7 cells strictly depends on GM-CSF (1 ng/mL), IL3 (10 units/mL) or EPO (1 unit/mL). The proliferation of UT-7 cells is also stimulated by IL6. UT-7/EPO is a subline of UT-7 cells established from UT-7 cells maintained for more than 6 months in the presence of EPO. The growth of UT-7/EPO is not supported by GM-CSF or IL3. This cell line can also be used to assess the ability of a subject compound to inhibit Jak1 kinase activity induced by EPO stimulation.

Alternatively or in addition, an ex vivo assay dependent on GM-CSF driven STAT phosphorylation may be similarly conducted using patient sample (such as patient blood sample). In fact, all the Jak inhibitor compounds tested so far are consistently more potent against GM-CSF signaling than EPO signaling by the same degree. Hence the GM-CSF stimulated STAT phosphorylation assay may be used as a surrogate for inhibition potency measure.

### Materials:

UT7/EPO cells are passaged with erythropoietin (EPO), split twice per week and fresh culture medium is thawed and added at time of split. Culture Medium: DMEM medium (Gibco 11960-044) with 2 mM L-glutamine (Gibco 25030-081), 10 mM HEPES (Gibco 15630-080), 100 U/mL Pen/Strep (Gibco 15140-122), 10% heat inactivated FBS (Gibco 10437-028), EPO (5 μL/mL=7.1 μL of a 7 μg/mL stock per mL of medium). Assay media: DMEM, 2 mM L-glutamine, 5% FBS, 10 mM HEPES. Other materials used in the assay may include: DMSO (Sigma D2650), 96-well dilution plates (polypropylene) (Corning 3365), 96-well assay plates (white, ½ area, 96 well) (Corning 3642), D-PBS (Gibco 14040133), IL-2 (R&D 202-IL-10 (10 μg)), Alphascreen pSTAT5 kit (Perkin Elmer TGRS5S10K) and Alphascreen protein A kit (Perkin Elmer 6760617M).

### Methods:

Culture cells for about 16 hr without EPO prior to running assay. Test compounds or controls are dissolved and serially diluted in 100% DMSO. DMSO stocks are subsequently diluted 1:50 in cell culture media to create the 4× compound stocks (containing 2% DMSO). Using a Corning white 96 well, ½ area plate, cells are plated at 2×10⁵/10 μL/well in 10 μL media followed by addition of 5 μL of 4× test compound stock in duplicate. Cells are incubated with compound for about 0.5 hr at about 37° C. After incubation, 5 μL of EPO is added to afford a final concentration of 1 nM EPO. The contents of the wells are mixed by carefully tapping sides of the plate several times followed by incubation at about 37° C. for about 20 min. 5 μL of 5× AlphaScreen lysis buffer are added followed by shaking on an orbital shaker for about 10 min at RT. 30 μL/well of acceptor beads are added after reconstitution following Perkin Elmer's AlphaScreen protocol, covered with foil and shaken on orbital shaker for about 2 min on high, then about 2 h on low. Donor beads are reconstituted following Perkin Elmer's AlphaScreen protocol instructions followed by addition of 12 μL/well, covered with foil and shaken on an orbital shaker for about 2 min on high, about 2 h on low. Plates are read on an EnVision reader following Perkin Elmer's AlphaScreen protocol instructions.

### Acute In Vivo Measurement of JAK Inhibition by Compounds is Measured Using the: Concanavalin A (Con A)-Induced Cytokine Production in Lewis Rats

The test compound is formulated in an inert vehicle (for example but not limited to 0.5% hydroxypropylmethyl cellulose (Sigma, cat #H3785)/0.02% Tween 80 (Sigma, cat #4780) in water) at the desired concentration to achieve doses in the range of 0.01-100 mg/kg. Six-week-old male Lewis rats (125 g-150 g) (Charles River Laboratories) are dosed with the compound orally, at time zero (0 min). After about 30 min the rats are injected intravenously (i.v.) with 10 mg/kg Concanavalin A (Con A, AmershamBioscience, cat #17-0450-01) dissolved in PBS (Invitrogen, cat #14190). About 4 h later, the rats are cardiac bled and their plasma is analyzed for levels of IL-2 (ELISA kit: R&D Systems cat #R2000) and IFN-γ (ELISA kit: R&D Systems cat #RIF00).

### Chronic In Vivo Effects of the Compounds on an Arthritis Disease Model is Measured Using: Adjuvant Induced Arthritis (AIA) in a Lewis Rat

Female Lewis rats, (6 weeks of age, 125 g-150 g in weight from Charles River Laboratories) are immunized intradermally (i.d.) in the right hind-footpad with 100 μL of a suspension of mineral oil (Sigma, cat # M5905) and containing 200 μg M. tuberculosis, H37RA (Difco, cat #231141). The inflammation appears in the contra-lateral (left) hind paw seven days after the initial immunization. Seven days post immunization, the compound is formulated in an inert vehicle (for example but not limited to 0.5% hydroxypropylmethyl cellulose (Sigma, cat #H3785)/0.02% Tween 80 (Sigma, cat #4780) in water) and dosed orally once or twice a day for at least 10 days. Baseline paw volume is taken on day 0 using a water displacement pleythsmograph (Ugo Basile North America Inc. PA 19473, Model #7140). Rats are lightly anesthetized with an inhalant anesthetic (isoflurane) and the contra-lateral (left) hind paw is dipped into the plethysmograph and the paw volume is recorded. The rats are scored every other day up to day 17 after immunization. On day 17 after immunization, all rats are exsanguinated by cardiac puncture under isoflurane anesthesia, and the left hind paw is collected to assess the impact on bone erosion using micro-CT scans (SCANCO Medical, Southeastern, Pa., Model # μCT 40) at a voxel size of 18 μm, a threshold of 400, sigma-gauss 0.8, support-gauss 1.0. Bone volume and density is determined for a 360 μm (200 slice) vertical section encompassing the tarsal section of the paw. The 360 μm section is analyzed from the base of the metatarsals to the top of the tibia, with the lower reference point fixed at the tibiotalar junction. Drug exposure is determined in the plasma using LC/MS.

### Collagen Induced Arthritis (CIA) in a Lewis Rat

On day −1 Collagen Type II (CII), soluble from bovine nasal septum (Elastin Products, Cat #CN276) is weighed out for a dose of 600 μg/rat, 0.01M acetic acid (150 μL HOAc USP grade. J. T. Baker, order#9522-03, and 250 mL Milli Q Water) is added for a concentration of 4 mg/mL. The vial is covered with aluminum foil and placed on a rocker at about 4° C. overnight. On day 0 collagen stock solution is diluted 1:1 with Incomplete Freunds adjuvant (IFA) (Difco labs, cat #263910) using a glass Hamilton luer lock syringe (SGE Syringe Perfection VWR cat #007230), final concentration 2 mg/mL. Female Lewis rats (Charles River Laboratories) acclimated for 7 days at the time of immunization weighing approximately 150 g are anesthetized in an anesthesia chamber using isoflurane (5%) and oxygen. Once the rats are completely anesthetized, they are transferred to a nose cone to maintain anesthesia during the injections. Rats are shaved at the base of the tail, 300 μL of collagen is injected i.d. on the rump of the rat, n=9 per group. 100 μL at three sites with a 500 μL leur lock syringe and a 27 g needle. IFA control rats are injected in the same manner (n=6). The IFA is a 1:1 emulsion with the 0.01M acetic acid. Boost is done on day 6 of the study. Shaving is not done on this day and injections are done in the same manner as the immunization. The inflammation appears in both hind paws 10 days after the initial immunization. 10 days post immunization, the compound is formulated in an inert vehicle (for example but not limited to 0.5% hydroxypropylmethyl cellulose (Sigma, cat #H3785)/0.02% Tween 80 (Sigma, cat #4780) in water) and dosed orally once or twice a day for at least 9 days. Baseline paw volume was taken on day 7 using a water displacement pleythsmograph (Vgo Basile North America Inc. PA 19473, Model #7140). Rats are lightly anesthetized with an inhalant anesthetic (isoflurane) and both hind paws are dipped into the plethysmograph and the paw volume is recorded. The rats are scored 2 to 3 times a week up to day 18 after immunization. On day 18 after immunization, all rats are exsanguinated by cardiac puncture under isoflurane anesthesia, and the hind paws are collected to assess the impact on bone erosion using micro-CT scans (SCANCO Medical, Southeastern, Pa., Model # μCT 40) at a voxel size of 18 μm, a threshold of 400, sigma-gauss 0.8, support-gauss 1.0. Bone volume and density is determined for a 360 μm (200 slice) vertical section encompassing the tarsal section of the paw. The 360 μm section is analyzed from the base of the metatarsals to the top of the tibia, with the lower reference point fixed at the tibiotalar junction. Drug exposure is determined from plasma using LC/MS.

### Chronic In Vivo Effects of the Compounds on an Asthma Disease Model is Measured Using: OVA Induced Rat Asthma Model

Female Brown Norway rats (7-9 weeks of age) are sensitized on day 0 and 7 with 40 μg ovalbumin (OVA) (Sigma-Aldrich, St. Louis, Mo.) in a 20 mg/ml solution of Imject Alum (Pierce, Rockford, Ill.). The rats are subsequently challenged intratracheally on day 19 and 20 with 1.5 μg OVA in 50 μL PBS. Dosing of inhibitor began on day 18 and continues through day 22. On day 22, 48 h after the second challenge, rats are subjected to an anesthetized and restrained pulmonary function test. Airway hyper responsiveness (AHR) is assessed using whole body plethysmography. Briefly, a surgical plane of anesthesia is induced with an intraperitoneal injection of 60 mg/kg ketamine and 5 mg/kg xylazine (Henry Schein, Inc., Melville, N.Y.). A tracheal cannula is surgically inserted between the 3rd and 4th tracheal rings. Spontaneous breathing is prevented by jugular vein injection of 0.12 mg/kg pancuronium bromide (Sigma-Aldrich, St Louis, Mo.). Animals are placed in a whole body plethysmograph (Buxco Electronics, Inc., Wilmington, N.C.) and mechanically ventilated with 0.2 mL room air at 150 breaths per minute with a volume controlled ventilator (Harvard Apparatus, Framingham, Mass.). Pressure in the lung and flow within the plethysmograph are measured using transducers and lung resistance is calculated as pressure/flow using Biosystem Xa software (Buxco Electronics). Airway resistance is measured at baseline and following challenge with 3, 10, and 30 mg/mL methacholine (Sigma Aldrich, St. Louis, Mo.) delivered with an inline ultrasonic nebulizer. Upon completion of pulmonary function testing, the lungs were lavaged 3 times with 1 mL sterile PBS. The volume from the first wash is centrifuged at 2000 rpm for 5 min, and the supernatant is stored for subsequent analysis. The volume of washes 2 through 3 are added to the pellet derived from the first wash and subsequently processed for evaluation of cellular infiltrate by flow cytometry. Plasma is collected from blood drawn from the vena cava and is used for evaluation of drug concentrations.

Method for Measuring T, B, NK and NKT Cells in Blood Samples from Orally Dosed Human TBNK Cell Counts:

The following procedure is provided to illustrate a method of counting TBNK cells from a blood sample, such as one collected from a human subject orally dosed with a compound of the invention. The method (including the type of instruments and reagents used) as described herein in is non-limiting, as minor variations to the procedure and equivalent instruments and reagents can be readily envisioned.

Specifically, a blood sample is collected into 3 mL lavender K2 EDTA Vacutainer at specified time points and tubes mixed. Samples are shipped to analysis facility at ambient temperature overnight, and sample analysis is preferably done on the day received.

Two BD Trucount tubes (BD Biosciences, cat #340334) are used per sample and labeled with sample number followed by A or B. Two tubes are also set up for the BD Multi-Check Control (BD Biosciences, cat #340911) that is a stabilized blood sample used as an inter-run control each time samples are analyzed. Using BD MultiTest IMK kit (BD Biosciences, cat #340503), 20 μL of antibody mix A is placed into all A tubes and 20 μL of antibody mix B is placed into all B tubes as per manufacturer's instructions. Each blood sample (50 μL) is placed in both tube A and B as per manufacturer's instructions. Tubes are vortexed gently to mix and put into the dark for 15 minutes. 450 μL of 1× Fix/lysis buffer is added to all tubes and vortexed gently to mix. Tubes are incubated for 15 minutes in the dark and then run on a BD FACScalibur using BD Multitest software for analysis of cell counts (e.g., T cells, B cells, NK cells). BD Calibrite 3 Beads as well as APC beads are used for setup and calibration of machine (BD Biosciences, cat #340486 and 23056). The Multitest software is used to calculate cell numbers for T lymphocytes, B lymphocytes, and NK cells.

For NKT cell determination, cell numbers are determined by using FlowJo and looking at the CD3⁺/CD16⁺/CD56⁺ population. The number of cells/μL is calculated by using the following equation: (the number of events in cell population/the number of events in absolute bead count region)×(the number of beads per test*/test volume). *This value is found on BD Trucount tube label.

Method for Measuring Inhibition of IL-6 and Common Gamma Chain Signalling Ex Vivo in Blood Samples from Orally Dosed Human

### Ex Vivo Stimulation Assay:

The following procedure is provided to illustrate a method of measuring inhibition of IL-6 and common gamma chain signaling through the respective Jak kinases, from a blood sample, such as one collected from a human subject orally dosed with a compound of the invention. The method (including the type of instruments and reagents used) as described herein in is non-limiting, as minor variations to the procedure and equivalent instruments and reagents can be readily envisioned.

Specifically, for each subject, blood is collected by venipuncture into 2 mL sodium heparin tubes at time=0, 1 hr, 6 hr, 12 hr (or other pre-determined intervals). Blood is kept on ice and each time point is processed as soon after collection as possible. For each time point, 150 μL of blood is added to six 15 mL conical tubes and incubated for 10 minutes at 37° C. Recombinant human IL-6 (R&D Systems, cat #206-IL, 400 ng/mL), IL-7 (R&D Systems, cat #207-IL, 400 ng/mL), or GM-CSF (R&D Systems, cat #215-GM, 80 ng/mL) is added to one tube each and dPBS is added to three tubes (negative control for each cytokine) in a volume of 50 μL and mixed well. Tubes are incubated 10 minutes at 37° C. Tubes are then placed on ice and anti-CD14-APC antibody (BD Biosciences, cat #340436, 3 μL/tube) is added to the IL-6 and GM-CSF (−/+) tubes and mixed well. Anti-CD3-FITC antibody (BD Biosciences, cat #555339, 3 μL/tube) is added to the IL-7 tubes (−/+) and mixed well. These are incubated on ice for 20 minutes. Fix/Lyse buffer (BD Biosciences, cat #558049, 1.8 mL/tube of 1× pre-warmed to 37° C.) is added to all tubes and mixed vigorously and incubated for 10 minutes at 37° C. Samples are then washed 2× with Fix/lyse buffer and pellets brought up in 100 μL fix/lyse buffer and frozen at −70° C. or colder and shipped on dry ice.

Intra-cellular staining is done by thawing samples at room temperature, transferring to a 96-well round bottom plate (Costar #3799) and washing pellets 2× (centrifuge at 411×g for 5 minutes, then flick out supernatant) with dPBS/2% FBS before adding 200 μL of BD Perm buffer III (BD Biosciences, cat #558050, chilled to −20° C.) to pellets and incubating on ice, covered with foil for 30 minutes. Samples are again washed 2× with dPBS/2% FBS and blocked for 20′ on ice with dPBS/2% BSA with human IgG (Sigma, cat #14506-50, 1 μg/100 μL/sample). Samples are washed again 1× and pellets are brought back up in 100 μL of block solution with the appropriate BD phospho STAT-PE Antibody. Anti-pSTAT5-PE (BD Bioscience, cat #612567, 4 μL/well/100 μL) is added to IL-7 and GM-CSF samples and anti-pSTAT3-PE (BD Biosciences, cat #558557, 4 μL/well/100 μL) is added to the IL-6 samples. This is incubated for 60-90 minutes at room temperature, covered with foil and then run on the FACSCalibur HTS.

GeoMeans are determined using FlowJo analysis software as follows: for the IL-7 (−/+) samples by gating on CD3⁺ cells from the lymphocyte population, and for the IL-6 and GM-CSF (−/+) samples, gating on the CD14⁺ cells in the monocyte/macrophage population. Compensation is done for the IL-7 samples using FlowJo software after running BD CompBeads (BD Biosciences, cat #552843) with both the CD3-FITC Ab and PE-pSTAT5 Ab separately. Percent change in pSTAT levels is calculated as follows: % change=1−(Ave ΔGeomean Tn/Ave ΔGeomean T0), ΔGeomean=(+cytokine)−(no cytokine).

### Reticulocyte Deployment Assays

Reticulocytes were deployed with an i.v. EPO injection, which produced a modest but very precise reticulocytosis. Compound 1 or tofacitinib was dosed 30 minutes prior to EPO injection, and then once every 12 hours subsequently for three days.

### PK/PD Modeling

A direct maximum enhancement model was the most predictive for defining the efficacious concentration range and human efficacious dose. Efficacious AUC was based on paw swelling on the last day of the study plotted against the Log 10 of cumulative plasma concentration of Compound 1 or tofacitinib over 12 hours (AUC₀₋₁₂). In AIA rats, approximately 60% inhibition of paw swelling (AUC₆₀) is achieved by the 10 mg bid AUC exposure levels (˜300 ng*hr/mL). By comparison, the efficacious exposure (AUC₆₀) of Compound 1 was estimated to be 85 ng*hr/mL using this methodology.

### EXAMPLES

The ability of the compounds of the invention or their pharmaceutically acceptable salts to inhibit Janus Kinase 1 and, consequently, demonstrate their effectiveness for treating disorders or conditions characterized by Janus Kinase 1 is shown by the following examples.

It should be understood, however, that the following examples are for illustrative purposes and are not to be construed as limiting the scope of the present invention.

### Example 1

### Potency of Compound 1 Against Jak Kinases and Related Kinases

The potency of Compound 1 on recombinant Jak family kinase domains was determined using in vitro reactions using adenosine-5′-triphosphate (ATP) as a competitive inhibitor.

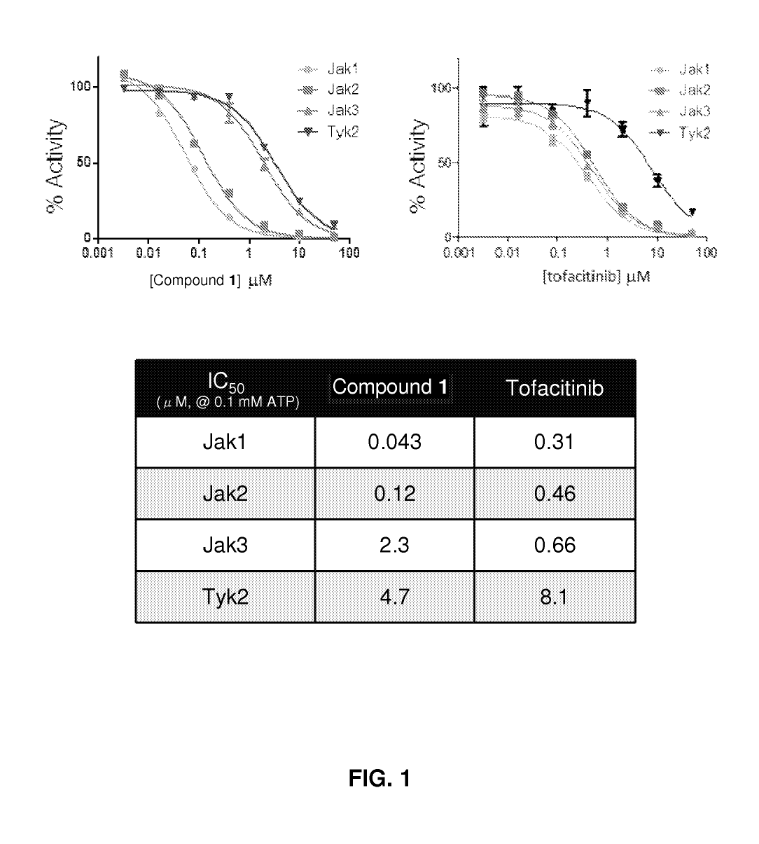

The results indicated that Compound 1 is an ATP competitive inhibitor, and is most potent against Jak1 with concentration at 50% inhibition (IC₅₀) of about 0.045 μM when tested at 0.1 mM ATP (FIG. 1) and <0.003 μM at 0.001 mM ATP (data not shown).

Compound 1 displays good selectivity in a panel of 60+ protein kinases that also includes Jak3 (data not shown). Of the kinases in the panel, 14 kinases have an Compound 1 IC₅₀ below 10 μM, but only 2 non-Jak kinases have IC₅₀'s below 1 μM (Rock1 at 0.55 μM and Rock2 at 0.43 μM).

### Example 2

### Potency of Compound 1 in Cellular and Other Assays

Interleukin-2 (IL-2) is believed to signal via a receptor complex that requires the kinase activity of both Jak1 and Jak3. To evaluate the effects of Compound 1 on Jak1/Jak3 inhibition in a cellular context, inhibition of IL-2-induced phosphorylation of STAT5 in human T-blasts by Compound 1 was assessed by AlphaScreen SureFire readout (STAT5 p-Tyr694/699). The IC₅₀ for Compound 1 was 21±4 nM (Table 1).

Interleukin-6 (IL-6) is believed to signal via a gp130/IL-6Rα receptor complex that requires the kinase activity of 2 Jak1 molecules. To evaluate the effects of Compound 1 on Jak1 inhibition in a cellular context, inhibition of IL-6-induced phosphorylation of STAT3 in human erythroleukemia TF-1 cells by Compound 1 was assessed by AlphaScreen SureFire readout (STAT3 p-Tyr705). The IC₅₀ for Compound 1 was 9±5 nM (Table 1).

Erythropoeitin (EPO) is believed to signal via a receptor complex that requires the kinase activity of 2 Jak2 molecules. To evaluate the effects of Compound 1 on Jak2 inhibition in a cellular context, inhibition of EPO-induced phosphorylation of STAT5 in the human EPO-dependent megakaryoblastic leukemic UT-7 cells was assessed by AlphaScreen SureFire readout (STAT5 p-Tyr694/699). The IC₅₀ for Compound 1 was 628±161 nM (Table 1).

The results of these assays consistently show the selectivity of Compound 1 for Jak1 over Jak2, and provide a basis for determining whether a more selective biochemical profile will translate into an improved clinical profile by sparing Jak2-dependent cellular pathway.

Similar assays were also conducted using the FDA approved drug for RA treatment, Tofacitinib, in place of Compound 1. Results from these experiments are also included in Table 1 for comparison.

TABLE 1
Potency of Compound 1 in Cellular Assays

| | Jak | IC₅₀ (nM) | | IC₅₀ (nM) |
|---|---|---|---|---|
| Assay | Involved | Compound 1 | | Tofacitinib |
| IL-6 pSTAT3 in TF-1 | Jak1 | 9 ± 5 | (n = 16) | 43 |
| cells | | | | |
| EPO pSTAT5 in UT-7 | Jak2 | 628 ± 161 | (n = 17) | 1,110 |
| cells | | | | |
| IL-2 pSTAT5 in T-blasts | Jak1, Jak3 | 21 ± 4 | (n = 5) | — |

Tofacitinib was added as a benchmark because of the availability of clinical data potentially correlating with cellular selectivity. As shown in Table 1 above and Table 1A below, Compound 1 was approximately 74-fold more selective for Jak1 (about 8-9 nM) over Jak2 (about 600 nM). Tofacitinib, by comparison, was about 24-fold more selective using the same assays (44 vs. 1110). Both compounds were somewhat more potent against Jak2 when EPO signaling was assessed in erythroid bone marrow colony forming assays (Table 1A). Paired measurements indicated a statistically significant increase in the selectivity ratio of Compound 1 compared to tofacitinib (p-value=0.0014).

The potency of Compound 1 and several related molecules to tofacitinib, against GM-CSF and IL-3 signaling. Like EPO, GM-CSF and IL-3 depend on Jak2 but use receptor complexes that are members of the common beta chain family. Surprisingly, signaling of these common beta chain cytokines was consistently less sensitive than EPO signaling to all compounds tested (data not shown). This result suggests that Jak2 sensitivity appears to be sensitive to receptor context.

Finally, compound potency in whole blood was measured against IL-6 signaling and against signaling by the common gamma chain cytokines IL-7 and IL-15 (Table 1A). Here, Compound 1 was consistently 4-5 fold more potent against IL-6 signaling than tofacitinib, whereas there was modest but statistically significant two fold difference in their potency against common gamma chain signaling (Table 1A). The values obtained for tofacitinib in these studies are very similar to those reported elsewhere (Meyer et al., J. Inflamm., (Lond.) 7:41, 2010).

TABLE 1A
Compound 1 and Tofacitinib potency in
cellular assays and bone marrow colony forming assays

| | Cellular Assays | | | Whole Blood Assays | | | Colony |
|---|---|---|---|---|---|---|---|
| | IC₅₀ (nM) | | | IC₅₀ (nM) | | | Formation |
| | IL-6 | EPO | Jak1/2 | IL-7 | IL-15 | IL-6 | EPO |
| | TF-1 Cells | UT-7 Cells | Potency | pSTAT5 | pSTAT5 | pSTAT3 | (Erythroid |
| Compound | pSTAT3 | pSTAT5 | Ratio | CD3+ | CD3+ | CD14+ | CFU) |
| Tofacitinib | 44 | 1110 | 24 | 31 | 22 | 114 | 0.8 |
| Comp. 1 | 8 | 608 | 74 | 14 | 16 | 17 | 0.4 |

It is apparent that Compound 1 is a more selective Jak1 inhibitor over Jak2 based on these assays. The Jak1/Jak2 potency ratio, defined as the inverse ratio of IC₅₀ values for Jak1 over Jak2, is about 74-fold for Compound 1 (e.g., about 1/(9/628)), and about 24-fold for Tofacitinib (e.g., about 1/(43/1110)).

In another set of experiments in which the Jak1/Jak3 potency ratio is measured, the potency ratio for Compound 1 is about 58-fold, while the potency ratio for Tofacitinib is about 2-fold. Thus experimental data demonstrates that Compound 1 is a much more selective Jak1 inhibitor (over both Jak2 and Jak3) as compared to the commercial drug Tofacitinib, which is less selective against Jak2 and marginally selective against Jak3. See Table 2 (Jak1 and Jak3 inhibition expressed as IC₅₀ value in μM).

TABLE 2
Comparing Potency Ratio of Jak1/Jak3

| | | | Jak1/Jak3 |
|---|---|---|---|
| | Jak1 | Jak3 | Potency Ratio |
| Tofacitinib | 0.31 | 0.66 | 2× |
| Compound 1 | 0.04 | 2.3 | 58× |

### Example 3

### In vivo Potency as Measured by Animal Models

Jak1 in vivo potency was measured in acute (concanavalin A induced interferon [IFN]γ) and chronic (adjuvant-induced arthritis) rat models.

The rat concanavalin A (Con A) model was selected as the acute screening model because it provides an assessment of oral bioavailability, as well as a rapid measurement of effects on Jak1-dependent mechanisms. Intravenous injection of Con A into Lewis rats results in T cell receptor ligation and activation, leading to IL-2 release and IFN-γ induction by an autocrine mechanism. IL-2 release is Jak-independent, whereas IFN-γ induction is blocked by Jak1 inhibitors.

Rats are dosed orally 30 minutes prior to intravenous Con A to coincide with the oral time to maximum observed plasma concentration (Tₘₐₓ) for the majority of these inhibitors. Plasma levels of IFN-γ and IL-2 are assessed 4 hours later in a terminal bleed.

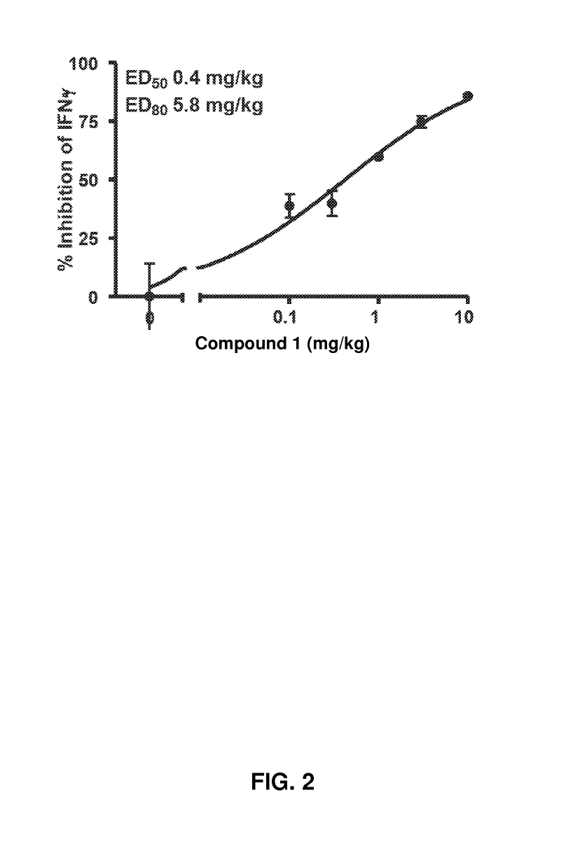

When Compound 1 was dosed orally as described above at 10, 3, 1.0, 0.3, and 0.1 mg/kg, a dose-dependent inhibition of IFN-γ release was observed, with an effect dose (ED)50 and ED80 of 0.4 mg/kg and 5.8 mg/kg respectively (FIG. 2).

The ability of Compound 1 to modulate inflammation in a complex disease setting was assessed using AIA (Adjuvant-Induced Arthritis model) in female Lewis rats, an established preclinical model for RA.

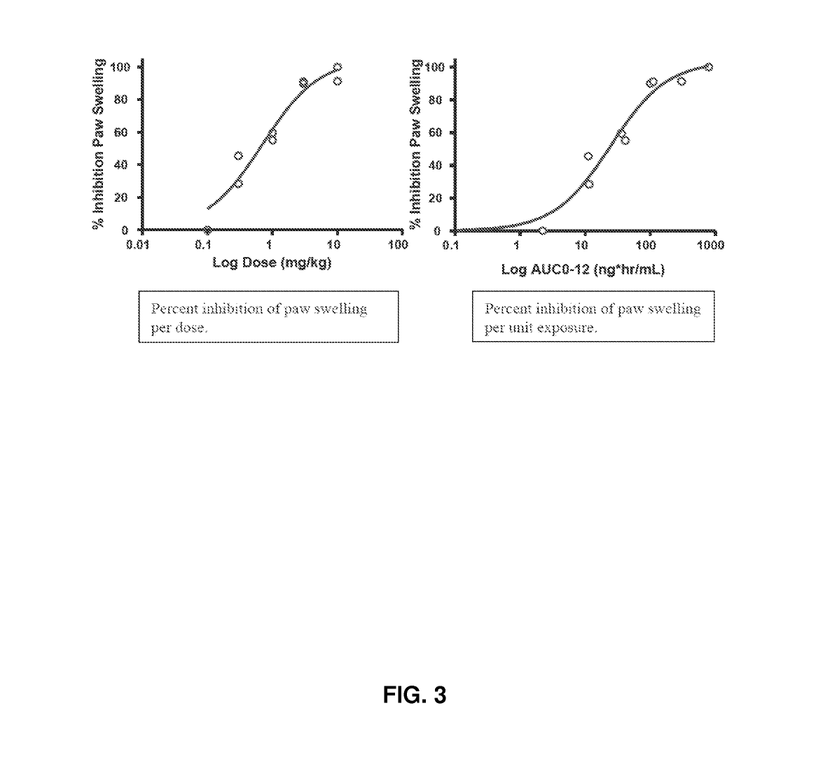

Oral doses of Compound 1 ranging from 0.1 to 10 mg/kg twice daily (bid) resulted in a dose and exposure-dependent reduction in paw swelling (FIG. 3, left). Efficacious AUC was the AUC resulting in 60% inhibition of the maximum response (AUC₆₀) based on paw swelling on the last day of the study plotted against the log 10 of cumulative plasma concentration of Compound 1 over 12 hours (AUC₀₋₁₂). The AUC₆₀ was calculated as total AUC₀₋₁₂ 85±20 ng·hr/mL and fraction unbound AUC₀₋₁₂ 34±7 ng·hr/mL (FIG. 3, right). In contrast, for the commercially available RA drug Tofacitinib administered at 10 mg bid, AUC₆₀ was calculated as total AUC₀₋₁₂ 392±121 ng·hr/mL and fraction unbound AUC₀₋₁₂ 300±98 ng·hr/mL (data not shown).

Data from the AIA experiments were used for the PK/pharmacodynamic (PD) modeling, as this is an established model for RA that mimics aspects of the complex disease setting. Retrospective modeling was performed using tofacitinib to establish a correlation between the preclinical and clinical data and to implement a translational PK/PD model. It is apparent from the modeling that a direct maximum enhancement model is the most predictive for defining the efficacious concentration range and human efficacious dose. Furthermore, utilizing the preclinical AUC60 in the AIA model resulted in a robust estimate for the efficacious exposures that have been observed in the clinic for tofacitinib.

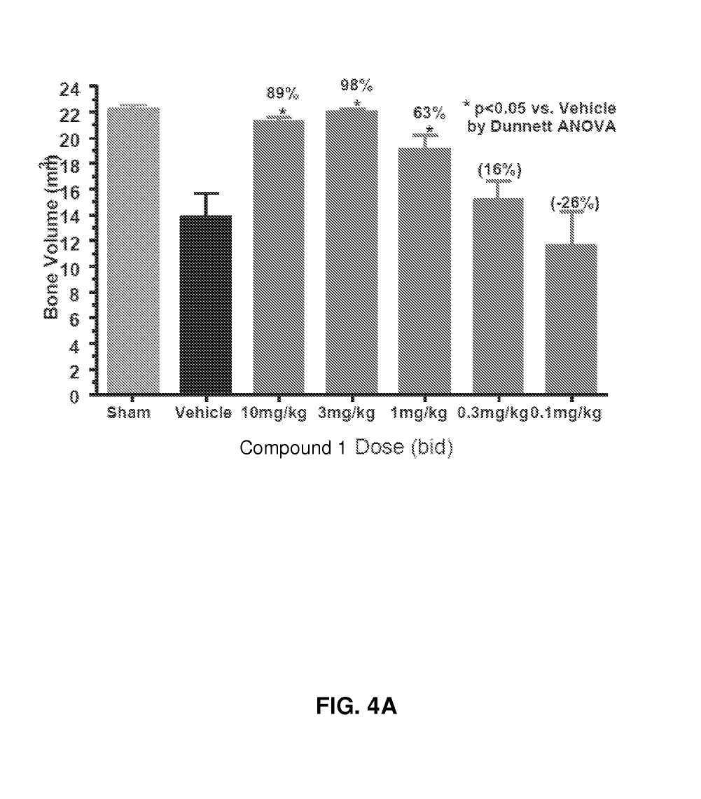

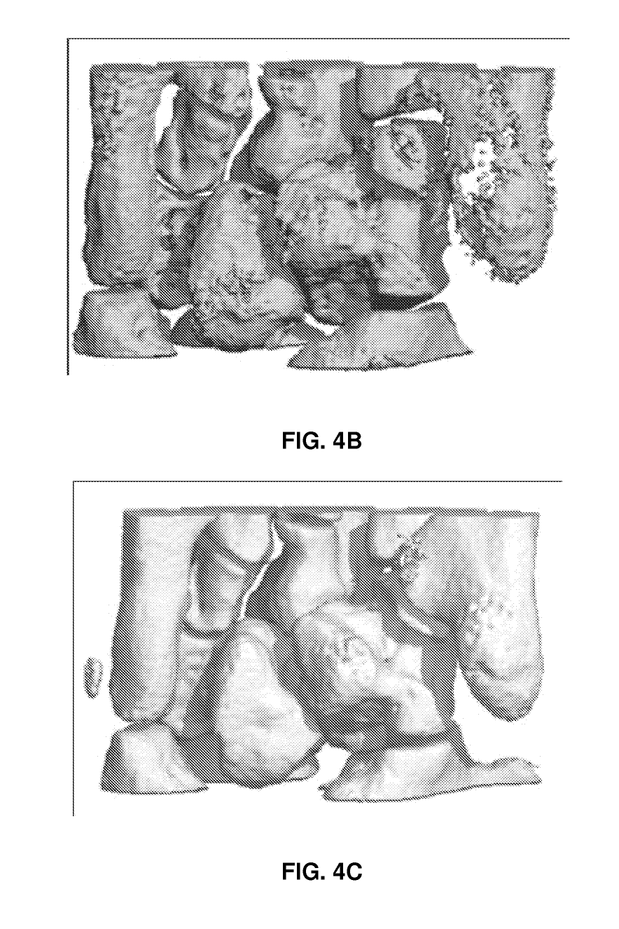

The efficacious exposure (AUC60) of Compound 1 was estimated to be 85 ng·hr/mL by this methodology (see above and FIG. 3). The equivalent of this efficacious exposure was used for safety margin calculations and human PK predictions. Furthermore, complete protection of bone erosion as assessed by measured change in tarsal bone volume by micro CT scanning was observed at 3 mg/kg bid (FIG. 4).

### Example 4

### Pharmacodynamics

The experiments aimed at measuring the level of Jak inhibition in vivo and in blood samples ex vivo are described herein to assess the degree of target (Jak1, Jak1/3) coverage in vivo per unit exposure and correlate that with efficacy.

Published results using the Jak inhibitor tofacitinib demonstrated effects on NK cells and CD8⁺ T cells in rodents, monkeys, and humans (Conklyn et al., J. Leukoc. Biol. 76(6):1248-1255, 2004; van Gurp et al., Transplantation 87(1):79-86, 2009). These observations are in line with tofacitinib potency against the T and NK cell growth/survival factors IL-2, IL-7, and IL-15.

Ex vivo Stimulation Assays

In the first experiment, orally dosed Compound 1 was shown to inhibit ex vivo stimulated IL-7-dependent STAT5 phosphorylation.

Unlike other common gamma chain receptors, IL-7 receptors are expressed relatively abundantly on peripheral blood T cells, enabling the use of IL-7 as an agonist in ex vivo stimulation assays. Stimulation of whole blood with IL-7 leads to phosphorylation of STAT5, which can be measured by flow cytometry. Because STAT proteins are Jak kinase substrates, inhibition of their phosphorylation can be considered a mechanistic readout for Jak inhibition.

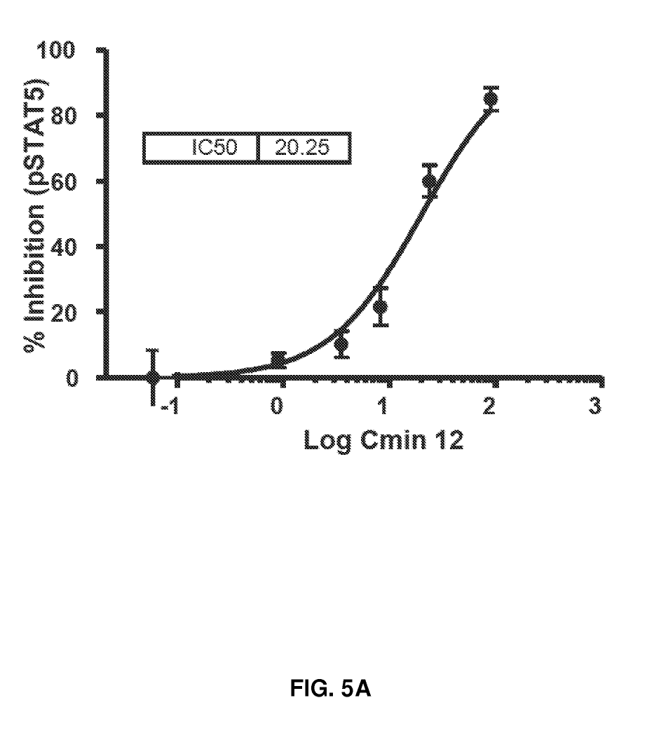

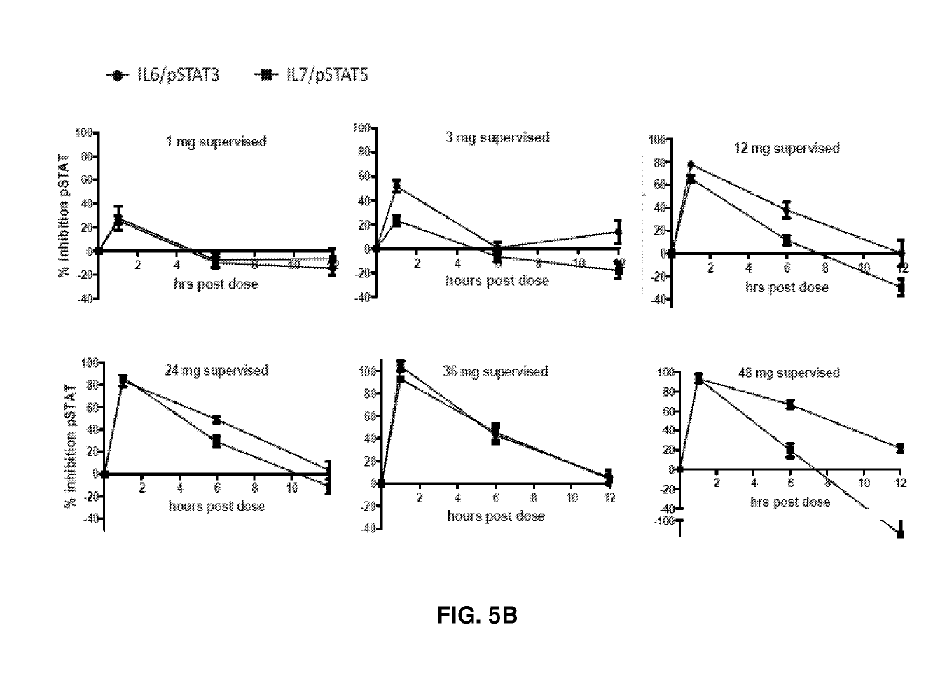

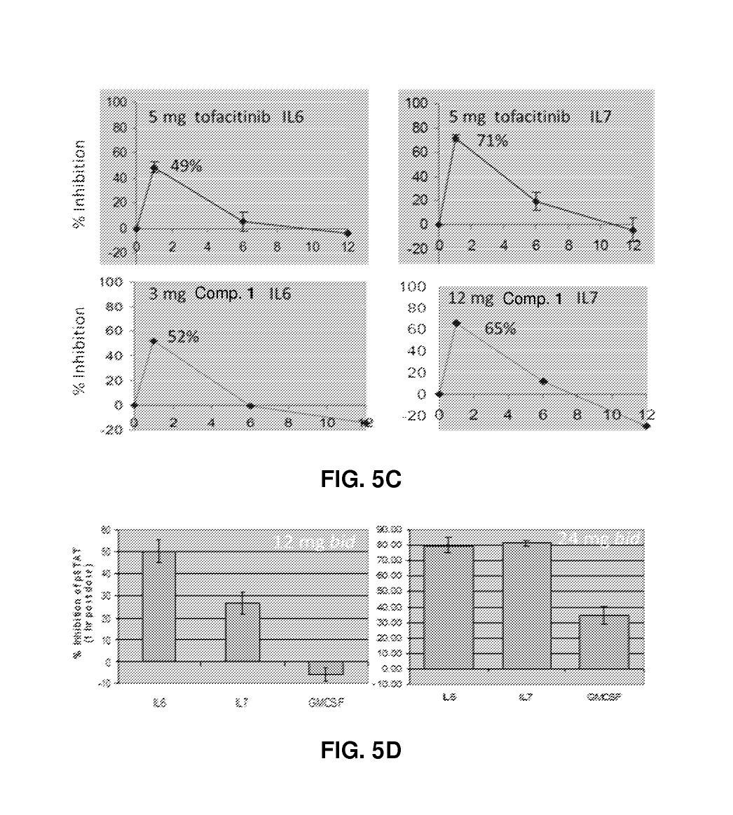

Here, rats were dosed bid with Compound 1 for 14 days, and blood was drawn at 12 hours after the final dose, then challenged with IL7. The exposure response relationship for Compound 1 inhibition of IL-7-induced pSTAT5 formation, yielding an IC₅₀ value of about 20 nM, is shown in FIG. 5A, which agrees well with the 11 nM IC₅₀ obtained in a similar rat whole blood assay.

A similar ex vivo stimulation experiment was conducted to measure inhibition of IL6 and common gamma chain signaling ex vivo in blood samples from orally dosed healthy human subjects. The results are shown in FIG. 5B.

Specifically, for the ex vivo stimulation assay, for each subject, blood was collected by venipuncture into 2 mL sodium heparin tubes at time=0, 1 hr, 6 hr, and 12 hr. Blood was kept on ice and each time point was processed as soon after collection as possible. For each time point, 150 μL of blood was added to six 15 mL conical tubes and incubated for 10 minutes at 37° C. Recombinant human IL-6 (R&D Systems, cat #206-IL, 400 ng/mL), IL-7 (R&D Systems, cat #207-IL, 400 ng/mL), or GM-CSF (R&D Systems, cat #215-GM, 80 ng/mL) was added to one tube each and dPBS was added to three tubes (negative control for each cytokine) in a volume of 50 μL and mixed well. Tubes were incubated 10 minutes at 37° C. Tubes were then placed on ice and anti-CD14-APC antibody (BD Biosciences, cat #340436, 3 μL/tube) was added to the IL-6 and GM-CSF (−/+) tubes and mixed well. Anti-CD3-FITC antibody (BD Biosciences, cat #555339, 3 μL/tube) was added to the IL-7 tubes (−/+) and mixed well. These were incubated on ice for 20 minutes. Fix/Lyse buffer (BD Biosciences, cat #558049, 1.8 mL/tube of 1× pre-warmed to 37° C.) was added to all tubes and mixed vigorously and incubated for 10 minutes at 37° C. Samples were then washed 2× with Fix/lyse buffer and pellets brought up in 100 μL fix/lyse buffer and frozen at −70° C. or colder and shipped on dry ice.

Intra-cellular staining was done by thawing samples at room temperature, transferring to a 96-well round bottom plate (Costar #3799) and washing pellets 2× (centrifuge at 411×g for 5 minutes, then flick out supernatant) with dPBS/2% FBS before adding 200 μL of BD Perm buffer III (BD Biosciences, cat #558050, chilled to −20° C.) to pellets and incubating on ice, covered with foil for 30 minutes. Samples were again washed 2× with dPBS/2% FBS and blocked for 20′ on ice with dPBS/2% BSA with human IgG (Sigma, cat #14506-50, 1 μL/100 μL/sample). Samples were washed again 1× and pellets were brought back up in 100 μL of block solution with the appropriate BD phospho STAT-PE Antibody. Anti-pSTAT5-PE (BD Bioscience, cat #612567, 4 μL/well/100 μL) was added to IL-7 and GM-CSF samples and anti-pSTAT3-PE (BD Biosciences, cat #558557, 4 μL/well/100 μL) was added to the IL-6 samples. This was incubated for 60-90 minutes at room temperature, covered with foil and then run on the FACSCalibur HTS.

GeoMeans were determined using FlowJo analysis software as follows: for the IL-7 (−/+) samples by gating on CD3⁺ cells from the lymphocyte population, and for the IL-6 and GM-CSF (−/+) samples, gating on the CD14⁺ cells in the monocyte/macrophage population. Compensation was done for the IL-7 samples using FlowJo software after running BD CompBeads (BD Biosciences, cat #552843) with both the CD3-FITC Ab and PE-pSTAT5 Ab separately. Percent change in pSTAT levels was calculated as follows: % change=1-(Ave ΔGeomean Tn/Ave ΔGeomean T0), ΔGeomean=(+cytokine)−(no cytokine).

FIG. 5C shows that single oral doses of 5 mg Tofacitinib and 3 mg Compound 1 have similar pharmacodynamic (PD) effects on Jak1 signaling in healthy human subjects, based on the ex vivo IL6-driven pSTAT3 assay plotted as % inhibition of IL6-driven STAT3 phosphorylation over the hours post administration (see above). The data shows that % inhibition of IL6-driven STAT3 phosphorylation by Tofacitinib and Compound 1 follows roughly the same PD profile, suggesting that at the respective doses tested, inhibition of Jak1 activity (and thus clinical efficacy) by Tofacitinib and Compound 1 are similar, if not identical. Moreover, 12 mg of Compound 1 was necessary to inhibit IL-7-induced pSTAT5 to a similar extent as that of 5 mg of tofacitinib (FIG. 5C), indicating that relative to tofacitinib, Compound 1 spares common gamma chain cytokine signaling.

In vivo Pharmacodynamics

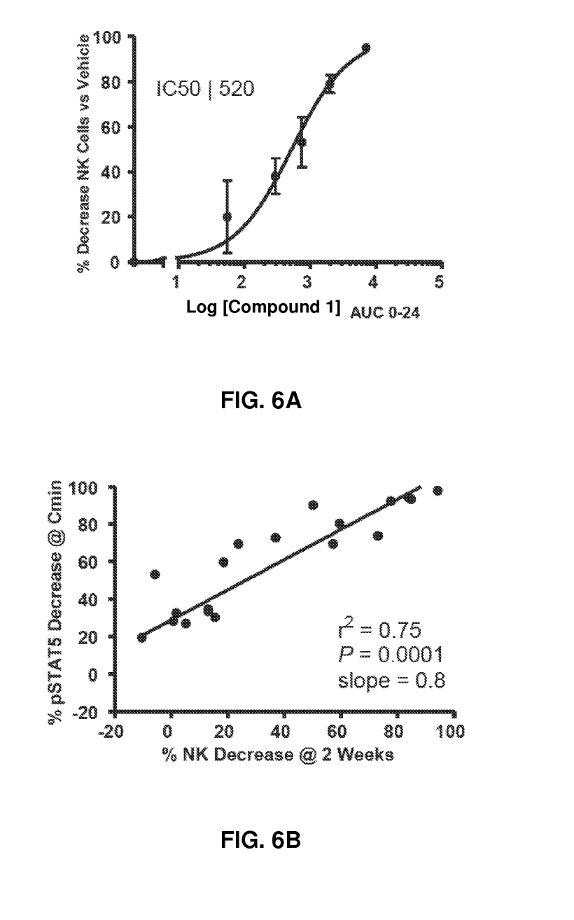

To measure pharmacodynamic effects, healthy rats were dosed bid with Compound 1 for 2 weeks, then assessed peripheral NK cell counts and inhibition of IL-7 induced pSTAT5 formation ex vivo. The exposure dependent effect of Compound 1 on peripheral NK cell counts is shown in the left panel of FIG. 6. The correlation of this effect with ex vivo cytokine signaling inhibition is shown in the right panel of FIG. 6. These measurements correlated well, confirming the Jak dependency of the NK cell count endpoint.

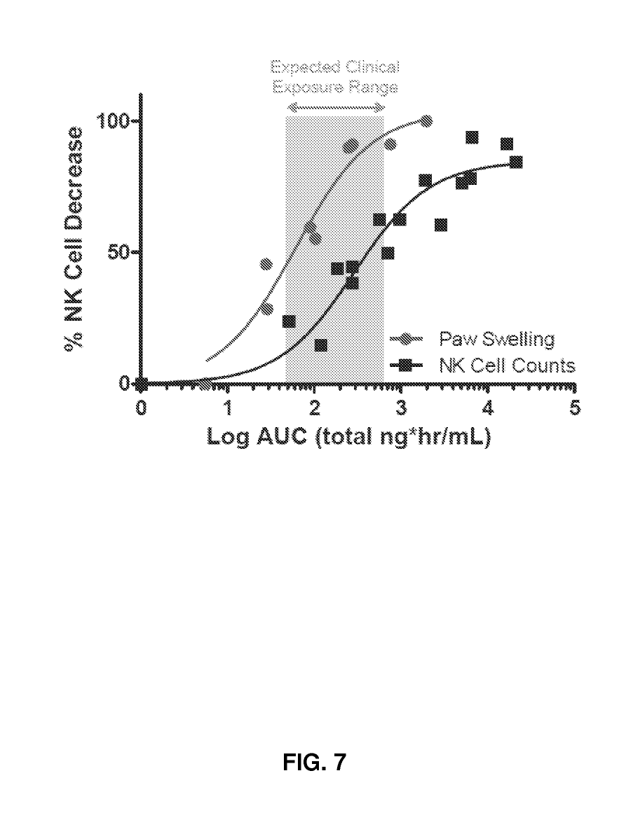

To establish the relationship between changes in NK cell counts and nonclinical efficacy, the changes in NK cell counts were compared with decreases in rat AIA disease activity per unit exposure. How NK cell counts and disease activity respond to changes in AUC exposure are shown in FIG. 7.

On the basis of these rat models, Compound 1 is predicted to decrease NK cell counts by a maximum of approximately 40% over the intended clinical exposure range.

These results demonstrate a quantitative relationship between an endpoint that is directly Jak kinase-dependent, and an endpoint that is mechanistically more distal. The advantage of the latter is that it is agonist-independent and logistically more convenient. Describing this relationship is important because it decreases the risk that the distal endpoint (peripheral NK cell counts) is unrelated to the Jak mechanism of action. Peripheral NK cell count is a mechanistically relevant pharmacodynamic biomarker, because it can be measured in an accessible compartment (peripheral blood) and at the same time as other clinical sample collections, such as those for drug level measurements.

### Example 5

### Pharmacokinetics and Product Metabolism

### Compound 1 Pharmacokinetic Parameters After a Single Dose

The Compound 1 PK profile in monkey and dog was characterized by moderate clearance values (range=0.66 L/hr·kg [dog]−1.3 L/hr·kg [monkey]), with higher clearance in the rat (CLp=2.0-3.8 L/hr·kg; Table 3). Volumes of distribution were high in all species (Vss=1.6-2.7 L/kg). The Compound 1 plasma elimination half-life after IV dosing was shortest in rat (t½=1 hr) and monkey (t½=1.2 hr), with slightly longer values in dog (t½=3.1 hr); plasma elimination half-lives after oral dosing tended to be slightly longer than those observed after intravenous dosing, with values typically in the 3- to 5-hour range.

Compound 1 was slowly absorbed from solution formulations, with peak concentrations noted 1 to 6 hours after dosing. Bioavailability in rats was moderate (30.5%), with higher values noted in both monkey (59.3%) and dog (76.8%). In dogs dosed with a solution formulation, Cmax values were approximately 3-fold lower in fed animals, with AUC values approximately 40% lower when compared with values obtained from the same formulation administered to fasted animals. Solutions of the tartrate salt provided Cmax and AUC values comparable to those obtained from solution formulations of the free base following oral dosing in dog (0.5-2 mg base/kg doses). In the rat, exposures from suspensions of the tartrate salt were also comparable to those obtained with the free base.

### Compound 1 Pharmacokinetic Parameters After Repeated Dose

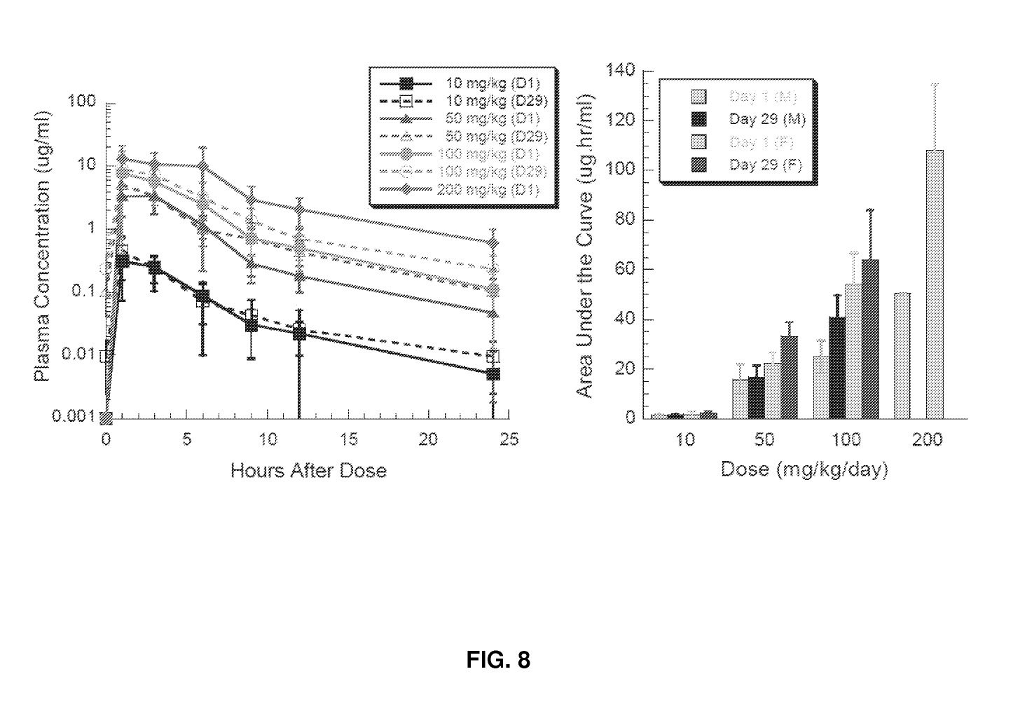

Compound 1 plasma concentrations in female rats tended to be higher than those obtained from male rats receiving 10, 50, 100, or 200 mg/kg/day oral doses for 29 consecutive days; plasma concentrations were not obtained from animals in the 200 mg/kg/day treatment group on Day 29. All dose groups were characterized by a high degree of animal to animal variability. Compound 1 plasma concentrations on Day 29 were comparable to or higher than those measured on the first day of dosing (Day 1). At steady state, the Cmax and AUC values in the 50 and 100 mg/kg/day treatment groups were roughly proportional to the dose, with values in the 10 mg/kg/day treatment group lower than should have been predicted from the other 2 treatment groups (Table 4, FIG. 8).

Compound 1 peak plasma concentrations in male/female rats averaged 0.33/0.65, 2.32/8.85, 5.72/14.9 μg/mL following 29 days of 10, 50, 100 mg/kg/day doses, respectively; AUC values in the same treatment groups averaged 1.49/2.28, 16.8/33.2, and 40.8/63.8 μg·hr/mL.

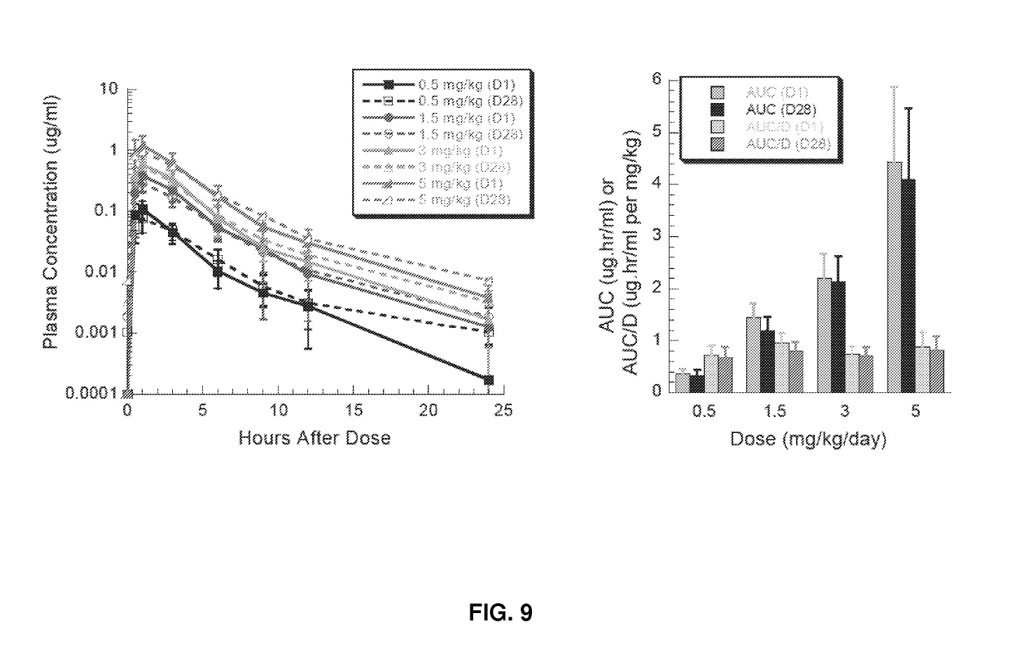

There were no consistent sex differences in the Compound 1 plasma concentrations following 0.5, 1.5, 3, or 5 mg/kg/day oral doses in dogs, with concentrations on Day 28 comparable to those measured after the first dose (Day 1; Table 5, FIG. 9). AUC values were roughly proportional to all doses in this study. Compound 1 peak plasma concentrations on Day 28 averaged 0.076, 0.297, 0.547, and 1.02 μg/mL following 0.5, 1.5, 3, and 5 mg/kg doses, respectively; AUC values averaged 0.339, 1.20, 2.14, and 4.10 μg·hr/mL in the same treatment groups.

### Absorption

MDCK-MDR1 (Transporters)

The permeability of Compound 1 was evaluated using MDCK-MDR1 model at a single concentration with a pan-transporter inhibitor, cyclosporine A. The permeability of Compound 1 was 11.5×10⁻⁶ cm/s, representative of moderately high permeability and the expectation that it should be well absorbed in human. Efflux of Compound 1 by P-gp (MDR1) or BCRP was assessed in a bidirectional MDCKII-MDR1 and -BCRP assay. A net efflux ratio of 2.0 and 3.1 (from 2 independent experiments) in the bidirectional MDCKII-MDR1 assay indicates that Compound 1 is a P-gp substrate. A net efflux ratio of 2.7 and 3.4 (from 2 independent experiments) in the bidirectional MDCKII-BCRP assay indicates that Compound 1 is a BCRP substrate.

Compound 1 was a poor inhibitor of both P-gp and BCRP, with an IC₅₀ of 348 and 126 μM, respectively.

In vivo Absorption

In bile duct-cannulated Sprague-Dawley male rats given orally a single dose of [¹⁴C]Compound 1 (3 mg/kg), at least 63.7% of the dose was absorbed as indicated by the sum of radioactivity recovered in bile and urine, consistent with the high permeability and bioavailability noted above.

### Distribution

Plasma Protein Binding, Binding Site, Red Blood Cell Distribution

In vitro data showed that Compound 1 has moderate to low plasma protein binding across species: 64% (rat), 39% (monkey), 18% (dog), and 47% (human). The mean blood to plasma concentration ratios ranged from 1.27 to 1.48 across species, suggesting Compound 1 was distributed slightly higher in blood than in plasma in rat, dog, monkey, and human.

### Tissue Distribution Studies

Following a 3 mg/kg intravenous dose of [¹⁴C]Compound 1 to male Sprague-Dawley rats, the dosed radioactivity was well distributed into liver and kidneys and skin (tissue-to-plasma ratio [T/P]≧1 at 1 hour after dosing), but distributed poorly into lung, muscle, testes, lymph nodes, heart, and fat (T/P<1). In all selected tissues, the maximum radioactivity concentrations were observed at 1 hour after dosing, and then the radioactivity declined over time. There was no tissue-specific retention of radioactivity.

### Metabolism

The metabolic pathway of Compound 1 is depicted in the scheme below.

TABLE 3
Compound 1 Pharmacokinetics Following a Single Dose in Rat,
Monkey, or Dog
Intravenous Dose

| | | t_{1/2} | V_{c} | Vₛₛ | V_{β} | AUC_{0-∞} | CLₚ | |
|---|---|---|---|---|---|---|---|---|
| Species | Dose^{a} | (hr) | (L/kg) | (L/kg) | (L/kg) | (μg · hr/mL) | (L/hr · kg) | n |
| Rat | 0.3 | 1.0 | 1.8 | 2.7 | 5.7 | 0.08 (0.016) | 3.8 (0.7) | 2 |
| | 3 | 3.0 | 0.46 | 2.2 | 9.4 | 1.55 (0.23) | 2.0 (0.3) | 3 |
| Monkey | 0.1 | 1.1 | 1.2 | 1.9 | 2.2 | 0.072 (0.010) | 1.4 (0.20) | 3 |
| | 1.0 | 1.3 | 1.0 | 1.6 | 2.2 | 0.947 (0.296) | 1.2 (0.44) | 6 |
| Dog | 1 | 3.1 | 1.9 | 2.6 | 2.9 | 1.61 (0.55) | 0.66 (0.22) | 2 |

Oral Dose

| | | t_{1/2} | Cₘₐₓ | Tₘₐₓ | AUC_{0-∞} | F | |
|---|---|---|---|---|---|---|---|
| Species | Dose^{a} | (hr) | (μg/mL) | (hr) | (μg · hr/mL) | (%) | n |
| Rat | 3 | 5.2 | 0.095 (0.022) | 2.2 (1.4) | 0.472 (0.184) | 30.5 (11.8) | 3 |
| Monkey | 1 | 3.3 | 0.134 (0.055) | 1.9 (0.9) | 0.562 (0.171) | 59.3 (18.1) | 6 |
| | 10 | 5.9 | 0.960 (0.566) | 4.3 (1.5) | 4.95 (1.17) | 52.3 (12.4) | 3 |
| Dog | 1 | 2.8 | 0.349 (0.053) | 1.0 (0.0) | 1.24 (0.35) | 76.8 (21.9) | 3 |
| | 5 (f) | 3.7 | 1.53 (0.16) | 1.5 (0.0) | 5.31 (0.97) | 65.8 (12.1) | 3 |
| | 5 (fed) | 5.0 | 0.504 (0.152) | 2.8 (1.3) | 3.23 (0.58) | 40.0 (7.1) | 3 |

Data provided as mean (standard deviation).
^{a}Dose in mg/kg; f = fasted conditions; fed = food provided 30 minutes prior to dosing.

TABLE 4
Compound 1 Pharmacokinetics Following Multiple Oral Dosing in
Sprague-Dawley Rat

| Gp | Dose | Day | M/F | Cₘₐₓ | Cₘₐₓ/D | Tₘₐₓ | AUC | AUC/D |
|---|---|---|---|---|---|---|---|---|
| 2 | 10 | 1 | M | 0.224 (0.058) | 0.022 | 2.2 (1.1) | 1.39 (0.44) | 0.139 |
| | | | F | 0.468 (0.290) | 0.047 | 1.8 (1.1) | 1.87 (0.87) | 0.187 |
| | | 29 | M | 0.329 (0.172) | 0.033 | 1.8 (1.1) | 1.49 (0.43) | 0.149 |
| | | | F | 0.652 (0.269) | 0.065 | 1.0 (0.0) | 2.28 (0.52) | 0.228 |
| 3 | 50 | 1 | M | 3.03 (1.63) | 0.061 | 2.6 (0.9) | 15.9 (6.08) | 0.319 |
| | | | F | 5.16 (1.73) | 0.103 | 2.2 (1.1) | 22.4 (4.13) | 0.449 |
| | | 29 | M | 2.32 (0.70) | 0.046 | 2.6 (0.9) | 16.8 (4.33) | 0.335 |
| | | | F | 8.85 (2.79) | 0.177 | 1.4 (0.9) | 33.2 (5.69) | 0.663 |
| 4 | 100 | 1 | M | 3.91 (1.03) | 0.039 | 3.2 (1.8) | 24.9 (6.66) | 0.249 |
| | | | F | 13.5 (6.29) | 0.135 | 1.4 (0.9) | 54.1 (12.7) | 0.541 |
| | | 29 | M | 5.72 (2.25) | 0.057 | 3.2 (1.8) | 40.8 (8.81) | 0.408 |
| | | | F | 14.9 (8.34) | 0.149 | 2.6 (3.6) | 63.8 (20.4) | 0.638 |
| 2 | 200 | 1 | M | 7.25 (1.61) | 0.036 | 1.7 (1.2) | 50.4 (0.12) | 0.252 |
| | | | F | 18.2 (8.81) | 0.091 | 1.0 (0.0) | 108 (26.5) | 0.539 |
| | | 29 | M | -,- | -,--- | -,- | ---,- | -,---- |
| | | | F | -,- | -,--- | -,- | ---,- | -,---- |

Dose (Compound 1 in mg base/kg/day);
Cₘₐₓ (μg/mL);
Cₘₐₓ/D (μg/mL per mg/kg/day);
Tₘₐₓ (hr);
AUC (μg · hr/mL);
AUC/D (μg · hr/mL per mg/kg/day);
Mean (SD) Data provided from Study TA11-224.²

TABLE 5
Compound 1 Pharmacokinetics Following Multiple Oral Dosing in Dog

| Gp | Dose | Day | M/F | Cₘₐₓ | Cₘₐₓ/D | Tₘₐₓ | AUC | AUC/D |
|---|---|---|---|---|---|---|---|---|
| 2 | 0.5 | 1 | M/F | 0.111 (0.040) | 0.222 | 1.2 (0.8) | 0.359 (0.097) | 0.719 |
| | | 28 | M/F | 0.076 (0.031) | 0.153 | 1.5 (0.9) | 0.339 (0.104) | 0.678 |
| 3 | 1.5 | 1 | M/F | 0.404 (0.156) | 0.270 | 2.0 (2.3) | 1.44 (0.274) | 0.962 |
| | | 28 | M/F | 0.297 (0.085) | 0.198 | 1.1 (0.6) | 1.20 (0.260) | 0.803 |
| 4 | 3 | 1 | M/F | 0.617 (0.191) | 0.206 | 1.3 (0.7) | 2.20 (0.474) | 0.732 |
| | | 28 | M/F | 0.547 (0.091) | 0.182 | 0.9 (0.2) | 2.14 (0.483) | 0.715 |
| 2 | 5 | 1 | M/F | 1.24 (0.505) | 0.247 | 1.4 (1.5) | 4.43 (1.45) | 0.886 |
| | | 298 | M/F | 1.02 (0.590) | 0.204 | 1.0 (0.7) | 4.10 (1.36) | 0.821 |

Dose (Compound 1 dose in mg base/kg);
Cₘₐₓ (μg/mL);
Cₘₐₓ/D (μg/mL per mg/kg/day);
Tₘₐₓ (hr);
AUC (μg · hr/mL);
AUC/D (μg · hr/mL per mg/kg/day)
Data provided as Mean (SD) from TB11-225.³

In vitro Metabolism

The metabolic stability of Compound 1 was evaluated in liver microsomes (0.5 μM) and hepatocytes (1 μM) across species at a single concentration. The intrinsic clearance in liver microsomes (scaled to liver weight/body) of Compound 1 was 5.08 L/hr/kg in rat, <1.6 L/hr/kg in monkey and <2.0 L/hr/kg in dog. The intrinsic clearance of Compound 1 in hepatocytes was 4.07 L/hr/kg in rat, 0.413 L/hr/kg in monkey and 0.415 L/hr/kg in dog.

In human liver microsomes and hepatocyte incubations, Compound 1 showed low intrinsic clearance (1.87 and 0.366 L/h/kg, respectively). The potential for [¹⁴C]Compound 1 to be metabolized by CYP enzymes and flavin monooxygenases was evaluated using recombinant systems. The enzymes identified to potentially contribute to metabolism of [¹⁴C]Compound 1 on the basis of percentage of parent remaining were CYPs 3A4 (36.0%), 2D6 (73.4%), and 3A5 (80.6%). The percentage of parent remaining for all other CYPs was greater than 98.0%.

The in vitro metabolism of [¹⁴C]Compound 1 was investigated using liver cytosol and hepatocytes from rat, dog, monkey, and human, as well as recombinant human cytochrome P450 enzymes (CYP 3A4, 3A5, and 2D6). In vitro biotransformation of Compound 1 primarily occurred at imidazolpyrrolopyrazinyl ethyl pyrrolidine moiety. No metabolism of [¹⁴C]Compound 1 was found in liver cytosol incubations. In hepatocytes, turnover of [¹⁴C]Compound 1 was very low (<10%). CYP3A4 primarily metabolized Compound 1 in vitro to form the major metabolite M11 via oxidation at the pyrrolo pyrazine moiety, followed by ring opening. A minor contribution of CYP2C9 and CYP2D6 was also observed.

### Elimination

Mean total recovery of dosed radioactivity was 96.0% after intravenous administration or 96.8% after oral administration. Drug-related radioactivity was primarily excreted into bile (49.7% of intravenous dose and 52.6% of oral dose), followed by urine (23.7% of intravenous dose and 11.1% of oral dose). In total, after intravenous administration, 25% and 19% of parent drug was excreted unchanged in bile and urine, respectively. After oral administration, 19% and 9% of parent drug was excreted unchanged in the bile and urine, respectively.

### Pharmacokinetic-Drug Interactions

Inhibition

The potential for Compound 1 to inhibit CYP1A2, 2B6, 2C8, 2C9, 2C19, 2D6, or 3A4 (midazolam/testosterone) was evaluated in human liver microsomes using probe substrates. Compound 1 did not inhibit any of the tested isoforms, 1A2, 2B6, 2C8, 2C9, 2C19, 2D6, or 3A4 (midazolam/testosterone), at a concentration up to 30 μM. No obvious time-dependent inhibition of CYP1A2, 2C8, 2C9, 2C19, 2D6, 2B6, and 3A4 was observed in human liver microsomes.

Induction

Compound 1 was evaluated as a potential inducer of CYP1A2, 2B6, and 3A4 at mRNA levels in plated cryopreserved human hepatocytes at concentrations 1, 3, and 10 μM. Compound 1 did not increase mRNA expression of CYP1A2 and 3A4. Compound 1 did not increase mRNA expression of CYP2B6 at 1 and 3 μM, and slightly increased CYP2B6 mRNA expression at 10 μM to 25% of the response of phenobarbital.

Transporters

Compound 1 is an OATP1B1, OAT3, MATE1 and MATEK inhibitor with IC₅₀ values of 48, 43, 11, and >15 μM, respectively. No significant inhibition of OATP1B3, OCT1, OCT2, or OAT1 uptake was observed at up to 30 μM Compound 1.

Drug-Drug Interactions Simulations

Preliminary predictions using physiologically-based PK modeling, suggest that Compound 1, as a CYP3A4/5 substrate, is likely to have a moderate interaction with potent CYP3A inhibitors or inducers such as ketoconazole and rifampicin, respectively.

### Example 6

### Toxicology

The toxicologic profile of Compound 1 was evaluated in nonclinical toxicity studies that included a 4-week, non-GLP oral toxicity study in dogs at a single dose level of 5 mg/kg/day as well as pivotal, GLP-compliant oral toxicity studies in both rats and dogs. The GLP-compliant studies each included 4 weeks of oral dosing followed by a 4-week recovery period in both rats and dogs followed by GLP-compliant chronic toxicity studies in rats (6 months of oral dosing) and dogs (9 months of oral dosing). Compound 1 has also been evaluated in a standard battery of in vitro genotoxicity studies and an in vivo rat bone marrow micronucleus test. Definitive GLP embryofetal development studies with Compound 1 in rats and rabbits have also been completed.

Beagle dogs were used as the non-rodent toxicology species for Compound 1 because of good oral bioavailability. Rats were selected as the rodent species on the basis of PK and metabolism. In all in vivo nonclinical studies to date, formulated Compound 1 (as free base for the in vivo rat bone marrow micronucleus test and the 4-week GLP studies in rats and dogs and as Compound 1 tartrate in the 6-month rat and 9-month dog GLP studies and the definitive embryofetal development studies) was dosed orally once daily, with doses expressed as milligrams of free base per kilogram of body weight per day (mg/kg/day).

In the pivotal 4-week rat study with Compound 1, dose-limiting effects included mortality at 100 and 200 mg/kg/day. Only mild decreases in red cell mass (−13%, −12%, and −14% compared to control means for RBC counts, hemoglobin concentration, and hematocrit, respectively) were present in rats following 4 weeks of administration of 100 mg/kg/day Compound 1, and these decreases were not present following the 4-week recovery period. Non-adverse, dose-dependent decreases in circulating lymphocytes (to −71% compared to controls) were present in rats at 10, 50, and 100 mg/kg/day and demonstrated partial recovery at the end of the 4-week recovery period. The NOAEL in the rat was 50 mg/kg/day Compound 1, which resulted in a Cₘₐₓ of 5.59 μg/mL and AUC₀₋₂₄ of 25.0 μg·hr/mL (values combined for males and females).

In the subsequent 6-month chronic rat study with Compound 1, the NOAEL was determined to be 20 mg/kg/day in males and females, resulting in Cₘₐₓ values of 1.11 and 2.24 μg/mL, respectively, and AUC₀₋₂₄ values of 3.83 and 6.84 μg·hr/mL, respectively.

Adverse findings consisted of minimal to moderate tubular degeneration/regeneration in the kidneys of rats only at 50 mg/kg/day. Non-adverse findings were present in the lymphoid tissues (spleen, thymus, and lymph nodes) at 5, 20, and/or 50 mg/kg/day. In addition, mild to moderate decreases in total WBCs, due primarily to decreases in lymphocytes, were observed in both sexes at all doses of Compound 1. Mild decreases in red cell mass and reticulocytes were observed in both sexes, mainly at 50 mg/kg/day. These mild decreases in red cell mass were considered non-adverse based on magnitude.

In both the non-GLP 4-week dog study and the GLP 4-week dog study, no in-life findings related to administration of Compound 1 were observed. Adverse effects in dogs following 4 weeks of oral dosing at 3 and 5 mg/kg/day in the pivotal, GLP-compliant study included decreases in red cell mass (RBC counts, hemoglobin concentration, and hematocrit) and microscopic findings in popliteal lymph nodes (mixed cell infiltrates with extension into pericapsular tissues). Decreases in red cell mass correlated with decreases in reticulocytes and showed increases in severity with longer duration of dosing, consistent with suppression of erythropoiesis. Decreases in circulating reticulocytes were reversible after the first 2 weeks of the 4-week recovery period, while individual red cell parameters (RBC counts, hemoglobin concentration, hematocrit) returned to near-baseline values by the end of the recovery period in the pivotal 4-week toxicity study. Non-adverse decreases in red cell mass were present in males administered 1.5 mg/kg/day (to −18% compared with baseline values in 2 of 12 animals), and minimal mixed cell infiltrates in popliteal lymph nodes (considered non-adverse) were present in 1 animal at 1.5 mg/kg/day and 1 animal at 0.5 mg/kg/day. Following the 4-week recovery period, mixed cell infiltrates in popliteal lymph nodes persisted in recovery animals administered 5 mg/kg/day but were not present in animals administered 1.5 mg/kg/day, consistent with reversibility only at 1.5 mg/kg/day. Mild non-adverse decreases (from −20% to −37% compared with baseline values) in circulating lymphocytes were observed in animals administered 5 mg/kg/day Compound 1, with evidence of reversibility after the first 2 weeks of the 4-week recovery period.

Therefore, 1.5 mg/kg/day Compound 1 free base was established as the NOAEL in the dog and resulted in a Cₘₐₓ of 0.297 μg/mL and an AUC₀₋₂₄ of 1.2 μg·hr/mL (values combined for males and females).

In the subsequent 9-month chronic toxicity study in dogs, there were no adverse findings at daily dosages up to 1.5 mg/kg/day. The only direct effect of Compound 1 administration was a minimal to mild, non-adverse decrease in red cell mass at 0.5 and 1.5 mg/kg/day from Dosing Day 28 to 182. At the end of dosing (Day 265), decreases in red cell mass were either similar to or less than decreases reported on Day 91. Based on the magnitude of change and/or improvement with continued dosing, these decreases in red cell mass were not considered adverse. In contrast to the 1 month study, microscopic findings in popliteal lymph nodes (mixed cell infiltrates with extension into pericapsular tissues) were not detected above the level detected in dogs treated with vehicle alone. Other secondary effects related to the possible immunomodulation due to administration of Compound 1 were considered non-adverse.

Based on these findings, the NOAEL was 1.5 mg/kg/day, which resulted in an average (male and female) Day 272 Cₘₐₓ of 212 ng/mL and AUC of 888 ng·hr/mL.

TABLE 6
List of Toxicology Studies Conducted with Compound 1

| | | | | | |
|---|---|---|---|---|---|
| | | | | Dosage | NOAEL |
| Study | Species | Route | Duration | (mg/kg/day) | (mg/kg/day) |

Repeated Dose

| | | | | | |
|---|---|---|---|---|---|
| Screening toxicity study | Dog | Oral (liquid in capsule) | 4 weeks | 5 | None |
| Pivotal toxicity study (GLP) | Rat | Oral (gavage) | 4 weeks | 0, 10, 50, 100, 200 | 50 |
| Pivotal toxicity study (GLP) | Dog | Oral (liquid in capsule) | 4 weeks | 0, 0.5, 1.5, 3, 5 | 1.5 |
| Pivotal toxicity study (GLP) | Rat | Oral (gavage) | 26 weeks | 0, 5, 20, 50 | 20 |
| Pivotal toxicity study (GLP) | Dog | Oral (liquid in capsule) | 39 weeks | 0, 0.1, 0.5, 1.5 | 1.5 |

Embryofetal Development

| | | | | | |
|---|---|---|---|---|---|
| Dose range-finding study | Rat | Oral | GD 6-17 | 0, 50, 100, 150 | —^{a} |
| Definitive EFD toxicity study | Rat | Oral | GD 6-17 | 0, 5, 20, 50 | NOEL < 5 |
| Dose tolerability study | Rabbit | Oral | GD 7-19 | 0, 50, 100, 150, 200 | —^{a} |
| Dose range-finding study | Rabbit | Oral | GD 7-19 | 0, 5, 20, 50 | —^{a} |
| Definitive EFD toxicity study | Rabbit | Oral | GD 7-19 | 0, 2.5, 10, 25 | NOEL = 10 |

EFD = embryofetal development,
GD = gestation day;
GLP = Good Laboratory Practices;
NOEL = no-observed-effect-level
^{a}Not evaluated

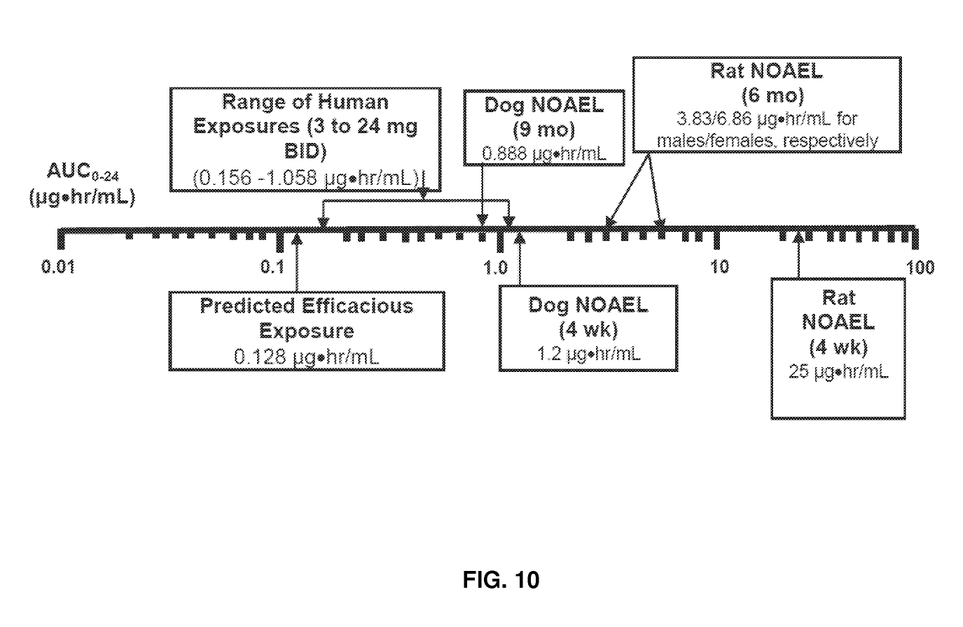

A summary of plasma exposures for predicted efficacious exposure, range of human exposures, and non-clinical NOAELs for dog and rat toxicity studies with Compound 1 is presented in FIG. 10.

A summary of all toxicology studies conducted with Compound 1 is presented in Table 6.

Oral toxicity of Compound 1 was evaluated in non-clinical toxicity studies that included a non-GLP, 4-week oral toxicity study in dogs at a single dose level of 5 mg/kg/day and pivotal, GLP-compliant oral toxicity studies in both rats and dogs. The GLP-compliant studies included 4 weeks of oral dosing followed by a 4-week recovery period in both rats and dogs followed by GLP-compliant chronic toxicity studies in rats (6 months of oral dosing) and dogs (9 months of oral dosing).

TABLE 7
Summary of Repeated-Dose Studies in Rats and Dogs

| | | | Dosage | Number | | |
|---|---|---|---|---|---|---|
| Species | Duration | GLP | mg/kg/day | Sex/Group | NOAEL | Adverse Findings |
| Dog | 4 weeks | No | 5 | 2 | None | 5: Popliteal lymph |
| | | | | | | node - mixed cell infiltrates |
| Rat | 4 weeks | Yes | 0, 10, 50, 100, 200 | 10 (5)^{a} | 50 | ≧100: Mortality |
| | | | | | | 100: Kidneys-cortical tubular |
| | | | | | | degeneration/regeneration, |
| | | | | | | Liver-necrosis (early death |
| | | | | | | animals only) |
| Dog | 4 weeks | Yes | 0, 0.5, 1.5, 3, 5 | 4 (2)^{a} | 1.5 | ≧3: Popliteal lymph |
| | | | | | | nodes - mixed cell infiltrates |
| | | | | | | with extension into pericapsular |
| | | | | | | tissues, decreases in red cell |
| | | | | | | mass (RBC counts, hemoglobin |
| | | | | | | concentration, hematocrit) |
| Rat | 26 weeks | Yes | 0, 5, 20, 50 | 20 | 20 | 50: Kidneys-tubular |
| | | | | | | degeneration/regeneration |
| Dog | 39 weeks | Yes | 0, 0.1, 0.5, 1.5 | 4 | 1.5 | None |

RBC = red blood cell
^{a}Number in parentheses represents the number of designated recovery animals.

Rodent Studies

The 4-week rat study was conducted at dose levels of 0, 10, 50, 100, and 200 mg/kg/day by daily oral administration (gavage). Designated recovery animals in the groups administered 0, 50, and 100 mg/kg/day Compound 1 were maintained for an additional 4 weeks without dosing. At 200 mg/kg/day Compound 1, mortality of several animals on Dosing Day 1 led to termination of the entire dose group on Dosing Day 2 and Dosing Day 3 without histopathology evaluation. The cause of death was not determined for animals that died. At 100 mg/kg/day, 5 males were found dead or were euthanized because of clinical condition on Dosing Days 1, 3, 4, 18, and 26. Adverse histopathology findings in animals administered 100 mg/kg/day included minimal to marked degeneration/regeneration of the renal cortical tubular epithelium. Correlating findings in clinical chemistry parameters consisted of mild increases in mean phosphorus in males and females at 100 mg/kg/day (up to +10% compared with the control mean value) at the end of the dosing period. The histopathology finding in the kidney and the increases in serum phosphorus were not present in animals that survived to the end of the 4-week recovery period, consistent with reversibility. Moderate to marked multifocal, midzonal, or diffuse liver necrosis was also present at 100 mg/kg/day, but only in early death animals (3 of 5) in this dose group. Adverse pathology findings were not present in animals administered 10 or 50 mg/kg/day.

Therefore, the NOAEL in the rat was 50 mg/kg/day Compound 1, which resulted in a Cₘₐₓ of 5.59 μg/mL and AUC₀₋₂₄ of 25.0 μg·hr/mL (values combined for males and females).

Non-adverse findings in the 4-week rat study included mild dose-dependent decreases in circulating lymphocytes (to −71% compared with controls) at 10, 50, and 100 mg/kg/day Compound 1 that demonstrated partial recovery by the end of the 4-week recovery period.

Mild decreases (−13%, −12%, and −14% compared to control means for RBC counts, hemoglobin concentration, and hematocrit, respectively) in red cell mass (considered not adverse) were present only in females administered 100 mg/kg/day Compound 1, and these decreases demonstrated recovery following the 4-week recovery period. Non-adverse histopathology findings included decreased numbers of lymphocytes in thymus in rats at 50 and 100 mg/kg/day, decreased numbers of lymphocytes in the marginal zones in the spleen in males at 100 mg/kg/day, and minimal to mild hypocellularity of bone marrow in rats at 100 mg/kg/day. These findings for thymus, spleen, and bone marrow were not observed in animals that survived to the end of the 4-week recovery period. The findings in lymphoid tissues were consistent with expected pharmacologic effects of Compound 1.

The 6-month chronic rat study was conducted at dose levels of 0, 5, 20, and 50 mg/kg/day by daily oral administration (gavage). A non-adverse, dose-related decrease (relative to control) in body weight gain observed in males at 20 and 50 mg/kg/day (10% and 15%, respectively, at the end of study) corresponded to decreased food consumption first noted during the third week of dosing and that continued throughout the dosing period.

Adverse findings attributed to Compound 1 administration for 6 months in rats consisted of minimal to moderate tubular degeneration/regeneration in the kidneys at 50 mg/kg/day. Non-adverse findings were present in the lymphoid tissues (spleen, thymus, and lymph nodes), stomach, and/or tongue at 5, 20, and/or 50 mg/kg/day. Findings in lymphoid tissues consisted of varying degrees of lymphoid depletion, consistent with the expected pharmacologic activity of Compound 1. In the stomach of males and females at 50 mg/kg/day, findings consisted of minimal to mild erosion and ulceration of the mucosa, primarily at the limiting ridge of the non-glandular stomach along with subacute/chronic inflammation, edema, and/or epithelial hyperplasia. One male at 20 mg/kg/day had minimal focal ulceration of the limiting ridge. Due to the minimal to mild magnitude and absence of an anatomic correlate in humans (humans do not have a non-glandular stomach), these findings were considered related to Compound 1 administration but not adverse. Similarly, minimal to mild mucosal erosion was present in the tongue of males and females at 50 mg/kg/day and was considered non-adverse.

Mild decreases in red cell mass and reticulocytes were observed in both sexes, mainly at 50 mg/kg/day. These mild decreases in red cell mass were considered non-adverse based on magnitude. Mild to moderate decreases in total WBC count, due primarily to decreases in lymphocytes, were observed in both sexes at all doses of Compound 1.

Based on the presence of adverse kidney findings only at 50 mg/kg/day, the NOAEL following 6 months of dosing in rats was determined to be 20 mg/kg/day in males and females, resulting in Cmax values of 1.11 and 2.24 μg/mL, respectively, and AUC0-24 values of 3.83 and 6.84 μg·hr/mL, respectively.

Non-Rodent Studies

In a non-GLP, 4-week oral toxicity study in beagle dogs at a single dose level of 5 mg/kg/day, no in-life findings related to administration of Compound 1 were observed.

Only 1 of 4 dogs had mild decreases in red cell mass (RBC counts, hemoglobin concentration, hematocrit), but these decreases were considered not adverse because of their mild severity.

The GLP-compliant 4-week dog study was conducted in beagle dogs at dosages of 0, 0.5, 1.5, 3, and 5 mg/kg/day by daily oral capsule administration. In this study, groups of an additional 2 animals per sex in the groups administered 0, 1.5, and 5 mg/kg/day Compound 1 were maintained for an additional 4-week recovery period.

No Compound 1-related clinical observations or changes in food consumption or body weights were present during the dosing or recovery period. At 3 and 5 mg/kg/day, mild to moderate decreases in red cell mass (RBC counts, hemoglobin concentration, and hematocrit) were considered adverse because of the magnitudes of the decreases (up to −23% and −32% at 3 and 5 mg/kg/day, respectively). Decreases in red cell mass correlated with decreases in reticulocytes and showed increases in severity with longer duration of dosing, consistent with suppression of erythropoiesis. Decreases in circulating reticulocytes were reversible after the first 2 weeks of the 4-week recovery period, while individual red cell parameters (RBC counts, hemoglobin concentration, hematocrit) returned to near baseline values by the end of the recovery period in this study. Minimal to mild hypocellularity of the bone marrow was present in males at 3 and 5 mg/kg/day and in females at 5 mg/kg/day and correlated with decreases in circulating reticulocytes. However, this observation was not present at the end of the recovery period, consistent with the reversibility of decreases in red cell mass and circulating reticulocytes.

Decreases in red cell mass were also present in animals at 1.5 mg/kg/day but were considered not adverse because of the low incidence and mild severity of the decreases relative to baseline values (to −18% compared with baseline values in 2 of 12 animals).

All dogs in this study at doses up to 5 mg/kg/day were clinically normal with no Compound 1-related clinical observations or changes in food consumption or body weight and with no evidence of any effect on overall health status.

Affected popliteal lymph nodes at any dose in the study were grossly normal (e.g., not enlarged), and no other lymph nodes examined (mandibular, mesenteric, or tracheobronchial) were affected. There were no apparent changes in hematology parameters that would indicate the presence of a systemic inflammatory response. Furthermore, the incidence and severity of the mixed cell infiltrates in popliteal lymph nodes were clearly dose-related with only 1 of 8 animals at 0.5 mg/kg/day and 1 of 8 animals at 1.5 mg/kg/day having this finding of minimal severity. This finding appeared to be dose-dependent with an increase in incidence and severity that ranged up to moderate with infiltrates extending into the pericapsular tissues at the 3 and 5 mg/kg/day doses. In dogs administered 0.5 or 1.5 mg/kg/day, mixed cell infiltrates were confined to the subcapsular sinus and/or capsule of the popliteal lymph nodes, and there were no structural alterations to normal nodal architecture. Although the mixed cell infiltrates in popliteal lymph nodes persisted in animals administered 5 mg/kg/day after a 4-week recovery period, this finding was not present in animals administered 1.5 mg/kg/day Compound 1. This finding in popliteal lymph nodes would be expected to be reversible at 5 mg/kg/day given a longer recovery period (e.g., 3 months). In summary, the finding of minimal mixed cell infiltrates in the popliteal lymph nodes of dogs administered 0.5 or 1.5 mg/kg/day Compound 1 for 4 weeks, although considered related to administration of Compound 1, is not considered to be an adverse finding.

On the basis of the presence of adverse findings following administration of 3 or 5 mg/kg/day Compound 1 for 4 weeks, 1.5 mg/kg/day was established as the NOAEL in the dog and resulted in a Cₘₐₓ of 0.297 μg/mL and AUC₀₋₂₄ of 1.2 μg·hr/mL (values combined for males and females).

A subsequent GLP-compliant 9-month chronic dog study was conducted in beagle dogs at dosages of 0, 0.1, 0.5, and 1.5 mg/kg/day by daily oral capsule administration. No Compound 1-related changes in food consumption or body weights were observed during the dosing period. All dogs survived to the end of the study although 1 male at 1.5 mg/kg/day had administration of Compound 1 suspended due to clinical findings (increased neutrophil counts, paw swelling, and the presence of demodicosis) that were considered secondary to Compound 1 administration. This animal was maintained on study without dose administration and with necessary veterinary care until the end of the dosing period on Day 275.

In this 9-month dog study, direct Compound 1-related effects were limited to a minimal to mild, generally dose-dependent, decrease in red cell mass (indicated by mean hemoglobin) on Dosing Days 28, 91, and 182 in males (to −18%) and females (to −7%) administered 0.5 or 1.5 mg/kg/day. Near the end of dosing (Dosing Day 265), decreases in individual hemoglobin values were either similar to less than decreases reported on Dosing Day 91 for males and females at 0.5 and 1.5 mg/kg/day. Based on the magnitude of change and/or improvement with continued dosing, the decreases in red cell mass were considered non-adverse.

Minimal to mild decreases in indices of red cell mass (RBC count, hemoglobin and hematocrit) occurred at most time points for 6 of 8 animals at 0.5 mg/kg/day and 7 of 8 animals at 1.5 mg/kg/day, compared to mean individual baseline values. Overall, the decreases were greater in males than in females. For males and females at 1.5 mg/kg/day, the lowest decreases in red cell mass (as indicated by individual hemoglobin values) occurred on Dosing Day 91. For males and females at 0.5 mg/kg/day, decreases in red cell mass generally remained more consistent across time points. At the end of dosing, decreases in individual hemoglobin values were either similar to or less than decreases reported on Dosing Day 91 for males and females at 0.5 and 1.5 mg/kg/day. Based on magnitude and/or improvement with continued dosing, the decreases in red cell mass were not considered adverse at any dose level.

Based on the lack of adverse findings in the 9-month dog study, the NOAEL was determined to be 1.5 mg/kg/day, which resulted in an average (male and female) Day 272 Cₘₐₓ of 212 ng/mL and AUC of 888 ng·hr/mL.

In the definitive GLP rat study, 3 treatment groups of 25 time-mated female CD® (Crl:CD®[SD]) rats/group were administered Compound 1 at respective dose levels of 5, 25, or 75 mg/kg/day. One additional group of 25 time-mated females served as the control and received the vehicle, 0.2% hydroxypropyl methylcellulose (HPMC, high viscosity) in deionized water. The vehicle or Compound 1 was administered to all groups via oral gavage once a day from Gestation Day (GD) 6 to 17.

No Compound 1-related effects were observed in clinical observations, gestation body weights, body weight change, food consumption, or maternal macroscopic examinations.

In the definitive GLP rabbit study, 3 treatment groups of 20 time-mated female New Zealand White Hra:(NZW)SPF rabbits/group were administered Compound 1 at respective dose levels of 2.5, 10, or 25 mg/kg/day. One additional group of 20 time-mated females served as the control and received the vehicle, 0.2% hydroxypropyl methylcellulose (HPMC, high viscosity) in deionized water. The vehicle or Compound 1 was administered to all groups via oral gavage once a day from GD 7 to 19.

At 2.5 and 10 mg/kg/day, there were no Compound 1-related effects observed in clinical observations, gestation body weights or body weight change, food consumption, or macroscopic examinations. Likewise, no effect of treatment at these dose levels was observed on uterine implantation parameters, fetal sex ratios, fetal body weights, or fetal external, visceral, or skeletal examinations.

### Example 7

### Preclinical Reticulocyte Effects of Compound 1 vs. Tofacitinib

To determine the potential effect of Compound 1 administration on reticulocyte development, a side-by-side experiment was carried out to compare Compound 1 with the commercially available FDA approved Rheumatoid Arthritis (RA) drug Tofacitinib.

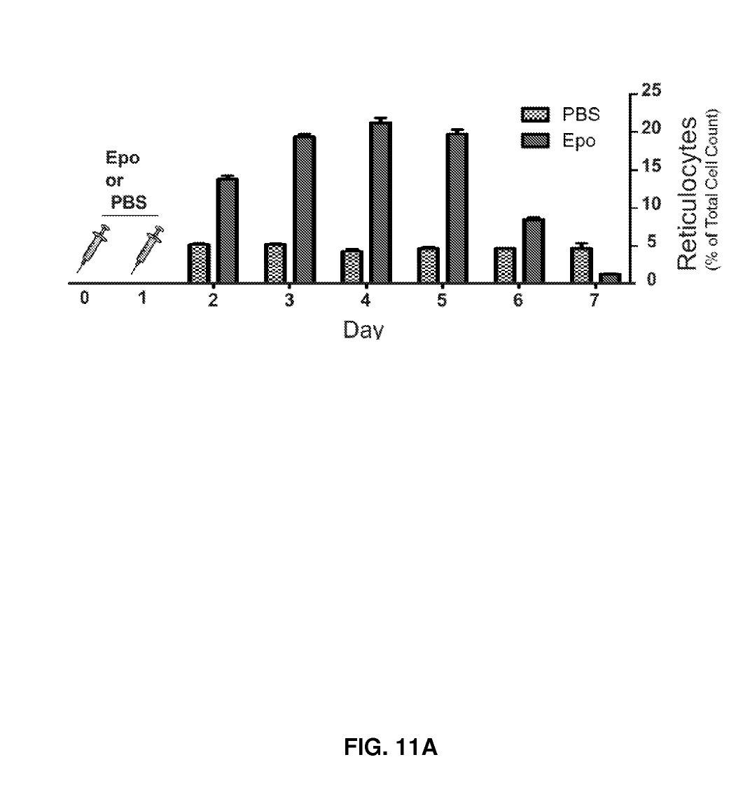

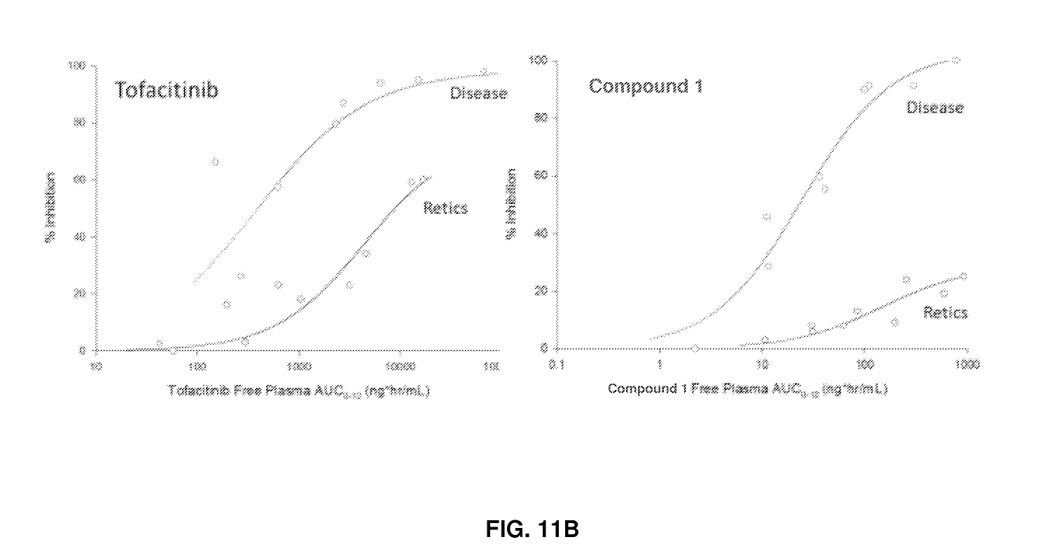

FIG. 11A shows that erythropoietin (EPO) injected i.v. on Day 0 and 1 resulted in modest but precise reticulocytosis in rats, peaking around Days 3-5 post injection, before tailing off on Days 6 and 7 to background level. In contrast, PBS control has negligible (if any) effect. This is consistent with the fact that EPO signals through Jak2 to stimulate reticulocyte development.

Also consistent with the results shown in Table 1, Compound 1 is about 74-fold more selective for Jak1 inhibition over Jak2 inhibition, while Tofacitinib is only about 24-fold more selective. As a result, FIG. 11B shows that the effects of Compound 1 on reticulocyte deployment are less than that of Tofacitinib over the exposure range efficacious in the rat AIA disease model. The slope of curve representing reticulocyte development inhibition in the left panel (for Tofacitinib) is much steeper than the one in the right panel (Compound 1), suggesting that a much higher therapeutic efficacy (or “% inhibition” for disease) can be achieved using Compound 1 without a significant increase of side effect such as reticulocyte development inhibition.

Similarly, FIG. 11C shows that reticulocyte deployment at efficacious exposures is closely related to Jak1/Jak2 selectivity. Here, therapeutic efficacy, as measured by “% inhibition of Paw Swelling on Last Day” in the rat AIA model (previously described), is plotted against extent of undesired side effect as measured by “% inhibition of reticulocytes,” for Tofacitinib, Baricitinib (another Jak inhibitor as RA drug candidate currently undergoing clinical trial), and Compound 1. It is apparent that, as the therapeutic efficacy increases, presumably due to increase of drug concentration, the associated undesired side effect also increases. However, such side effect increase is much more tamed in the case of Compound 1 where the Jak1/Jak2 potency ratio is relatively high at about 74-fold. In contrast, the side effect increases much faster for Tofacitinib, when the Jak1/Jak2 potency ratio is relatively low at about 24-fold. The effect of Baricitinib is between those for Compound 1 and Tofacitinib, which is consistent with its intermediate Jak1/Jak2 potency ratio of about 27-fold. The data shows that the relatively rapid rise in undesirable reticulocyte inhibition has limited the therapeutic efficacy of Tofacitinib to about 60% inhibition of Paw Swelling, while Compound 1 achieved about 80% inhibition at about the same level of reticulocyte inhibition.

### Example 8

### Preclinical NK Cell Effects of Compound 1 vs. Tofacitinib

To determine the potential effect of Compound 1 administration on NK cell counts, a side-by-side experiment was carried out to compare Compound 1 with Tofacitinib.

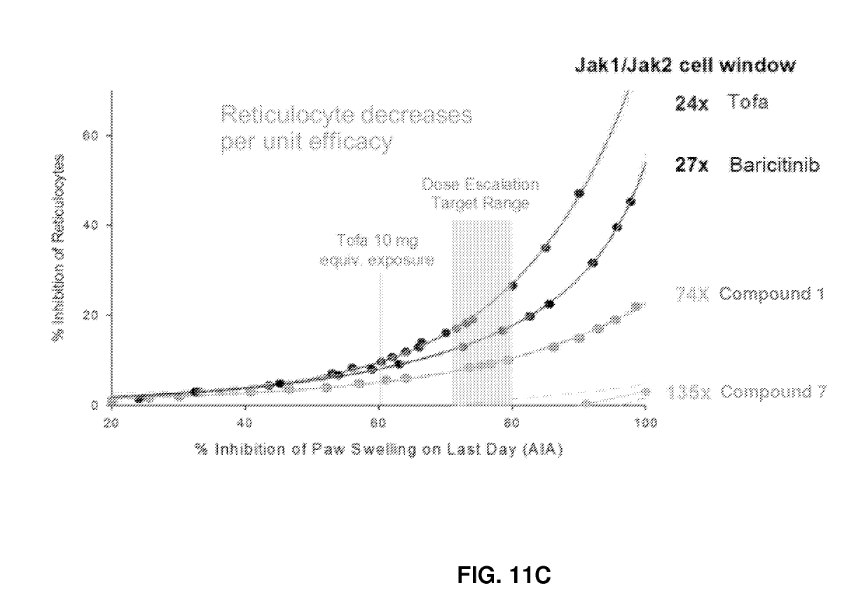

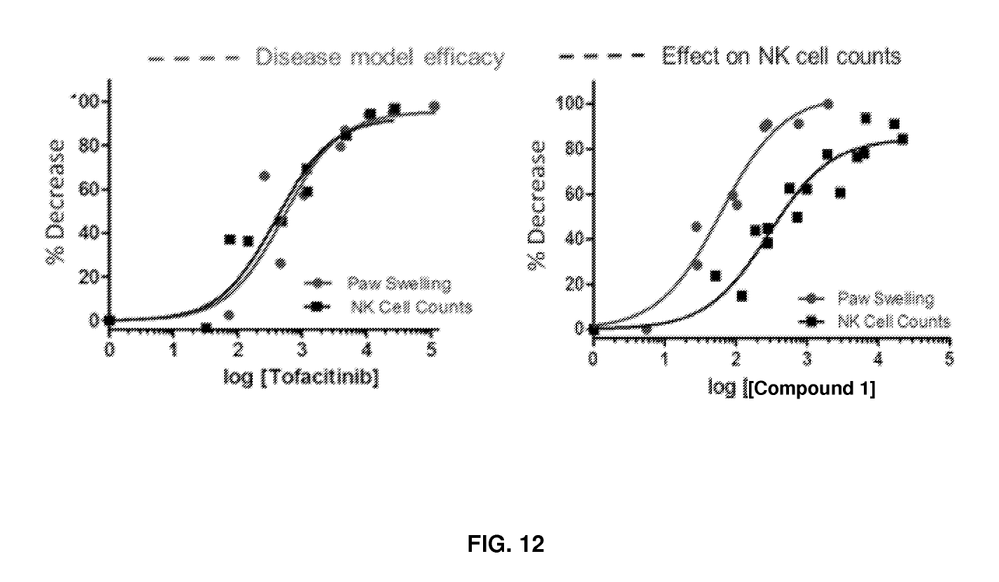

FIG. 12 shows that the effects of Compound 1 on peripheral NK cell counts are less than that of Tofacitinib over the efficacious exposure range. The log concentrations for Tofacitinib and Compound 1 are expressed as AUC exposure (ng·hr/mL).

As the log concentration increases for Tofacitinib, “% Decrease in Paw Swelling” (a measurement for therapeutic efficacy) increases, while at the same time and roughly the same pace, “% Decrease for NK Cell Counts” (a measurement for an undesired side effect) also increases. In contrast, when the log concentration increases for Compound 1, “% Decrease in Paw Swelling” increases significantly before“% Decrease for NK Cell Counts” starts to increases, hence creating a therapeutic window in which therapeutic efficacy is high while side effects (as measured by NK Cell Counts decrease) remain relatively low.

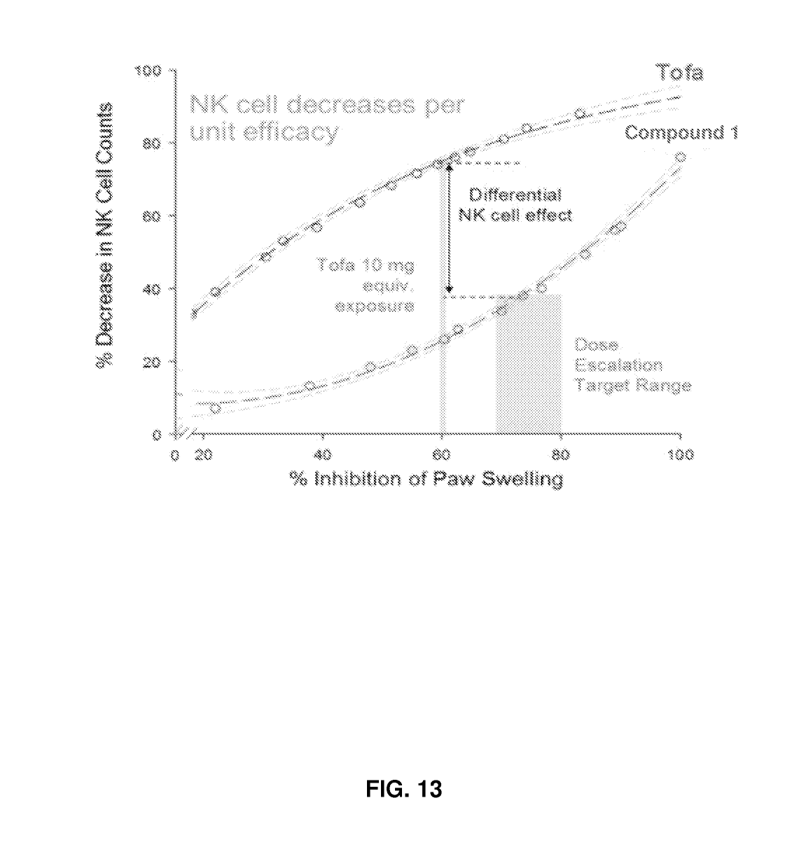

FIG. 13 suggests that high doses of Compound 1 should have relatively low effects on NK cells counts compared to Tofacitinib in view of experimental data from rat. Here, efficacy measure “% inhibition of paw swelling” (from the rat AIA model) is plotted against detrimental side effect “% decrease in NK cell counts,” for both Tofacitinib and Compound 1. It is apparent that NK cell count decreases per unit efficacy increase for both Tofacitinib and Compound 1. However, the rate of increase in Tofacitinib is much more pronounced that that of Compound 1, such that at about 60% efficacy (% inhibition of paw swelling), NK cell count decrease reaches about 80% in Tofacitinib administered at 10 mg dose, while the same only reaches about a relatively harmless 25% in Compound 1. At this level of NK cell count decrease, the Compound 1 dose is lower than the doses currently tested in human subjects.

Since high doses of Compound 1 should have relatively low effects on NK cells counts compared to Tofacitinib, according to the data, it is expected that Compound 1 can be administered at a higher dose level to achieve a higher efficacy level before NK cell counts drop significantly.

Table 8 below shows that Compound 1 spares NK peripheral NK cell counts at efficacious exposures, potentially due to increased potency against IL6 signaling.

TABLE 8
Compound 1 spares peripheral NK cell
counts at efficacious exposures

| | NK | Paw | IL-15 | IL-6 |
|---|---|---|---|---|
| | Counts | Swelling | (pSTAT5, | (pSTAT3, |
| | (ng*hr/mL) | (ng*hr/mL) | CD3⁺, μM) | CD14⁺, μM) |
| Compound 1 | 290 | 62 | 0.016 | 0.017 |
| Tofacitinib | 391 | 514 | 0.022 | 0.114 |
| Fold | 1.3 | 8.2 | 1.4 | 6.7 |

The data in Examples 7 and 8 shows that, despite the subtle (yet robust) potency differences between Compound 1 and tofacitinib in vitro, there were substantial differences in outcomes dependent on specific, relevant cytokine stimuli in vivo. For example, Compound 1 had remarkably lower effects on EPO dependent reticulocyte deployment in healthy rats per unit efficacy than tofacitinib. Similarly, Compound 1 had much lower impact on peripheral NK cell counts than tofacitinib at efficacious exposures. The differential effect on NK cell counts may reflect the relatively low potency of Compound 1 against common gamma chain signaling compared to IL-6 signaling.

According to FDA regulatory documents, tofacitinib dosed in RA patients lead to substantial decreases in peripheral NK cell counts and a concomitant increase in viral infections and malignancies. Moreover, tofacitinib also decreased NK cell counts in transplantation patients and Cynomolgus monkeys (Boric et al., Transplantation 79:791-801, 2005; van Gurp et al., Am. J. Transplant 8:1711-1718, 2008). Because defects in human NK cell function are associated with a characteristic spectrum of infections, including varicella zoster virus, it is expected that sparing effects on NK cells or NK subsets at clinically efficacious exposures would improve the safety and tolerability of Jak inhibitors in RA patients.

### Example 9

### Clinical Ex Vivo NK Cell and Reticulocyte Effects of Compound 1 vs. Tofacitinib

To determine the potential effect of Compound 1 administration on NK cell counts and reticulocyte development in human subjects, a side-by-side experiment was carried out to compare Compound 1 with Tofacitinib.

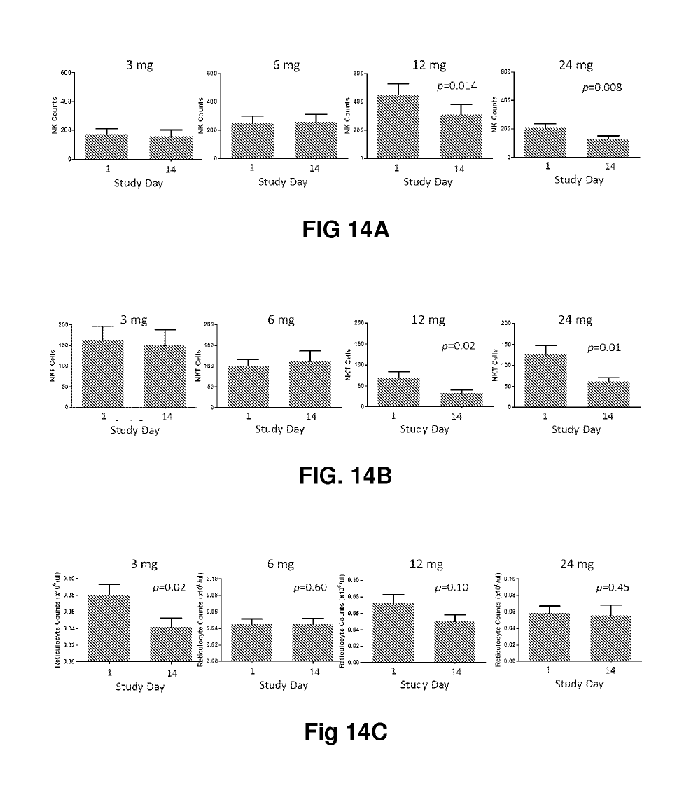

FIGS. 14A and 14B show that Compound 1 dosed bid for 14 days in healthy human subjects reduced peripheral NK or NKT cell counts per μL only at 12 and 24 mg (doses higher than the presumptive therapeutically efficacious dose).

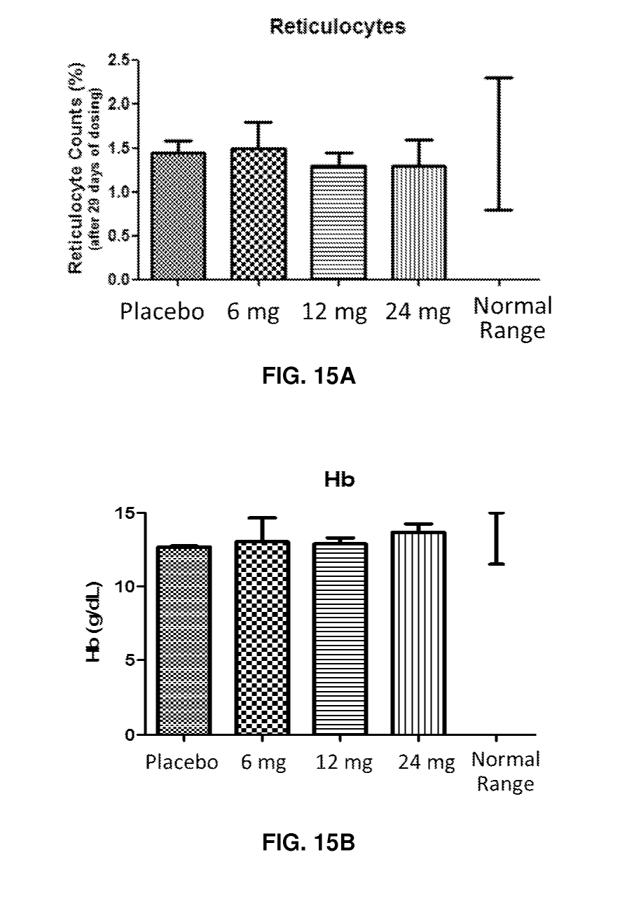

FIG. 14C shows that this same dose range does not dose dependently reduce reticulocyte counts. Consistent with this finding, FIGS. 15A and 15B show that circulating reticulocytes and hemoglobin level were not reduced in RA patients treated twice a day with Compound 1 at 6 mg, 12 mg, and 24 mg for 29 days.

The data suggests that at a dose that is likely efficacious based on Jak1 inhibition, or at a higher dose, Compound 1 does not appreciably reduce reticulocyte count, NK cell count, or NKT cell count, and is thus expected to have negligible undesirable side effect at the efficacious dose, at least with respect to Tofacitinib.

### Example 10

### Effects in Humans

Compound 1 has been studied in 2 Phase 1 studies, first-in human single ascending dose Study M13-401, and then in multiple ascending dose Study M13-845. In a completed single-dose, placebo-controlled, double-blind randomized study designed to study food effect and drug-drug interaction, a total of 54 healthy volunteers have received Compound 1, with 14 healthy volunteers receiving placebo as control. The primary objectives of the study is to assess the safety, tolerability, and PK of single ascending doses of Compound 1, and to evaluate the effects of food and ketoconazole on the safety and PK of Compound 1 in healthy volunteers.

A total of 32 healthy volunteers have also received multiple doses of Compound 1 for 14 days (Study M13-845 Substudy 1).

In addition, 14 patients with RA have been enrolled and completed the double-blind Substudy 2 in Study M13-845. The study is designed as a multiple-dose, randomized, multicenter trial, with the primary objective as assessing the safety, tolerability, and PK of multiple ascending doses of Compound 1 in healthy adult volunteers and to assess the safety, tolerability, and PK of multiple doses of Compound 1 in patients with RA who are on a stable methotrexate regimen.

Details of these studies and results obtained therefrom are provided below.

### Pharmacokinetics

Two Phase 1 studies have been conducted to determine the PK of Compound 1 as single ascending doses in Study M13-401 and as multiple ascending doses in Study M13-845.

Single-Dose Studies

Study M13-401 was a randomized, placebo-controlled, double-blind study in healthy adults that consisted of 2 substudies. In Substudy 1, the PK of Compound 1 was assessed after single escalating oral doses ranging from 1.0 mg to 48.0 mg, and in Substudy 2, the effect of food and ketoconazole on the PK of a single dose of 3.0 mg Compound 1 was assessed.

The dose groups for the Study M13-401 Substudies 1 and 2 are shown in Table 10.

TABLE 10
Dose Groups in Study M13-401

| | | |
|---|---|---|
| | | No. of Subjects |
| Substudy | Regimen | (Compound 1:placebo) |

Substudy 1

| | | |
|---|---|---|
| Group 1 | 1.0 mg Compound 1 or placebo | 8 (6:2) |
| Group 2 | 3.0 mg Compound 1 or placebo | 8 (6:2) |
| Group 3 | 6.0 mg Compound 1 or placebo | 8 (6:2) |
| Group 4 | 12.0 mg Compound 1 or placebo | 8 (6:2) |
| Group 5 | 24.0 mg Compound 1 or placebo | 8 (6:2) |
| Group 7 | 36.0 mg Compound 1 or placebo | 8 (6:2) |
| Group 8 | 48.0 mg Compound 1 or placebo | 8 (6:2) |

Substudy 2

| | | |
|---|---|---|
| Group 6 | 3.0 mg Compound 1 | 12 |

PK parameters for single doses of Compound 1 in Substudy 1 are presented in Table 11. PK results for the ketoconazole and food effect evaluation in Substudy 2 are summarized below.

Specifically, Study M13-401 Substudy 2 evaluated the effect of ketoconazole, a strong CYP3A inhibitor, on the exposure of Compound 1 (drug-drug interaction study). The results showed that ketoconazole, a strong CYP3A inhibitor, increases Compound 1 AUC and Cₘₐₓ approximately 1.7- to 1.8-fold.

Study M13-401 Substudy 2 was also designed to evaluate the effect of food on the exposure of Compound 1. The results showed that food did not appear to affect the AUC of Compound 1−<1% decrease in AUC was observed in the presence of food.

Multiple-Dose Studies

Study M13-845 consisted of 3 substudies. In Substudy 1, healthy subjects received multiple oral dose administration of Compound 1 or placebo ranging from 3 mg to 24 mg twice daily for 14 days. In Substudy 2, subjects with mild to moderate RA on stable MTX treatment received multiple oral doses of Compound 1 or placebo for 4 weeks. In Substudy 3, healthy subjects received multiple doses of tofacitinib 5 mg twice daily for 14 days. Preliminary results are available from Substudy 1 (N=44).

TABLE 11
Pharmacokinetic Parameters (Mean ± SD) of Compound 1 Following Administration of Single Oral Doses of
Compound 1 to Healthy Subjects, Study M13-401 Substudy 1

| | Group 1 | Group 2 | Group 3 | Group 4 | Group 5 | Group 7 | Group 8 |
|---|---|---|---|---|---|---|---|
| Pharmacokinetic | 1 mg | 3 mg | 6 mg | 12 mg | 24 mg | 36 mg | 48 mg |
| Parameters (Units) | (N = 6) | (N = 6) | (N = 6) | (N = 6) | (N = 6) | (N = 6) | (N = 6) |
| Cₘₐₓ (ng/mL) | 7.72 ± 2.36 | 25.0 ± 6.88 | 38.9 ± 9.96 | 82.9 ± 12.1 | 158 ± 18.4 | 277 ± 44.5 | 314 ± 81.9 |
| Tₘₐₓ (h) | 1.3 ± 0.4 | 1.1 ± 0.2 | 1.1 ± 0.2 | 1.2 ± 0.4 | 1.3 ± 0.3 | 0.8 ± 0.3 | 0.8 ± 0.3 |
| t_{1/2} (h)^{a} | 2.6 ± 0.4^{b} | 5.9 ± 2.4 | 11.0 ± 3.4 | 12.1 ± 7.4 | 14.5 ± 9.0 | 6.4 ± 4.0 | 12.2 ± 3.52 |
| AUCₜ (ng · h/mL) | 29.8 ± 5.78 | 102 ± 27.5 | 159 ± 37.5 | 329 ± 48.9 | 612 ± 78.6 | 909 ± 201 | 1032 ± 174 |
| AUC_{∞} (ng · h/mL) | 30.1 ± 5.72 | 103 ± 27.6 | 160 ± 37.6 | 331 ± 49.8 | 615 ± 78.1 | 911 ± 202 | 1035 ± 174 |
| fₑ (%)^{c} | 17.7 ± 9.58 | 15.6 ± 4.44 | 15.6 ± 3.49 | 19.9 ± 2.26 | 18.6 ± 3.08 | 20.8 ± 9.25 | 16.4 ± 5.48 |

^{a}Terminal elimination half-life; harmonic mean (pseudo % CV).
^{b}Robust estimate of terminal elimination half-life may not have been achieved for this dose because Compound 1 concentrations fell below the lower limit of quantitation earlier than the higher dose levels.
^{c}fₑ % = percentage of Compound 1 dose recovered unchanged in urine.

The dose groups for Substudy 1 are shown in Table 12.

TABLE 12
Dose Groups in Substudy 1, Study M13-845

| | | | | Number of Subjects |
|---|---|---|---|---|
| Substudy | Group | Regimen | | (^{(Cpd. 1}:placebo) |
| Substudy 1 | Group 1 | 3 mg BID | Compound 1 | 11 (8:3) |
| | Group 2 | 6 mg BID | or placebo | 11 (8:3) |
| | Group 3 | 12 mg BID | | 11 (8:3) |
| | Group 4 | 24 mg BID | | 11 (8:3) |

BID = administered twice daily

Preliminary PK parameters for Compound 1 in Substudy 1 are presented in Table 13.

### Safety

Safety results from Study M13-401 are summarized below.

Safety Data from Phase I First-in-Human Study (Study M13-401)

Healthy adult male and female subjects (N=68) were enrolled in Study M13-401. Fifty-six subjects were enrolled and completed Substudy 1. Twelve subjects were enrolled and 11 subjects completed all three periods of Substudy 2.
Part 1 was a 2-period cross-over investigation designed to assess the effect of food on the safety and pharmacokinetics of a single oral dose of Compound 1 in healthy adult subjects.
Part 2 was a single period investigation designed to assess the potential metabolic interaction between ketoconazole and Compound 1.

One subject was prematurely discontinued from the study by the investigator due to nonclinically significant lab values for creatine phosphokinase after completion of Periods 1 and 2 in Part 1 of Substudy 2.

TABLE 13
Preliminary Mean (% CV) Pharmacokinetic Parameters of
Comp. 1 Following Multiple Oral Doses in Substudy 1,
Study M13-845

| | | Group 1 | | Group 2 | | Group 3 | | Group 4 | |
|---|---|---|---|---|---|---|---|---|---|
| | | 3 mg BID | | 6 mg BID | | 12 mg BID | | 24 mg BID | |
| Parameter | Units | N = 8 | | N = 8 | | N = 8 | | N = 8 | |
| | | Day 1 | | | | | | | |
| Tₘₐₓ | hr | 1.56 | (53) | 2.0 | (13) | 1.94 | (35) | 1.88 | (19) |
| Cₘₐₓ | ng/mL | 19.0 | (26) | 29.4 | (11) | 58.1 | (19) | 126 | (14) |
| AUC₁₂ | ng · hr/mL | 75.34 | (27) | 1.34 | (12) | 270 | (23) | 540 | (14) |
| Cₘₐₓ/dose | ng/mL/mg | 6.33 | (26) | 4.91 | (11) | 4.84 | (19) | 5.25 | (14) |
| C₁₂/dose | ng/mL/mg | 0.32 | (37) | 0.30 | (18) | 0.33 | (48) | 0.32 | (31) |
| AUC₁₂/dose | ng · hr/mL/mg | 25.1 | (27) | 22.3 | (12) | 22.5 | (23) | 22.5 | (14) |
| | | Day 14 | | | | | | | |
| Tₘₐₓ | hr | 1.69 | (55) | 2.13 | (21) | 2.19 | (24) | 1.75 | (15) |
| Cₘₐₓ | ng/mL | 18.5 | (29) | 28.8 | (13) | 57.6 | (19) | 119 | (14) |
| AUC₁₂ | ng · hr/mL | 78.3 | (26) | 138 | (12) | 271 | (19) | 529 | (12) |
| t_{1/2}^{a} | hr | 15.7 | (86) | 13.6 | (72) | 7.59 | (51) | 8.01 | (86) |
| Cₘₐₓ/dose | ng/mL/mg | 6.16 | (29) | 4.80 | (13) | 4.80 | (19) | 4.95 | (14) |
| AUC₁₂/dose | ng · hr/mL/mg | 26.1 | (26) | 23.1 | (12) | 22.6 | (19) | 22.0 | (12) |
| Fe % | | 18.8 | (27) | 18.7 | (31) | 21.4 | (18) | 18.7 | (32) |
| R_{ac} Cₘₐₓ^{b} | | 0.93 | (0.65-1.3) | 0.98 | (0.82-1.1) | 0.96 | (0.82-1.3) | 0.97 | (0.76-1.0) |
| R_{ac} AUC₀₋₁₂^{c} | | 1.1 | (0.87-1.2) | 1.0 | (0.87-1.2) | 1.0 | (0.88-1.1) | 1.0 | (0.78-1.3) |

BID = twice daily,
Fe % = percentage of unchanged drug recovered in urine
^{a}Harmonic mean ± pseudo % CV
^{b}R_{ac} Cₘₐₓ = Accumulation ratio (calculated as the ratio of Cₘₐₓ on Study Day 14 to Cₘₐₓ on Study Day 1); median and range (minimum to maximum) are presented.
^{c}R_{ac} AUC₀₋₁₂ = Accumulation ratio (calculated as the ratio of AUC₀₋₁₂ on Study Day 14 to AUC₀₋₁₂ on Study Day 1); median and range (minimum to maximum) are presented.

None of the adverse events in either Substudy 1 or Substudy 2 were severe, serious, or fatal, and none led to discontinuation of study drug. No clinically significant changes in laboratory values, vital signs, or ECG findings were observed during this study. No subjects in either Substudy 1 or 2 met the predefined criteria for potentially clinically significant ECG values for QT interval or QTcF interval. None of the ECG values for QT interval were considered clinically significant or abnormal by the investigator or the medical monitor and were not associated with adverse events or discontinuation from the study.

Preliminary Data from Phase I Study in Healthy Volunteers and Patients with Rheumatoid Arthritis (Study M13-845)

Composed of 3 substudies, enrollment for Substudy 1 and Substudy 3 (both performed in healthy volunteers) is completed, and enrollment for Substudy 2 (performed in subjects with RA receiving a stable dose of MTX) was terminated prematurely due to enrollment challenge. However, 14 subjects have completed Substudy 2. None of adverse events (AEs) reported in these 2 substudies were severe, serious, or fatal, and none led to discontinuation of study drug. In addition, no clinically significant changes in laboratory values, vital signs, or electrocardiogram (ECG) findings were observed.

Substudy 1 evaluated the administration of placebo or Compound 1 doses of 3, 6, 12, and 24 mg twice daily for 14 days in healthy volunteers. 6 out of 42 subjects (14.3%) who received Compound 1 and 3 out of 14 subjects (16.7%) who received placebo reported treatment emergent AEs (TEAEs). AEs reported by more than 1 subject who received Compound 1 were headache (2 subjects in the Compound 1 48 mg dose group) and presyncope (1 subject in each of the Compound 1 6 mg and 24 mg dose groups). All other AEs in the combined Compound 1 dose groups were reported by 1 subject: diarrhea, application site dermatitis, dizziness, and epistaxis. All events were assessed as mild in severity; only headache and diarrhea in the Compound 1 groups were considered as having a reasonable possibility of being study drug related.

In Substudy 3, which evaluated the administration of tofacitinib 5 mg twice daily for 14 days in healthy volunteers, 1 adverse event (pruritus) was reported (occurring in 1 subject).

Substudy 2 was designed as a randomized, double-blind, parallel-group, placebo controlled study to assess the safety, tolerability, and PK of multiple doses of Compound 1 in subjects with mild to moderate RA who are on stable MTX treatment. The study was designed to enroll approximately 32 subjects randomized in a 1:1:1:1 fashion to 4 groups (placebo, 6 mg BID, 12 mg BID, and 24 mg BID), but was terminated prematurely due to enrollment challenge. Among the 14 subjects who enrolled and completed the study (placebo [n=4]; Compound 1 6 mg BID [n=4], Compound 1 12 mg BID [n=3], Compound 1 24 mg BID [n=3]), 9 AEs in 7 subjects were reported. Two events (fatigue and vomiting) occurred in the placebo group and 7 events (nausea, vomiting, upper respiratory tract infection, gastroenteritis, post-traumatic neck syndrome, back pain, and insomnia) occurred in the combined Compound 1 treatment groups, all of which were reported as not related to Compound 1 treatment. All AEs were considered either mild or moderate in severity.

In summary, safety data for healthy volunteers administered Compound 1 in single doses ranging from 1 to 48 mg and multiple doses of 3, 6, 12, and 24 mg twice daily for 14 days revealed no dose-limiting toxicities. Compound 1 was well tolerated at all doses. None of the adverse events reported were severe, serious, or fatal, and none led to discontinuation of study drug. In addition, no clinically significant changes in laboratory values, vital sign measurements, or ECG findings were observed.

Compound 1 is a novel Jak1 selective inhibitor. Inhibition of Jak1 blocks the signaling of many pro-inflammatory cytokines including IL-6, IL-2, IL-7, and IL-15. In a rat AIA model, Compound 1 halted disease progression, an effect that correlated with decreases in the numbers of NK cells as well as CD8⁺ and CD25⁺ T cells. These preclinical experiments, as well as published data with other Jak inhibitors, indicate that Compound 1 offers promise as a treatment for patients with RA. The selectivity of Compound 1 for Jak1 over Jak2 is hypothesized to improve upon the therapeutic profile of agents that are less discriminatory.

The available data suggests that the greater selectivity of Compound 1 in preclinical studies may lead to an improved clinical profile in patients with RA and other inflammatory or autoimmune disorders, at doses that produce less hematologically-related adverse events compared with other Jak inhibitors.

In vitro studies indicated that the metabolism of Compound 1 is expected to be mediated by CYP 3A4, 3A5, and 2D6. In order to elucidate the influence of strong CYP3A4/5 inhibitors on Compound 1 exposure, a drug-drug interaction study was completed with ketoconazole. Preliminary clinical study results suggest that coadministration of Compound 1 with ketoconazole, a strong CYP3A inhibitor, increased Compound 1 Cₘₐₓ and AUC approximately 1.7- to 1.8-fold. Therefore, coadministration of strong CYP3A inhibitors with Compound 1 is expected to result in a moderate increase in Compound 1 exposure.

In vitro, Compound 1 is not an inhibitor of CYP 1A2, 2B6, 2C8, 2C9, 2C19, 2D6, or 3A4 at concentrations up to 30 μM. Thus, clinically relevant changes in exposures to concomitant medications, which are substrates of these pathways, are not anticipated.

Compound 1 was tested in a battery of safety pharmacology assays. In dogs, Compound 1 produced moderate dose-dependent effects on mean arterial pressure and heart rate, beginning at a Cₘₐₓ of 0.42 μg/mL. However, no effects on blood pressure and heart rate have been observed in the FIH study through the highest single dose of 48 mg (Cₘₐₓ=0.31 μg/mL).

The toxicity profile of Compound 1 has been evaluated in repeated-dose studies in rats and dogs, including 4-week studies and chronic studies in both species (6 months in rats and 9 months in dogs). In the pivotal 4-week studies, Compound 1-related effects included decreases in circulating lymphocytes and decreased cellularity of lymphoid tissues as well as suppression of erythropoiesis with resultant decreases in reticulocytes and red cell mass. In nonclinical toxicity studies, the dog has been identified as the most sensitive species.

In the 9-month toxicity study in dogs, no adverse effects of Compound 1 administration were identified at daily dosages up to 1.5 mg/kg/day. The only direct effect of Compound 1 administration was a minimal to mild, non-adverse decrease in red cell mass at 0.5 and 1.5 mg/kg/day from Dosing Day 28 to 182. At the end of dosing (Day 265), decreases in red cell mass were either similar to or less than decreases reported on Day 91, suggesting these decreases either improved or did not worsen with continued dosing. The inflammatory findings in the paws of dogs with subsequent inflammation in draining lymph nodes are not considered relevant for humans since they are associated with housing dogs on cage flooring. Based on the lack of any adverse findings in the 9-month study, the NOAEL was 1.5 mg/kg/day, which resulted in an average (male and female) Day 272 Cₘₐₓ of 212 ng/mL and AUC of 888 ng·hr/mL.

In a 6-month toxicity study in rats. Mild decreases in red cell mass and reticulocytes were observed in both sexes, mainly at 50 mg/kg/day. These mild decreases in red cell mass were considered non-adverse based on magnitude. NOAEL following 6 months of dosing in rats was determined to be 20 mg/kg/day in males and females, resulting in Cₘₐₓ values of 1.11 and 2.24 μg/mL, respectively, and AUC₀₋₂₄ values of 3.83 and 6.84 μg·hr/mL, respectively.

Published studies have shown that Jak1 pathway inhibition is associated with effects on interleukin signaling, whereas Jak2 inhibition is associated with impairment of erythroid development and other pathways including thrombopoietin, which regulates platelet production, and angiotensin, which plays an important role in regulating vascular tone. In humans, Jak3 deficiency is associated with an autosomal recessive form of severe combined immunodeficiency (SCID) and is characterized by a lack of circulating T cells and natural killer cells, but a normal number of B cells. Patients with SCID due to Jak3 deficiency typically present as infants with failure to thrive, severe or recurrent diarrhea, and respiratory infections.

On the basis of the information above as well as the results from other animal studies, Compound 1 administration is expected to be associated with effects on WBCs and cytokine signaling in humans. These immunomodulatory effects may result in an increased risk of infection. Studies of other less selective Jak inhibitors in RA patients have reported increases in serum creatinine, in total, LDL, and HDL cholesterol, and in liver transaminases. The increases in serum creatinine, lipids, and liver transaminase values typically have been asymptomatic, reversible, and not associated with any overt declines in renal or hepatic function (Riese et al., Best Pract. Res. Clin. Rheumatol. 24(4):513-526, 2010; Fleischmann et al., Arthritis Rheum. 63:LB3, 2011). Also, events of gastrointestinal perforation have been reported in clinical studies with the Jak inhibitor tofacitinib in RA patients. Per the prescribing information, tofacitinib should be used with caution in patients who may be at increased risk for gastrointestinal perforation (e.g., patients with a history of diverticulitis).

In addition, drugs that affect the immune response may increase the risk of malignancy, especially with longer term dosing. In the tofacitinib (a nonselective inhibitor of Jak1, Jak2, and Jak3) clinical studies, lymphoma, and other malignancies have been reported.

Results are available regarding the PK and safety of Compound 1 in humans from Study M13-401, which was composed of 2 substudies. Substudy 1 evaluated 7 single dose levels of Compound 1 ranging from 1 mg to 48 mg. Compound 1 was rapidly absorbed, with a median Tₘₐₓ of approximately 1 hour in the fasted state. After reaching Cₘₐₓ, Compound 1 appeared to decline in a biphasic fashion. Compound 1 concentrations rapidly decrease following Tₘₐₓ followed by an elimination phase with a t½ of approximately 3 to 15 hours. In Substudy 2 (food effect/ketoconazole interaction), 12 subjects received 3 mg of Compound 1 under fasting and nonfasting conditions and concomitantly with ketoconazole. Food appeared to have no effect on Compound 1 (AUC); <1% decrease in AUC in the presence of food. Therefore, Compound 1 can be administered with and without food. Preliminary clinical study results suggest that coadministration of Compound 1 with ketoconazole, a strong CYP3A inhibitor, increased Compound 1 Cₘₐₓ and AUC approximately 1.7- to 1.8-fold. Therefore, coadministration of strong CYP3A inhibitors with Compound 1 is expected to result in a moderate increase in Compound 1 exposure. The study protocol should be consulted for guidance prior to coadministration of Compound 1 with known strong CYP3A inhibitors or strong CYP3A inducers.

Preliminary results from multiple dose Study M13-845 suggest that Compound 1 had minimal to no accumulation, with a median Tₘₐₓ of approximately 2 hours followed by an elimination phase with a t½ of approximately 8 to 16 hours following the last dose on Day 14. Compound 1 exposure appeared to be approximately dose-proportional.

Approximately 19% to 21% of Compound 1 was excreted as unchanged parent drug in the urine during a dose interval at steady state. Preliminary safety data for healthy volunteers administered Compound 1 in single doses ranging from 1 to 48 mg (Study M13-401) and multiple doses of 3, 6, 12, and 24 mg twice daily for 14 days (Study M13-845, Substudy 1) revealed no dose-limiting toxicities.

Compound 1 was well-tolerated at all doses. None of the adverse events reported were severe, serious, or fatal and none led to discontinuation of study drug. In addition, no clinically significant changes in laboratory values, vital sign measurements, or ECG findings were observed. None of the ECG values for QT interval or QTcF interval were considered clinically significant or abnormal by the investigator or the medical monitor and were not associated with adverse events or discontinuation from the study.

In the Compound 1 clinical trials, safety monitoring of all subjects includes physical examinations, blood chemistry and hematology assessments, urinalyses, electrocardiograms, and vital sign measurements including heart rate and blood pressure. Adverse event evaluation will be performed throughout the studies. Laboratory evaluations will include complete blood cell counts including platelet counts, reticulocyte counts, hepatic panels, serum creatinine, lipid profiles, and lymphocyte subsets. Subjects with clinically significant hematological abnormalities, hepatic or renal dysfunction and significant cardiovascular disease (as specified in the protocol) will be excluded from the initial clinical studies.

Because of the potential increased risk of infection, subjects with an active or chronic infection or recent receipt of a live vaccine will be excluded from the studies. Also, subjects should not receive a live viral vaccine while participating in the Compound 1 studies. Subjects should be closely monitored for infection during and after treatment with study drug and, if signs and symptoms develop, undergo prompt diagnostic testing appropriate for an immunocompromised subject. Study drug should be interrupted if a subject develops a serious infection or an opportunistic infection. As appropriate, antimicrobial therapy should be initiated, and the subject closely monitored.

Viral reactivation, including cases of herpes virus reactivation (e.g., herpes zoster), have been observed in clinical studies with other Jak inhibitors. In the initial Compound 1 studies, subjects with evidence of chronic hepatitis B or C infection should be excluded. In addition, all subjects should be evaluated for tuberculosis (as per protocol) before receiving study drug in the Compound 1 studies; subjects with evidence of active or untreated latent tuberculosis should be excluded from the studies.

In preclinical studies, Compound 1 has shown photoinstability and high molar absorptivity values; tissue distribution studies indicate that Compound 1 is well distributed to skin. In repeated-dose oral toxicity studies in rats and dogs of 6 and 9 months duration, respectively, no evidence of skin or ocular toxicity has been observed. Furthermore, Compound 1 was negative for phototoxicity potential in a neutral red uptake phototoxicity assay in Balb/c 3T3 mouse fibroblasts.

Monitoring of subjects will occur after the end of dosing (for a minimum of 30 days) and subject hematology or chemistry values outside the reference range at the last follow-up visit, which the investigator considers to be a clinically significant change, should be followed to a satisfactory clinical resolution. Toxicity management guidelines for selected laboratory values (hemoglobin, absolute lymphocyte count, absolute neutrophil count, total WBC count, platelet count, alanine aminotransferase/aspartate aminotransferase, serum creatinine) are provided in Phase 2 protocols.

### Example 11

### Treatment of Moderately to Severely Active Crohn's Disease in Patients Who have Inadequately Responded to or are Intolerant to Anti-TNFα Therapy

The following example briefly describes treatment of subjects with moderately to severely active Crohn's disease (CD) who have inadequately responded to or are intolerant to an anti-TNFα therapy.

Male and female adult patients with a diagnosis of moderately to severely active Crohn's disease, with evidence of mucosal inflammation, defines as (1) having a Simplified Endoscopic Score for Crohn's disease (SES-CD)≧6 (or SES-CD≧4 for patients with disease limited to the ileum), and (2) average daily liquid/soft stool frequency ≧2.5 or average daily abdominal pain score ≧2.0, and 220≦CDAI≦450, are included in the study. The adult patients may also have a history of inadequate response to or are intolerance to anti-TNF therapy. Up to 35% of subjects may be primary non-responders to anti-TNFα treatment.

Criteria for inadequate response to previous treatment or intolerance to previous treatment with an anti-TNFα agent includes:
Signs and symptoms of persistently active disease despite a history of at least one 4-week induction regimen of one of the following agents:
   Infliximab: 5 mg/kg IV, 2 doses at least 2 weeks apart;
   Adalimumab: one 160 mg s.c. dose followed by one 80 mg s.c. dose (or one 80 mg s.c. dose) followed by one 40 mg dose at least 2 weeks apart;
   Certolizumab pegol: 400 mg s.c., 2 doses at least 2 weeks apart; or,
Recurrence of symptoms during scheduled maintenance dosing following prior clinical benefit (discontinuation despite clinical benefit does not qualify); or,
History of intolerance of at least one TNFα antagonist (including, but not limited to infusion-related reaction, demyelination, congestive heart failure, infection)

Enrolled subjects receive one of the test doses of Compound 1, such as 3, 6, 9, 12, 18, or 24 mg BID or QD or placebo.

Criteria for inadequate response are as follows:
Average daily liquid/soft stool frequency >2.2 OR average daily abdominal pain score >1.8 AND
An increase level of hs-CRP of at least 1 mg/L from baseline or a hs-CRP≧5 mg/L.

### Example 12

### Preliminary Pharmacokinetic (PK) Results for Compound 1 in Healthy Japanese and Chinese Subjects (Study M13-543)

The PK of Compound 1 was assessed after single escalating oral doses of 1.0 mg, 6.0 mg, and 24.0 mg were administered to three groups of volunteer Japanese subjects, with 6 subjects in each dose level. Two additional subjects were assigned to each dose level as placebo control.

In addition, healthy Japanese or Chinese subjects received multiple oral dose administration of 18 mg/dose of Compound 1 or placebo twice daily for 14 days (BID). In one group, 8 Japanese subjects received 18 mg/dose over the 2-week period, and 2 additional subjects were included as placebo control. In another group, 8 Chinese subjects received 18 mg/dose over the 2-week period, and 2 additional subjects were included as placebo control.

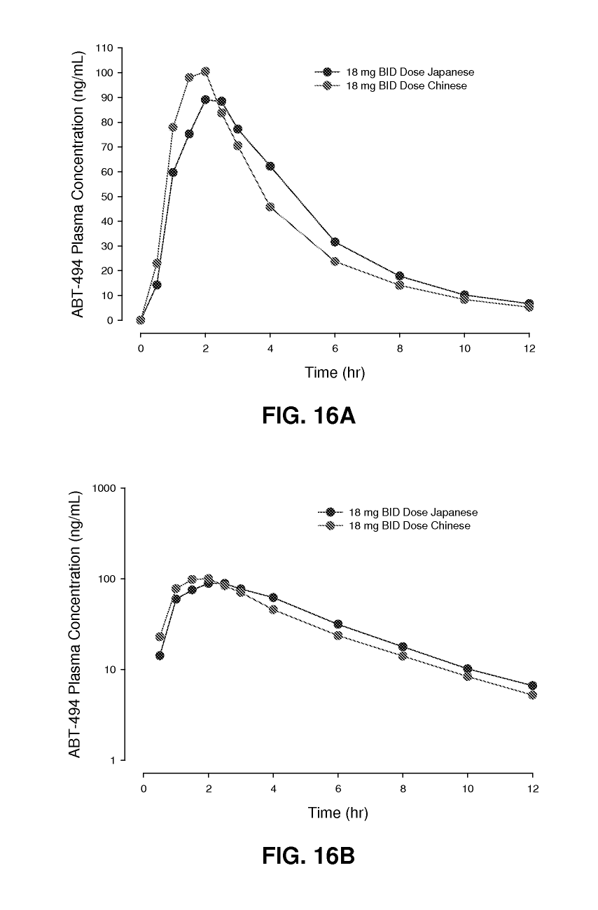

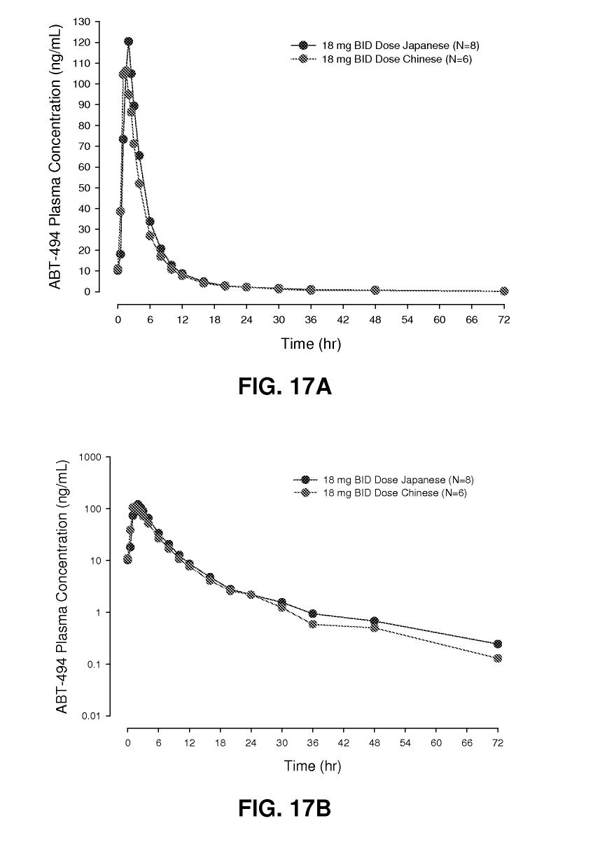

The PK values of Compound 1 for all the groups above were assessed after the period ended. Preliminary mean (% CV) Compound 1 pharmacokinetic parameters are presented below in Table 14. In addition, preliminary Compound 1 mean PK profile in healthy Japanese and Chinese subjects at Day 1 (AM) and Day 14 (AM), after taking 18 mg Compound 1 BID, shows that the plasma concentration of Compound 1 over 12 hrs (Day 1) or 72 hrs (Day 14) in both ethnic groups are shown in FIGS. 16A-17B.

A comparison of Compound 1 dose-normalized exposure and terminal half-life was also made in healthy Japanese, Chinese and Western subjects following multiple oral BID dosing. The results are summarized below in Table 15.

Data presented herein suggests that:
Following multiple dose administration, Compound 1 dose-normalized exposures appeared to be comparable in Japanese, Chinese, and Western healthy subjects, although:
   Compound 1 dose-normalized AUC and C_{trough} were approximately 20% higher in Asians than Westerns
   Compound 1 dose-normalized Cₘₐₓ were approximately 40% higher in Asians than Westerns
Similar to previous observations in Western subjects, Compound 1 displayed minimal accumulation with multiple BID dosing in Japanese and Chinese healthy subjects
Compound 1 terminal t_{1/2} was comparable in Japanese, Chinese, and Western healthy subjects (approximately 7 to 9.5 hours)

Similarly, preliminary mean (% CV) Compound 1 PK parameters following single dose administration in healthy Japanese subjects compared to Western subjects are summarized below in Table 16.

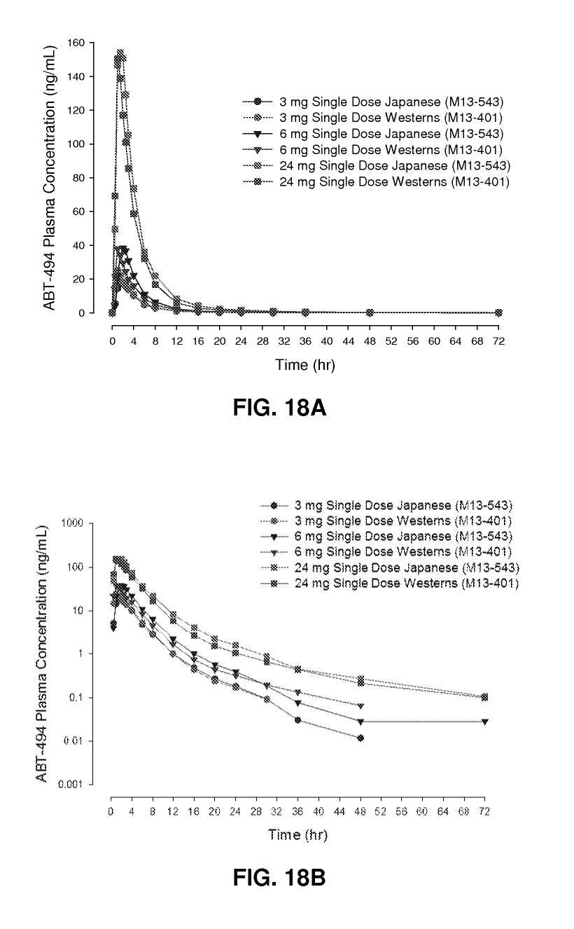

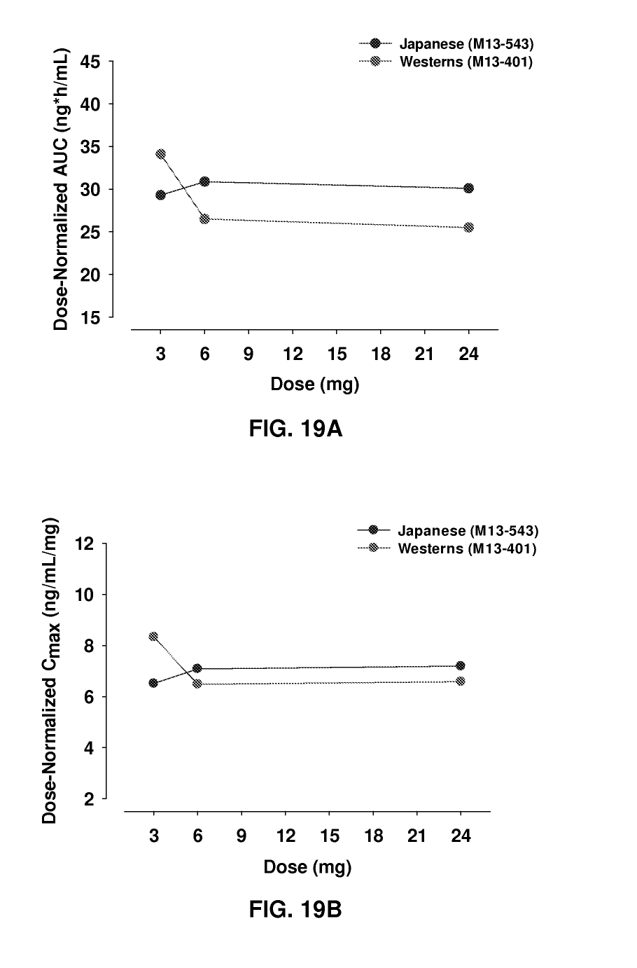

Preliminary Compound 1 PK profile (mean) in healthy Japanese subjects compared to Western subjects is shown in FIGS. 18A-B. Preliminary Compound 1 dose normalized AUC and Cₘₐₓ in healthy Japanese subjects compared to Western subjects are shown in FIGS. 19A-B.

The data obtained from the study above suggests that:
Compound 1 exposures following 3, 6, and 24 mg single doses in Japanese subjects were comparable to previously observed exposures in Western subjects
   At the 3 mg SD, Compound 1 Cₘₐₓ and AUC were 22% and 14% lower, respectively, than previously observed in Western subjects
   At the 6 & 24 mg single doses, Compound 1 Cₘₐₓ and AUC were 9% and 16-18% higher, respectively, than previously observed in Western subjects
   These difference are within the range of study-to-study variability
Compound 1 Cₘₐₓ and AUC increased in Japanese subjects in an approximately dose-proportional manner between 3 and 24 mg single doses.

TABLE 14

| | | Day 1 | | Day 14 | | | |
|---|---|---|---|---|---|---|---|
| | | Group 4 | Group 5 | Group 4 | | Group 5 | |
| | | 18 mg BID | 18 mg BID | 18 mg BID | | 18 mg BID | |
| | | Japanese Subjects | Chinese Subjects | Japanese Subjects | | Chinese Subjects | |
| PK Parameter | Units | N = 8 | N = 7 | N = 8 | | N = 6 | |
| Cₘₐₓ | ng/mL | 107 (36) | 116 (39) | 128 | (18) | 118 | (23) |
| Tₘₐₓ | hr | 2.1 (27) | 1.7 (29) | 2.0 | (24) | 1.6 | (37) |
| AUC₁₂ | ng · hr/mL | 440 (18) | 410 (21) | 522 | (15) | 466 | (10) |
| t_{1/2}^{a} | hr | — | — | 9.51 | (70) | 6.88 | (54) |
| C_{trough} | ng/mL | — | — | 8.61 | (33) | 7.70 | (22) |
| CL/F | L/hr | — | — | 35.2 | (15) | 39.0 | (10) |
| Cₘₐₓ/Dose | ng/mL/mg | 5.97 (36) | 6.45 (39) | 7.08 | (18) | 6.57 | (23) |
| AUC₁₂/Dose | ng · hr/mL/mg | 24.5 (18) | 22.8 (21) | 29.0 | (15) | 25.9 | (10) |
| C_{trough}/Dose | ng/mL/mg | — | — | 0.48 | (33) | 0.43 | (22) |
| R_{ac} Cₘₐₓ^{b} | — | — | — | 1.2 | (0.6-2.0) | 1.1 | (0.7-1.3) |
| R_{ac} AUC₀₋₁₂^{c} | — | — | — | 1.2 | (1.0-1.5) | 1.2 | (1.0-1.3) |

^{a}Harmonic mean and pseudo % CV
^{b}R_{ac} Cₘₐₓ = Accumulation ratio (calculated as the ratio of Cₘₐₓ on Study Day 14 to Cₘₐₓ on Study Day 1); median and range
^{c}R_{ac} AUC₀₋₁₂ = Accumulation ratio (calculated as the ratio of AUC₀₋₁₂ on Study Day 14 to AUC₀₋₁₂ on Study Day 1); median and range

TABLE 15

| | | | | | |
|---|---|---|---|---|---|
| | | 18 mg BID | 18 mg BID | 12 mg BID | 24 mg BID |
| | | Japanese Subjects | Chinese Subjects | Western Subjects^{a} | Western Subjects^{a} |
| PK Parameter | Units | N = 8 | N = 6 | N = 8 | N = 8 |

Day 14

| | | | | | |
|---|---|---|---|---|---|
| Cₘₐₓ/Dose | ng/mL/mg | 7.08 (18) | 6.57 (23) | 4.80 (19) | 4.95 (14) |
| AUC₁₂/Dose | ng · hr/mL/mg | 29.0 (15) | 25.9 (10) | 24.2 (20) | 24.1 (13) |
| C_{trough}/Dose | ng/mL/mg | 0.48 (33) | 0.43 (22) | 0.38 (34) | 0.40 (28) |
| t_{1/2}^{b} | hr | 9.51 (70) | 6.88 (54) | 7.6 (63) | 8.0 (53) |

^{a}18 mg BID regimen was not evaluated in Western healthy subjects; therefore, dose-normalized exposures for 12 and 24 mg in Western subjects are used for the comparison.
^{b}Harmonic mean and pseudo % CV

TABLE 16

| | | Group 1 | | Group 2 | M13-401 | Group 3 | M13-401 |
|---|---|---|---|---|---|---|---|
| | | (3 mg) | M13-401 (3 mg) | (6 mg) | (6 mg) | (24 mg) | (24 mg) |
| | | Japanese | Western | Japanese | Western | Japanese | Western |
| PK | | Subjects | Subjects^{d} | Subjects | Subjects^{d} | Subjects | Subjects^{d} |
| Parameter | Units | (N = 6) | (N = 6) | (N = 6) | (N = 6) | (N = 6) | (N = 6) |
| Cₘₐₓ | ng/mL | 19.5 (27) | 25.0 (28) | 42.5 (13) | 38.99 (26) | 173 (21) | 158 (12) |
| Tₘₐₓ | hr | 1.9 (42) | 1.1 (19) | 2.0 (22) | 1.1 (19) | 1.5 (30) | 1.3 (22) |
| AUCₜ | ng · hr/mL | 87.9 (17) | 102 (27) | 185 (12) | 159 (24) | 722 (32) | 612 (13) |
| t_{1/2}^{a} | hr | 6.1 (27)^{b} | 5.9 (40)^{b} | 5.5 (40)^{b,c} | 11.0 (31)^{c} | 14.0 (93) | 14.5 (62) |
| Cₘₐₓ/Dose | ng/mL/mg | 6.51 (27) | 8.33 (28) | 7.1 (13) | 6.48 (26) | 7.19 (21) | 6.58 (62) |
| AUCₜ/Dose | ng · hr/mL/mg | 29.3 (17) | 34.1 (27) | 30.9 (12) | 26.5 (24) | 30.1 (32) | 25.5 (13) |

^{a}Harmonic mean and pseudo % CV
^{b}Not a robust estimate for terminal elimination t_{1/2}. Concentrations at the terminal phase were below LLOQ in most subjects.
^{c}The apparent difference in the half-life for the 6 mg dose between Western and Japanese is due to difference in the interval over which the elimination phase is characterized
^{d}Compound 1 PK parameters following single dose administration in Western subjects were determined in the SAD study M13-401. Single doses of Compound 1 were administered to Western subjects under fasting conditions.

The teachings of all references, including journal articles, patents and published patent applications, are incorporated herein by reference in their entirety.

1. A method of selectively inhibiting a Janus Kinase 1 (Jak1) in a human, comprising administering to said human an effective amount of the free base form of a compound,
wherein Jak1 activity is preferentially inhibited over activity of Jak2, activity of Jak3, and activity of Tyk2, and less than 50%, 40%, 30%, 20%, 10%, or 5% of Jak2 and/or Jak3 activity is inhibited in the human, and
wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

2. The method of claim 1, wherein more than 50%, 60%, 70%, 80%, 90%, 95%, 99% of Jak1 activity is inhibited in said human.

3-4. (canceled)

5. The method of claim 1, wherein the human is in need of treatment for a condition treatable by inhibition of Jak1 activity, and wherein the condition is Rheumatoid Arthritis (RA), Crohn's disease, ankylosing spondylitis (AS), psoriatic arthritis, psoriasis, ulcerative colitis, systemic lupus erythematosus (SLE), lupus nephritis, diabetic nephropathy, dry eye syndrome, Sjogren's Syndrome, alopecia areata, vitiligo, or atopic dermatitis.

6. The method of claim 5, wherein the Crohn's disease is moderately to severely active Crohn's disease (CD) in an adult.

7. The method of claim 6, wherein the adult is newly diagnosed of CD, or is inadequately responding to or has discontinued therapy due to loss of response to or intolerance to a first line therapy or an anti-TNFα therapy.

8. The method of claim 7, wherein the adult is inadequately responding to or has discontinued therapy due to loss of response to or intolerance to: azathioprine, 6-mercaptopurine (6-MP), aminosalicylate, sulfasalazine, mesalamine, corticosteroid, prednisone, prednisone equivalent, budesonide, probiotic, methotrexate, cyclosporine, tacrolimus, metronidazole, ciprofloxacin, leflunomide, chloroquine, hydroxychloroquine, penicillamine, tocilzumab, anakinra, abatacept, rituximab, efalizumab, belimumab, tofacitinib, baricitinib, golimumab, vedolizumab, natalizumab, ustekinumab, etanercept, infliximab, adalimumab, certolizumab pegol, or a JAK inhibitor.

9. The method of claim 5, wherein the RA is moderately to severely active RA in an adult.

10. The method of claim 9, wherein RA-associated bone erosion in the adult is inhibited.

11. The method of claim 9, wherein the adult is newly diagnosed of RA, is inadequately responding to oral or biologic DMARDs, or has discontinued therapy due to loss of response to or unacceptable toxicity from methotrexate, chloroquine, azathioprine, hydroxychloroquine, penicillamine, sulfasalazine, leflunomide, tocilzumab, anakinra, abatacept, certolizumab pegol, tofacitinib, golimumab, baricitinib, etanercept, infliximab, or adalimumab.

12. The method of claim 1, wherein the method does not substantially reduce NK cell count, NKT cell count, and/or iNKT cell count.

13. The method of claim 1, wherein the method does not substantially inhibit erythropoiesis, granulocyte/monocyte-colony stimulating factor (GM-CSF) signaling, or emergency myelopoiesis in response to microbial infection in said human.

14. The method of claim 1, wherein the human has anemia, or a whole blood hemoglobin level of less then 12, 11, 10, 9, 8, 7, 6, or 5 g/dL.

15. The method of claim 1, wherein the compound is administered to said human until a substantially steady level of AUC₀₋₂₄ of between 0.10-1.1 μg·hr/mL of free base equivalent of the compound is reached.

16. The method of claim 15, wherein the compound is administered to said human twice daily (BID) in equal amounts.

17. The method of claim 16, wherein the compound is administered to said human twice daily, each time at a dose of about 3-24 mg of free base equivalent of the compound.

18. The method of claim 15, further comprising maintaining the AUC₀₋₂₄ at substantially the same level over a treatment period.

19. (canceled)

20. The method of claim 1, wherein inhibition of Jak1 activity is determined by measuring ex vivo stimulated IL-6 dependent STAT3 phosphorylation, ex vivo stimulated IL-7-dependent STAT5 phosphorylation, and/or by determining peripheral NK cell counts.

21. The method of claim 1, wherein inhibition of Jak2 activity is determined by measuring GM-CSF dependent STAT5 phosphorylation.

22. The method of claim 1, further comprising administering to the human one or more additional agents which modulate a mammalian immune system or which are anti-inflammatory agents.

23. The method of claim 22, wherein said one or more additional agents is selected from the group consisting of: aspirin, acetaminophen, aminosalicylate, ciprofloxacin, corticosteroid, cyclosporine, metronidazole, probiotic, tacrolimus, ibuprofen, naproxen, piroxicam, prednisolone, dexamethasone, anti-inflammatory steroid, methotrexate, chloroquine, azathioprine, hydroxychloroquine, penicillamine, sulfasalazine, leflunomide, tocilzumab, anakinra, abatacept, certolizumab pegol, golimumab, vedolizumab, natalizumab, ustekinumab, rituximab, efalizumab, belimumab, etanercept, infliximab, adalimumab, or an immune modulator for CD4⁺CD25⁺ T_{reg} cells.

24. A method of treating in a human an autoimmune disease or disorder, or an inflammatory disease or disorder, the method comprising administering to said human an effective amount of the free base form of a compound,
wherein the effective amount reduces reticulocyte, NK cell, NKT cell, iNKT cell, or CD8⁺ cell count by no more than 50%, 40%, 30%, 20%, 10%, 5% relative to a pre-treatment level, and,
wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

25. A method of treating in a human an autoimmune disease or disorder, or an inflammatory disease or disorder, the method comprising administering to said human an effective amount of the free base form of a compound,
wherein the effective amount produces an AUC₀₋₂₄ of between 0.10-1.1 μg·hr/mL (or between 0.128-1.058 μg·hr/mL) of free base equivalent of the compound, and,
wherein the compound is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide.

26-44. (canceled)

45. The method of claim 1, further comprising:
(1) identifying a human subject administered with the compound but having inadequate or suboptimal response or therapeutic efficacy;
(2) determining reticulocyte, NK cell, NKT cell, iNKT cell, and/or CD8⁺ cell count of the human subject, wherein a decrease in reticulocyte, NK cell, NKT cell, iNKT cell, or CD8⁺ cell count of no more than 30%, 25%, 20%, 15%, or 10% compared to a pre-treatment baseline level of reticulocyte, NK cell, NKT cell, iNKT cell, or CD8⁺ cell count, respectively, is indicative that the human subject is a candidate for dose escalation;
(3) administering to said candidate an escalated dose of the compound.

46. The method of claim 45, further comprising repeating steps (1)-(3) until a desired outcome is achieved.

47. A pharmaceutical formulation for treating an autoimmune disease or disorder, or an inflammatory disease or disorder, the pharmaceutical composition comprising: (1) a unit dose of the free base form of the compound (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, and (2) a pharmaceutically acceptable excipient, wherein the unit dose, upon administration to an adult human twice daily (BID), produces an AUC₀₋₂₄ of between 0.10-1.1 μg·hr/mL of free base equivalent of the compound.

48. (canceled)

49. The pharmaceutical formulation of claim 47, wherein the unit dose is 0.5, 1, 3, 6, 9, 12, 18, or 24 mg of free base equivalent of the compound.

50. The pharmaceutical formulation of claim 47, wherein the autoimmune disease or disorder, or inflammatory disease or disorder is Crohn's disease in adult.

51. The pharmaceutical formulation of claim 47, wherein the autoimmune disease or disorder, or inflammatory disease or disorder is Rheumatoid Arthritis (RA) in adult.

52. (canceled)

53. The pharmaceutical formulation of claim 47, which is formulated for oral, topical, dermal, intra-luminal, or ophthalmic administration.


SEQUENCE LISTING (2 sequences)

SEQ ID NO: 1 - protein, 124 aa, Mus musculus
EEQLVESGGGLVKPGGSLRLSCAASGFSFSDCRMYWLRQAPGKGLEWIGVISVKSENYGANYAESVRGRFTISRDDSKNTVYLQMNSLKTEDTAVYYCSASYYRYDVGAWFAYWGQGTLVTVSS

SEQ ID NO: 2 - protein, 111 aa, Mus musculus
DIVMTQSPDSLAVSLGERATFNCRASKSVSTSGYSYIYWYQQKPGQPPKLLIYLASILESGVPDRFSGSGSGTDFTLTISSLQAEDVAVYYCQHSRELPWTFGQGTKVEIK